(19) United States
(12) Patent Application Publication
(10) Pub. No.: US 2026/0216371 A1
(43) Pub. Date: Jul. 30, 2026

(54) NUCLEIC ACID DELIVERY COMPOSITIONS

(71) Applicant: EARLI Inc., Redwood City, CA (US)

(72) Inventors: Badriprasad ANANTHANARAYANAN, Albany, CA (US); Blaine MCCARTHY, Burlingame, CA (US); Sushil LATHWAL, Mountain View, CA (US); Shengshuang ZHU, Lexington, MA (US)

(21) Appl. No.: 19/646,336

(22) Filed: Apr. 13, 2026

Related U.S. Application Data
(63) Continuation of application No. 19/551,454, filed on Feb. 26, 2026.
Application No. 19/551,454 is a continuation of application No. PCT/US2024/044226, filed on Aug. 28, 2024.
(60) Provisional application No. 63/579,480, filed on Aug. 29, 2023.

(51) Int. Cl. A61K 48/00

(52) U.S. Cl. CPC A61K 48/0041; A61K 48/005

(57) ABSTRACT

Compositions and methods for delivering nucleic acids (e.g., DNA) to cells are described herein. In some embodiments, such compositions and methods involve the use of lipid nanoparticles (LNPs).

### CROSS-REFERENCE

This application is a Continuation of U.S. application Ser. No. 19/551,454, filed Feb. 26, 2026, which is a Continuation of International Application No. PCT/US2024/044226, filed Aug. 28, 2024, which claims the benefit of U.S. Provisional Application No. 63/579,480, entitled “NUCLEIC ACID DELIVERY COMPOSITIONS” and filed on Aug. 29, 2023, each of which is incorporated by reference herein in its entirety.

### INCORPORATION BY REFERENCE OF SEQUENCE LISTING

The present application is being filed along with a Sequence Listing in electronic format. The Sequence Listing is provided as a file entitled 53531-716_302_SL.xml, created Mar. 19, 2026, which is 2,908 bytes in size. The information in the electronic format of the Sequence Listing is incorporated by reference in its entirety.

### BACKGROUND

The delivery of nucleic acids intended to bring about a desired reaction in vivo presents numerous challenges. Lipid nanoparticles (LNPs) containing one or more types of lipids (e.g., cationic lipids, helper lipids, and PEG-modified lipids) are being used to improve nucleic acid delivery in vivo.

### SUMMARY

Nucleic acid-based therapeutics have enormous potential, but there remains a need to deliver nucleic acids more effectively to the appropriate sites within an organism to realize this potential. Accordingly, described herein are methods, compositions, and systems for improved packaging and/or delivery of nucleic acids (e.g., DNA).

In some aspects, the present disclosure provides for a composition comprising: a plurality of nucleic acid-lipid particles, wherein a particle or each particle in the plurality of particles comprises: (a) a nucleic acid; (b) a cationic lipid; (c) a helper lipid comprising at least one acyl chain bearing at least a single unsaturation; (d) a conjugated lipid that inhibits aggregation of particles; and (e) a structural lipid. In some embodiments, the nucleic acid is deoxyribonucleic acid (DNA). In some embodiments, the conjugated lipid is a polyethylene glycol conjugated lipid (PEG-lipid). In some embodiments, the PEG-lipid is PEG-conjugated myristoyl diglyceride (DMG). In some embodiments, the unsaturation of the second acyl chain of the helper lipid is a carbon-8 unsaturation. In some embodiments, the first unsaturated acyl chain of the helper lipid is oleoyl and the second acyl chain of the helper lipid is stearoyl. In some embodiments, the lipid bearing the acyl chain with the carbon-8 unsaturation is a phospholipid. In some embodiments, the helper lipid bears two C16-C18 carbon chains. In some embodiments, the helper lipid comprises a nitrogen-containing head group. In some embodiments, the helper lipid comprises a choline headgroup. In some embodiments, the helper lipid comprises SOPC, POPC, or DOPC. In some embodiments, the lipids and the nucleic acid are present at a ratio of about 20:1 in the composition. In some embodiments, the PEG-lipid comprises at least one hydrocarbon chain with an unsaturation. In some embodiments, the PEG-lipid comprises two hydrocarbon chains with an unsaturation. In some embodiments, the unsaturation of the PEG-lipid occurs at carbon 8 or carbon 4 unsaturation. In some embodiments, the PEG-lipid comprises PEG-conjugated 1,2-dioleoyl-sn-glycero-3-phosphoethanolamine (DOPE). In some embodiments, the PEG-lipid comprises PEG-conjugated 1-stearoyl-2-oleoyl-sn-glycero-3-phosphoethanolamine (SOPE). In some embodiments, the PEG-lipid comprises N-palmitoylsphingosine or C16ceramide. In some embodiments, the cationic lipid is present at a mole % of about at least 30% to about at least 85%. In some embodiments, the helper lipid is present at a mole % of about 8% to about 49.5%. In some embodiments, the PEG-lipid is present at a mole % of about 0.5% to about 8%. In some embodiments, the structural lipid comprises cholesterol. In some embodiments, the cationic lipid comprises DLin-MC3, DLin-KC2, SM102, CKK12, ALC0315, or any combination thereof.

In some aspects, the present disclosure provides for a composition comprising: a plurality of nucleic acid-lipid particles, wherein a particle or each particle in the plurality of particles comprises: (a) DNA; (b) a cationic lipid; (c) a helper lipid; (d) a conjugated lipid that inhibits aggregation of particles, comprising at least one hydrocarbon chain with an unsaturation; and (e) a structural lipid. In some embodiments, the conjugated lipid comprises a polyethylene glycol conjugated lipid (PEG-lipid). In some embodiments, the PEG-lipid comprises two hydrocarbon chains with an unsaturation. In some embodiments, the unsaturation of the PEG-lipid occurs at carbon 8 or carbon 4. In some embodiments, the PEG-lipid comprises PEG-conjugated 1,2-dioleoyl-sn-glycero-3-phosphoethanolamine (DOPE). In some embodiments, the PEG-lipid comprises PEG-conjugated 1-stearoyl-2-oleoyl-sn-glycero-3-phosphoethanolamine (SOPE). In some embodiments, the PEG-lipid comprises N-palmitoylsphingosine or C16ceramide. In some embodiments, the helper lipid comprises at least one hydrocarbon chain bearing an unsaturation. In some embodiments, the unsaturation of the hydrocarbon chain of the helper lipid is a carbon-8 or a carbon-4 unsaturation. In some embodiments, the first unsaturated hydrocarbon chain of the helper lipid is oleoyl and the second hydrocarbon chain of the helper lipid is stearoyl. In some embodiments, the lipid comprising the hydrocarbon chain with the carbon-8 unsaturation is a phospholipid. In some embodiments, the helper lipid comprises two C16-C18 carbon chains. In some embodiments, the helper lipid comprises a nitrogen-containing head group. In some embodiments, the helper lipid comprises a choline headgroup. In some embodiments, the helper lipid comprises SOPC, POPC, or DOPC. In some embodiments, the lipids and the nucleic acid are present at a ratio of about 20:1 in the composition. In some embodiments, the composition comprises a plurality of conjugated lipids that inhibit aggregation of particles, (i) a first conjugated lipid bearing at least one acyl chain with a carbon-8 unsaturation or a carbon-4 unsaturation; and (ii) a second conjugated lipid bearing two saturated C18 acyl chains. In some embodiments, the first conjugated lipid is SOPE, DOPE, C16ceramide, C17ceramide, C18ceramide, or N-palmitoylsphingosine. In some embodiments, the second conjugated lipid is PEG conjugated distearoyl-rac-glycerol (DSG).

In some aspects, the present disclosure provides for a composition comprising: a plurality of nucleic acid-lipid particles, wherein a particle or each particle in the plurality of particles comprises: (a) a nucleic acid; (b) a cationic lipid; (c) a helper lipid; (d) a conjugated lipid that inhibits aggregation of particles; and (e) a carbon-5 saturated cholesterol derivative or a carbon-24 alkyl-substituted cholesterol derivative. In some embodiments, a particle or each particle in the plurality of particles comprises a carbon-5 saturated cholesterol derivative, wherein the carbon-5 saturated cholesterol derivative is a carbon-24 alkyl-substituted cholesterol derivative. In some embodiments, the carbon-5 saturated cholesterol derivative comprises stigmastanol (5α-Stigmastan-3β-ol). In some embodiments, the carbon-5 saturated cholesterol derivative is according to:

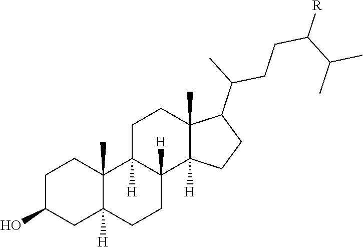

wherein: R is present or absent and is substituted or unsubstituted C₁-C₈ alkyl. In some embodiments, a particle or each particle in the plurality of particles comprises the carbon-24 alkyl-substituted cholesterol derivative, wherein the carbon-24 alkyl-substituted cholesterol derivative is a carbon-5 unsaturated cholesterol derivative. In some embodiments, the carbon-24 alkyl-substituted cholesterol derivative comprises beta-sitosterol (Stigmast-5-en-3β-ol). In some embodiments, the carbon-24 alkyl-substituted cholesterol derivative is according to

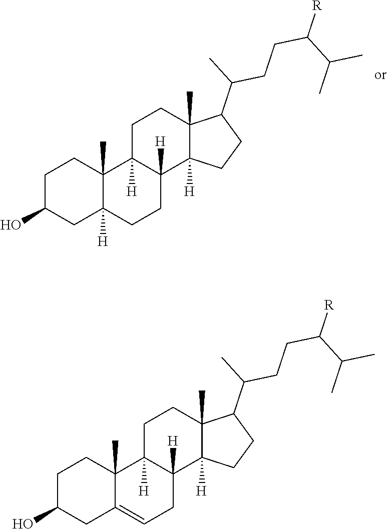

wherein: R is substituted or unsubstituted C₁-C₈ alkyl. In some embodiments, a particle or each particle in the plurality of particles comprises: (i) cholesterol; and (ii) the carbon-5 saturated cholesterol derivative or the carbon-24 alkyl-substituted cholesterol derivative. In some embodiments, a particle or each particle in the plurality of particles comprises from about a 1:9 ratio of (i) to (ii) to about a 9:1 ratio of (i) to (ii). In some embodiments, a particle or each particle in the plurality of particles comprises about a 1:1 ratio of (i) to (ii). In some embodiments, a particle or each particle in the plurality of particles comprises a carbon-5 saturated cholesterol derivative and a carbon-24 alkyl-substituted cholesterol derivative. In some embodiments, the carbon-5 saturated cholesterol derivative is stigmastanol (5α-Stigmastan-3β-ol) and the carbon-24 alkyl-substituted cholesterol derivative is beta-sitosterol (Stigmast-5-en-3β-ol). In some embodiments, the helper lipid comprises at least one acyl chain bearing an unsaturation. In some embodiments, the PEG-lipid comprises at least one hydrocarbon chain with an unsaturation.

In some aspects, the present disclosure provides for a method of delivering DNA to a mammalian cell, comprising: contacting a mammalian cell with any of the compositions described herein. In some embodiments, the mammalian cell is a tumor cell. In some embodiments, the contacting comprises intravenous administration to a subject, wherein the mammalian cell is in the subject. In some embodiments, the delivering to the tumor cell is selective for the tumor cell over a lung, liver, or spleen cell.

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a conjugated lipid that inhibits aggregation of particles in an amount greater than 2 mol % of the total lipid present in the particle; and (e) a structural lipid. In some embodiments, a particle or each particle in the plurality of particles comprises the conjugated lipid in an amount no more than about 15, 12.5, 10, 7.5, 5.0, 4.75, 4.5, 4.0, 3.75, 3.5, 3.25, or 3 mol % of the total lipid present in the particle. In some embodiments, a particle or each particle in the plurality of particles comprises the conjugated lipid in an amount from 2 mol % to 3 mol %. In some embodiments, a particle or each particle in the plurality of particles comprises about 3 mol % of the conjugated lipid. In some embodiments, the conjugated lipid is a PEG-lipid. In some embodiments, the conjugated lipid is a glycerol or succinate derivative bearing two C16-C18 hydrocarbon chains. In some embodiments, the PEG-lipid comprises PEG-chain of about 400 to about 5000 Daltons in molecular weight. In some embodiments, the conjugated lipid is DSG-PEG2000. In some embodiments, the helper lipid comprises at least one acyl chain bearing an unsaturation. In some embodiments, the unsaturation of the second acyl chain of the helper lipid is a carbon-8 unsaturation. In some embodiments, the first unsaturated acyl chain of the helper lipid is oleoyl and the second acyl chain of the helper lipid is stearoyl. In some embodiments, the lipid bearing the acyl chain with the carbon-8 unsaturation is a phospholipid. In some embodiments, the helper lipid bears two C16-C18 carbon chains. In some embodiments, the helper lipid comprises a nitrogen-containing head group. In some embodiments, the helper lipid comprises a choline headgroup. In some embodiments, the helper lipid comprises SOPC (1-stearoyl-2-oleoyl-sn-glycero-3-phosphocholine), POPC (1-palmitoyl-2-oleoyl-glycero-3-phosphocholine), or DOPC (1,2-dioleoyl-sn-glycero-3-phosphocholine). In some embodiments, the structural lipid comprises a carbon-5 saturated cholesterol derivative. In some embodiments, the carbon-5 saturated cholesterol derivative is a carbon-24 alkyl-substituted cholesterol derivative. In some embodiments, the carbon-5 saturated cholesterol derivative comprises stigmastanol (5α-Stigmastan-3β-ol). In some embodiments, the carbon-5 saturated cholesterol derivative is according to:

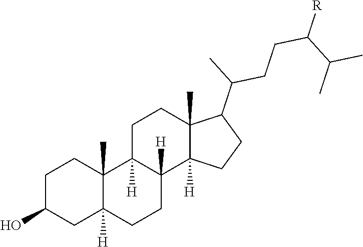

wherein: R is substituted or unsubstituted C₁-C₈ alkyl. In some embodiments, the structural lipid comprises a carbon-24 alkyl-substituted cholesterol derivative. In some embodiments, the carbon-24 alkyl-substituted cholesterol derivative is according to H or

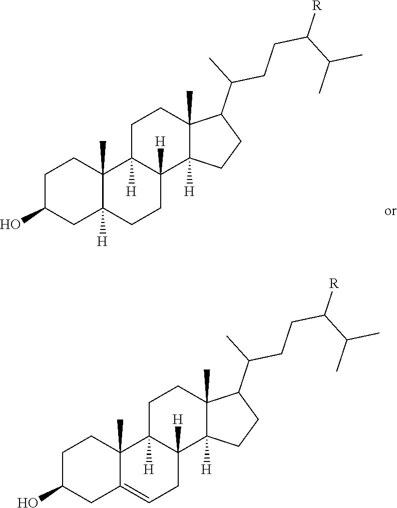

wherein: R is substituted or unsubstituted C₁-C₈ alkyl. In some embodiments, the carbon-24 alkyl-substituted cholesterol derivative comprises beta-sitosterol (Stigmast-5-en-3β-ol). In some embodiments, a particle or each particle in the plurality of particles comprises: (i) cholesterol; and (ii) the carbon-5 saturated cholesterol derivative or the carbon-24 alkyl-substituted cholesterol derivative. In some embodiments, a particle or each particle in the plurality of particles comprises from about a 1:9 ratio of (i) to (ii) to about a 9:1 ratio of (i) to (ii). In some embodiments, a particle or each particle in the plurality of particles comprises about a 1:1 ratio of (i) to (ii).

In some aspects, the present disclosure provides for a method of delivering DNA to a mammalian cell with reduced toxicity, comprising: contacting a mammalian cell with any of the compositions described herein. In some embodiments, the toxicity is assessed by body weight loss, liver enzymes levels, blood lymphocyte levels, serum cytokine levels, or any combination thereof. In some embodiments, the toxicity is assessed by comparison to a reference composition. In some embodiments, the mammalian cell is a tumor cell. In some embodiments, the contacting comprises intravenous administration to a subject, wherein the mammalian cell is in the subject. In some embodiments, the delivering to the tumor cell is selective for the tumor cell over a lung, liver, or spleen cell.

In some aspects, the present disclosure provides for a method of manufacturing a lipid nanoparticle, comprising combining in the solution the components of any of the compositions described herein.

In some aspects, the present disclosure provides for a method of manufacturing a lipid nanoparticle, comprising combining in solution (a) DNA; (b) a cationic lipid; (c) a helper lipid comprising at least one acyl chain bearing an unsaturation; (d) a conjugated lipid that inhibits aggregation of particles; and (e) a structural lipid.

In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles, comprising at least one acyl chain with an unsaturation; and (e) a structural lipid.

In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles; and (e) a carbon-5 saturated cholesterol derivative or a carbon-24 alkyl cholesterol derivative.

In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles in an amount greater than 2 mol % of the total lipid present in the particle; and (e) a structural lipid. In some embodiments, the DNA comprises a DNA vector.

In some aspects, the present disclosure provides for a composition comprising: a plurality of nucleic acid-lipid particles, wherein a particle or each particle in the plurality of particles comprises: (a) DNA; (b) a cationic lipid at a positive amount of not more than about 49 mol % of the total lipid present in the particle; (c) a helper lipid; (d) a conjugated lipid that inhibits aggregation of particles; and (e) a structural lipid. In some embodiments, a particle or each particle in the plurality of particles comprises the cationic lipid at a positive amount of not more than about 45 mol %, 40 mol %, 35 mol %, 30 mol %, 25 mol %, or 20 mol % of the total lipid present in the particle. In some embodiments, a particle or each particle in the plurality of particles comprises the cationic lipid at an amount of about 20 mol % to about 49% of the total lipid present in the particle. In some embodiments, a particle or each particle in the plurality of particles comprises the cationic lipid at an amount of about 20 mol % to about 35 mol % of the total lipid present in the particle. In some embodiments, a particle or each particle in the plurality of particles comprises the cationic lipid at an amount of about 35 mol % of the total lipid present in the particle. In some embodiments, the conjugated lipid is a polyethylene glycol conjugated lipid (PEG-lipid). In some embodiments, the conjugated lipid is a glycerol or succinate derivative bearing two C16-C18 hydrocarbon chains. In some embodiments, the PEG-lipid comprises PEG-chain of about 400 to about 5000 Daltons in molecular weight. In some embodiments, the PEG-lipid comprises PEG-conjugated myristoyl diglyceride (DMG). In some embodiments, the PEG-lipid comprises PEG-conjugated distearoyl-rac-glycerol (DSG). In some embodiments, the helper lipid comprises at least one acyl chain bearing an unsaturation. In some embodiments, the unsaturation of the second acyl chain of the helper lipid is a carbon-8 unsaturation. In some embodiments, the lipid bearing the acyl chain with the carbon-8 unsaturation is a phospholipid. In some embodiments, the helper lipid further bears two C16-C18 carbon chains. In some embodiments, the helper lipid further comprises a choline headgroup. In some embodiments, the helper lipid further comprises SOPC, POPC, or DOPC. In some embodiments, the PEG-lipid further comprises at least one acyl chain with an unsaturation. In some embodiments, the PEG-lipid further comprises two acyl chains with an unsaturation. In some embodiments, the unsaturation of the PEG-lipid occurs at carbon 8. In some embodiments, the PEG-lipid further comprises PEG-conjugated 1,2-dioleoyl-sn-glycero-3-phosphoethanolamine (DOPE). In some embodiments, the PEG-lipid further comprises PEG-conjugated 1-stearoyl-2-oleoyl-sn-glycero-3-phosphoethanolamine (SOPE). In some embodiments, the structural lipid further comprises cholesterol. In some embodiments, the cationic lipid further comprises DLin-MC3, DLin-KC2, SM102, CKK12, ALC0315, or any combination thereof. In some embodiments, the structural lipid further comprises a carbon-5 saturated cholesterol derivative. In some embodiments, the carbon-5 saturated cholesterol derivative is a carbon-24 alkyl-substituted cholesterol derivative. In some embodiments, the carbon-5 saturated cholesterol derivative further comprises stigmastanol (5α-Stigmastan-3β-ol). In some embodiments, the carbon-5 saturated cholesterol derivative is according to:

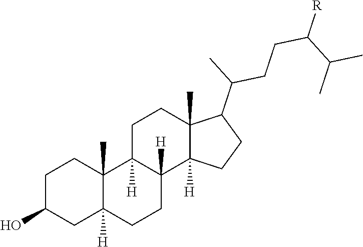

wherein: R is substituted or unsubstituted C₁-C₈ alkyl. In some embodiments, the structural lipid further comprises a carbon-24 alkyl-substituted cholesterol derivative. In some embodiments, wherein the carbon-24 alkyl-substituted cholesterol derivative is according to

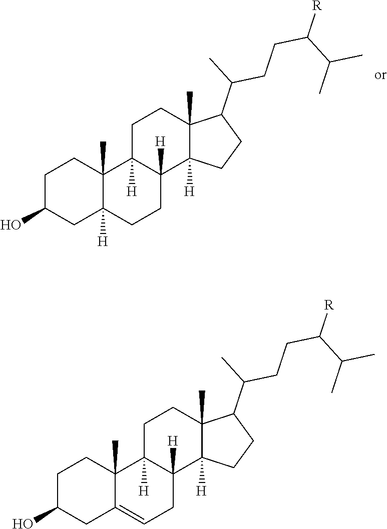

wherein: R is substituted or unsubstituted C₁-C₈ alkyl. In some embodiments, the carbon-24 alkyl-substituted cholesterol derivative comprises beta-sitosterol (Stigmast-5-en-3β-ol). In some embodiments, a particle or each particle in the plurality of particles further comprises: (i) cholesterol; and (ii) the carbon-5 saturated cholesterol derivative or the carbon-24 alkyl-substituted cholesterol derivative. In some embodiments, a particle or each particle in the plurality of particles comprises from about a 1:9 ratio of (i) to (ii) to about a 9:1 ratio of (i) to (ii). In some embodiments, a particle or each particle in the plurality of particles comprises about a 1:1 ratio of (i) to (ii).

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a first conjugated lipid and a second conjugated lipid that inhibit aggregation of particles, wherein: (i) the first conjugated lipid comprises ceramide or ethanolamine; and (ii) the second conjugated lipid does not comprise a head group, or comprises choline as a head group; and (e) a structural lipid. In some embodiments, the first or the second conjugated lipid comprises a polyethylene glycol conjugated lipid (PEG-lipid). In some embodiments, the first conjugated lipid comprises at least one hydrocarbon with an unsaturation. In some embodiments, the unsaturation of the PEG-lipid occurs at carbon 8 or carbon 4. In some embodiments, first conjugated lipid comprises PEG-conjugated 1,2-dioleoyl-sn-glycero-3-phosphoethanolamine (DOPE). In some embodiments, the first conjugated lipid comprises PEG-conjugated 1-stearoyl-2-oleoyl-sn-glycero-3-phosphoethanolamine (SOPE). In some embodiments, the first conjugated lipid comprises N-palmitoylsphingosine or C16ceramide. In some embodiments, the helper lipid comprises at least one acyl chain bearing an unsaturation. In some embodiments, the unsaturation of the acyl chain of the helper lipid is a carbon-8 unsaturation. In some embodiments, the first conjugated lipid bears at least one hydrocarbon chain with a carbon-8 unsaturation or a carbon-4 unsaturation and the second conjugated lipid bears two saturated C16-C18 acyl chains. In some embodiments, the second conjugated lipid is PEG conjugated distearoyl-rac-glycerol (DSG).

In some aspects, the present disclosure provides for a method of delivering DNA to a mammalian cell, comprising: contacting a mammalian cell with any of the compositions described herein. In some embodiments, the mammalian cell is a tumor cell. In some embodiments, the contacting comprises intravenous administration to a subject, wherein the mammalian cell is in the subject. In some embodiments, the delivering to the tumor cell is selective for the tumor cell over a lung, liver, or spleen cell. In some embodiments, the composition increases a lifetime of the DNA in plasma by a factor of at least 10, 10², 10³, 10⁴, 10⁵, or 10⁶ versus a reference composition. In some embodiments, the composition increases delivery of the DNA to a tumor cell by a factor of at least 10, 10², 10³, 10⁴, or more versus a reference composition. In some embodiments, the composition decreases a toxicity of the DNA or the particles to the mammalian cell or the subject by at least 5%, 10%, 15%, 20%, 25%, 30%, 35%, 40%, 45%, 50%, 55%, 60%, 65%, 70%, 75%, 80%, 85%, 90%, 95%, 98%, or 99% compared to a reference composition. In some embodiments, the composition decreases a toxicity of the DNA or the particles to the mammalian cell or the subject by at a factor of at least 10, 10², 10³, 10⁴, 10⁵, or 10⁶ compared to a reference composition.

In some aspects, the present disclosure provides for a composition comprising: a plurality of nucleic acid-lipid particles, wherein a particle or each particle in the plurality of particles comprises: (a) DNA; (b) a cationic lipid at a positive amount of not more than about 49 mol % of the total lipid present in the particle; (c) a helper lipid comprising at least one acyl chain bearing a single unsaturation; (d) (i) a conjugated lipid that inhibits aggregation of particles, comprising at least one hydrocarbon chain with an unsaturation; and/or (ii) a conjugated lipid that inhibits aggregation of particles in an amount greater than 2 mol % of the total lipid present in the particle; and/or (iii) a first conjugated lipid and a second conjugated lipid that inhibit aggregation of particles, wherein the first conjugated lipid comprises ceramide or ethanolamine and the second conjugated lipid does not comprise ahead group, or comprises choline as ahead group; and (e) a structural lipid. In some embodiments, the composition comprises a conjugated lipid that inhibits aggregation of particles, comprising at least one hydrocarbon chain with an unsaturation. In some embodiments, the composition comprises a conjugated lipid that inhibits aggregation of particles in an amount greater than 2 mol % of the total lipid present in the particle. In some embodiments, the composition comprises a first conjugated lipid and a second conjugated lipid that inhibit aggregation of particles, wherein the first conjugated lipid comprises ceramide or ethanolamine and the second conjugated lipid does not comprise a head group, or comprises choline as a head group. In some embodiments, the structural lipid comprises a carbon-5 saturated cholesterol derivative or a carbon-24 alkyl cholesterol derivative.

In some aspects, the present disclosure provides for a composition comprising: a plurality of nucleic acid-lipid particles, wherein each particle in the plurality of particles comprises: (a) a nucleic acid; (b) a cationic lipid; (c) a helper lipid comprising at least one acyl chain bearing a single unsaturation; (d) a conjugated lipid that inhibits aggregation of particles; and (e) a structural lipid. In some embodiments, the nucleic acid is deoxyribonucleic acid (DNA). In some embodiments, the conjugated lipid is a polyethylene glycol conjugated lipid (PEG-lipid). In some embodiments, the PEG-lipid is PEG-conjugated myristoyl diglyceride (DMG). In some embodiments, the unsaturation of the second acyl chain of the helper lipid is a carbon-8 unsaturation. In some embodiments, the first unsaturated acyl chain of the helper lipid is oleoyl and the second acyl chain of the helper lipid is stearoyl. In some embodiments, the lipid bearing the acyl chain with the carbon-8 unsaturation is a phospholipid. In some embodiments, the helper lipid bears two C16-C18 carbon chains. In some embodiments, the helper lipid comprises a cationic head group. In some embodiments, the helper lipid comprises a choline headgroup. In some embodiments, the helper lipid comprises SOPC, POPC, or DOPC. In some embodiments, the lipids and the nucleic acid are present at a ratio of about 20:1 in the composition. In some embodiments, the PEG-lipid comprises at least one acyl chain with an unsaturation. In some embodiments, the PEG-lipid comprises two acyl chains with an unsaturation. In some embodiments, the unsaturation of the PEG-lipid occurs at carbon 8. In some embodiments, the PEG-lipid comprises PEG-conjugated 1,2-dioleoyl-sn-glycero-3-phosphoethanolamine (DOPE). In some embodiments, the PEG-lipid comprises PEG-conjugated 1-stearoyl-2-oleoyl-sn-glycero-3-phosphoethanolamine (SOPE). In some embodiments, the cationic lipid is present at a mole % of about at least 30% to about at least 85%. In some embodiments, the helper lipid is present at a mole % of about 8% to about 49.5%. In some embodiments, the PEG-lipid is present at a mole % of about 0.5% to about 8%. In some embodiments, the structural lipid comprises cholesterol. In some embodiments, the cationic lipid comprises DLin-MC3, DLin-KC2, SM102, CKK-E12, ALC0315, or any combination thereof.

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a conjugated lipid that inhibits aggregation of particles, comprising at least one acyl chain with an unsaturation; and (e) a sterol. In some embodiments, the conjugated lipid comprises a polyethylene glycol conjugated lipid (PEG-lipid). In some embodiments, the PEG-lipid comprises two acyl chains with an unsaturation. In some embodiments, the unsaturation of the PEG-lipid occurs at carbon 8. In some embodiments, the PEG-lipid comprises PEG-conjugated 1,2-dioleoyl-sn-glycero-3-phosphoethanolamine (DOPE). In some embodiments, the PEG-lipid comprises PEG-conjugated 1-stearoyl-2-oleoyl-sn-glycero-3-phosphoethanolamine (SOPE). In some embodiments, the helper lipid comprises at least one acyl chain bearing an unsaturation. In some embodiments, the unsaturation of the second acyl chain of the helper lipid is a carbon-8 unsaturation. In some embodiments, the first unsaturated acyl chain of the helper lipid is oleoyl and the second acyl chain of the helper lipid is stearoyl. In some embodiments, the lipid comprising the acyl chain with the carbon-8 unsaturation is a phospholipid. In some embodiments, the helper lipid comprises two C16-C18 carbon chains. In some embodiments, the helper lipid comprises a cationic head group. In some embodiments, the helper lipid comprises a choline headgroup. In some embodiments, the helper lipid comprises SOPC, POPC, or DOPC. In some embodiments, the lipids and the nucleic acid are present at a ratio of about 20:1 in the composition. In some embodiments, the composition comprises a plurality of conjugated lipids that inhibit aggregation of particles, (i) a first conjugated lipid bearing at least one acyl chain with a carbon-8 unsaturation; and (ii) a second conjugated lipid bearing two saturated C18 acyl chains. In some embodiments, the first conjugated lipid is SOPE or DOPE. In some embodiments, the second conjugated lipid is PEG conjugated distearoyl-rac-glycerol (DSG).

In some aspects, the present disclosure provides for a composition comprising: a plurality of nucleic acid-lipid particles, wherein each particle in the plurality of particles comprises: (a) a nucleic acid; (b) a cationic lipid; (c) a helper lipid; (d) a conjugated lipid that inhibits aggregation of particles; and (e) a carbon-5 saturated cholesterol derivative or a carbon-24 alkyl-substituted cholesterol derivative. In some embodiments, the carbon-5 saturated cholesterol derivative is a carbon-24 alkyl-substituted cholesterol derivative. In some embodiments, the carbon-5 saturated cholesterol derivative comprises stigmastanol (5α-Stigmastan-3β-ol). In some embodiments, the carbon-5 saturated cholesterol derivative is according to:

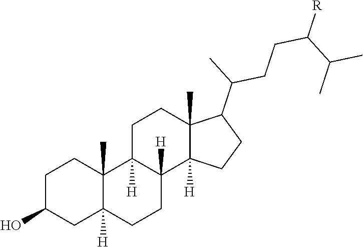

wherein:
R is selected from substituted or unsubstituted C₁-C₈ alkyl. In some embodiments, the helper lipid comprises at least one acyl chain bearing an unsaturation. In some embodiments, the PEG-lipid comprises at least one acyl chain with an unsaturation.

In some aspects, the present disclosure provides for a method of delivering DNA to a mammalian cell, comprising: contacting a mammalian cell with any of the compositions described herein. In some embodiments, the mammalian cell is a tumor cell.

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a conjugated lipid that inhibits aggregation of particles comprising about 3 mole % of the total lipid present in the particle; and (e) a sterol. In some embodiments, the conjugated lipid is a PEG-lipid. In some embodiments, the conjugated lipid is a glycerol derivative bearing two saturated C18 acyl chains. In some embodiments, the PEG-lipid comprises PEG-chain of about 2000 Daltons in molecular weight. In some embodiments, the conjugated lipid is DSG-PEG2000. In some embodiments, the helper lipid comprises at least one acyl chain bearing an unsaturation. In some embodiments, the unsaturation of the second acyl chain of the helper lipid is a carbon-8 unsaturation. In some embodiments, the first unsaturated acyl chain of the helper lipid is oleoyl and the second acyl chain of the helper lipid is stearoyl. In some embodiments, the lipid bearing the acyl chain with the carbon-8 unsaturation is a phospholipid. In some embodiments, the helper lipid bears two C16-C18 carbon chains. In some embodiments, the helper lipid comprises a cationic head group. In some embodiments, the helper lipid comprises a choline headgroup. In some embodiments, the helper lipid comprises SOPC, POPC, or DOPC. In some embodiments, the sterol comprises a carbon-5 saturated cholesterol derivative. In some embodiments, the carbon-5 saturated cholesterol derivative is a carbon-24 alkyl-substituted cholesterol derivative. In some embodiments, the carbon-5 saturated cholesterol derivative comprises stigmastanol (5α-Stigmastan-3β-ol). In some embodiments, the carbon-5 saturated cholesterol derivative is according to:

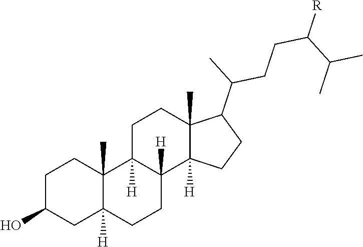

wherein: R is selected from substituted or unsubstituted C₁-C₈ alkyl.

In some aspects, the present disclosure provides for a method of delivering DNA to a mammalian cell with reduced toxicity, comprising: contacting a mammalian cell with an LNP composition comprising a conjugated lipid that inhibits aggregation of particles comprising about 3 mole % of the total lipid present in the particle. In some embodiments, the toxicity is assessed by body weight loss, liver enzymes levels, blood lymphocyte levels, serum cytokine levels, or any combination thereof. In some embodiments, the mammalian cell is a tumor cell.

In some aspects, the present disclosure provides for a method of manufacturing a lipid nanoparticle, comprising combining in solution (a) DNA; (b) a cationic lipid; (c) a helper lipid comprising at least one acyl chain bearing an unsaturation; (d) a conjugated lipid that inhibits aggregation of particles; and (e) a structural lipid. In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles, comprising at least one acyl chain with an unsaturation; and (e) a sterol. In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles; and (e) a carbon-5 saturated cholesterol derivative. In some aspects, the present disclosure provides for a method of manufacturing a lipid nanoparticle, comprising combining in solution (a) DNA; (b) a cationic lipid; (c) a helper lipid; (d) a conjugated lipid that inhibits aggregation of particles comprising about 3 mole % of the total lipid present in the particle; and (e) a sterol. In some embodiments, the DNA comprises a DNA vector.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic illustrating an example lipid nanoparticle (LNP), comprising PEG-lipid, cationic lipid, helper lipid, cholesterol, and nucleic acid (e.g., DNA or RNA).

FIG. 1B shows example chemical structures of helper lipids (e.g., carbon-8 unsaturated phospholipids), including SOPC (18:0-18:1 PC, 1-stearoyl-2-oleoyl-sn-glycero-3-phosphocholine), DOPE (18:1 (Δ9-Cis) PE, 1,2-dioleoyl-sn-glycero-3-phosphoethanolamine), POPC (16:0-18:1 PC, 1-palmitoyl-2-oleoyl-glycero-3-phosphocholine), and DOPC (18:1 (Δ9-Cis) PC, 1,2-dioleoyl-sn-glycero-3-phosphocholine) for use with LNP compositions as described herein.

FIG. 1C shows example chemical structures of cationic lipids, including D-Lin-MC3-DMA (MC3, (6Z,9Z,28Z,31Z)-Heptatriaconta-6,9,28,31-tetraen-19-yl 4-(dimethylamino)butanoate) for use with LNP compositions as described herein.

FIG. 1D shows example PEG-lipids, including DMG-PEG 2000 (1,2-dimyristoyl-rac-glycero-3-methoxypolyethylene glycol-2000) for use with LNP compositions as described herein.

FIGS. 2A, 2B, and 2C demonstrate that a phospholipid bearing an acyl (e.g., fatty acyl) chain with a carbon-8 unsaturation (e.g., SOPC) can improve DNA delivery when included in an LNP formulation. FIG. 2A shows a bar graph comparing expression as assessed by bioluminescence imaging of livers of Balb/c mice delivered 20 ug of a DNA nanoplasmid bearing CMV-luciferase by intravenous injection 4 days previously. Conditions shown are vehicle (phosphate buffered saline, “PBS”) with nanoplasmid, an alternative DNA delivery agent (JetPEI according to manufacturer's instructions, “JetPEI”) with nanoplasmid, or nanoplasmid formulated into LNPs included at a mass ration of 10:1 total lipids:DNA with 50 mole % cationic lipid (e.g., Dlin-MC3-DMA), 38.5 mole % cholesterol, 1.5 mole % PEG lipid (e.g., DMG-PEG) and either 10% phospholipid bearing an acyl (e.g., fatty acyl) chain with a carbon-8 unsaturation (“SOPC”) or phospholipid with unsaturated chains (“DSPC”). Carbon-8 unsaturated lipid shows a large enhancement (~15×) in apparent delivery to liver as assessed by luciferase expression when compared to the other conditions. FIG. 2B shows an example luminescence imaging photograph on a similar experiment as that performed in FIG. 2A but comparing LNP formulated using helper phospholipids bearing an acyl (e.g., fatty acyl) chain with a carbon-8 unsaturation (“SOPC”) and helper phospholipids with two carbon-8 unsaturated chains and an ethanolamine head group (“DOPE”) and at day 1 after intravenous injection of the LNPs. FIG. 2C shows the same experiment as FIG. 2C, but in the format of a bar graph comparing DOPE and SOPC conditions.

FIG. 3 demonstrates that a reduction of PEG-lipid concentration in the default LNP formulation further improves delivery of DNA nanoplasmid to liver cells over inclusion of phospholipid with an acyl chain with an unsaturation (e.g., a single unsaturation at carbon-8). Shown is a bar graph of a similar experiment to FIG. 2A formulating CMV-luciferase DNA nanoplasmid into LNPs with 50 mole % cationic lipid (e.g., Dlin-MC3-DMA), 10% helper phospholipid (e.g., SOPC), and either 0.75 mole % PEG lipid such as DMG-PEG (“0.75% PEG”) and 39.25 mole % cholesterol, or 1.5 mole % PEG lipid such as DMG-PEG (“1.5% PEG”) and 38.5% cholesterol, injecting intravenously into Balb/c mice, and performing bioluminescence imaging on the liver at 1 day after injection. The lower PEG condition (“0.75% PEG”) shows significant enhancement (~3×) of DNA delivery compared to the higher PEG condition (“1.5% PEG”).

FIGS. 4A and 4B demonstrate that inclusion of other helper phospholipids bearing an acyl (e.g., fatty acyl) chain with an unsaturation (e.g., a single carbon-8 unsaturation) (DOPC, POPC) also enhance DNA delivery when included in an LNP formulation. FIG. 4A compares the chemical structures of DOPC (18:1 (Δ9-Cis) PC or 1,2-dioleoyl-sn-glycero-3-phosphocholine) and POPC (16:0-18:1 PC or 1-palmitoyl-2-oleoyl-glycero-3-phosphocholine). FIG. 4B shows a bar graph on an in vitro experiment where DNA vectors were formulated into LNPs with 50 mole % cationic lipid (e.g., Dlin-MC3-DMA), 39.25 mole % cholesterol, 0.75 mole % PEG lipid (e.g., DMG-PEG) and either 10% SOPC (“EM-40”), 10% DOPC (“DOPC”), or 10% POPC (“POPC) and exposed to a cultured mammalian cell line (H129). Shown are percentage of cells positive of those live for cells that took up the DNA (“% Cell Uptake”), took up the DNA into their nucleus (“% Nuclear Delivery”), or successfully expressed the vector DNA-encoded reporter protein (“% Transfection”). For the “% Cell Uptake” and “% Nuclear Delivery” measures, DNA vector labeled with Cy5 was used in the LNPs, and uptake into either the cell overall or the nucleus specifically was performed 24 hours after introduction of the LNP by FACS for the Cy5 signal (nuclei measurements were performed on nuclei isolated from cells). For the “% Biomarker Expression” measurement, DNA vector encoding GFP was used in the LNPs, and the cells were assessed for the GFP signal by FACS. Both LNPs formulated with DOPC and POPC showed similar performance in all three measures to those formulated with SOPC.

FIG. 5 demonstrates that LNP formulations using helper phospholipids bearing an acyl (e.g., fatty acyl) chain with an unsaturation (e.g., a single carbon-8 unsaturation) and reduced PEG lipid (e.g., “EM40”) show superior delivery to cultured eukaryotic cells compared to a commercial LNP nucleic acid delivery formulation. Shown is a bar graph of a similar experiment to FIG. 4B where fluorescent protein encoding DNA vector was formulated into JetPEI according to manufacturer's instructions (“JetPEI”), lipofectamine according to manufacturer's instructions (“Lipofectamine”), LNPs with 50 mole % cationic lipid (e.g., Dlin-MC3-DMA), 39.25 mole % cholesterol, 0.75 mole % PEG lipid (e.g., DMG-PEG), and 10% SOPC (“EM40), or LNPs with 50 mole % cationic lipid (e.g., Dlin-MC3-DMA), 39.25 mole % cholesterol, 1.5 mole % PEG lipid (e.g., DMG-PEG), and 10% SOPC (“EM40”), or LNPs with 50 mole % cationic lipid (e.g., Dlin-MC3-DMA), 39.25 mole % cholesterol, 0.75 mole % PEG lipid (e.g., DMG-PEG), and 10% SOPC (“EM40”), or LNPs with 50 mole % cationic lipid (e.g., Dlin-MC3-DMA), 38.5 mole % cholesterol, 1.5 mole % PEG lipid (e.g., DMG-PEG), and 10% DSPC (“Commercial formulation”). Shown are percentage of cells positive of those live for cells that took up the DNA (“% Cell Uptake”), took up the DNA into their nucleus (“% Nuclear Delivery”), or successfully expressed the vector DNA-encoded reporter protein (“% Transfection”). “% Cell Uptake”, “% Nuclear Delivery”, and “% Transfection” was assessed as in FIG. 4B. Notably, the improved formulation (“EM40”, also known as FRM055 herein) shows better performance for transfection versus a commercial formulation or JetPEI.

FIGS. 5A and 5B show a schematic for a combined uptake/expression assay to track both internalization and expression of reporters from LNPs as described herein (e.g., Example 2 and 3).

FIGS. 6A and 6B demonstrate that substituting other cationic lipids (DLin-KC2/“KC2”, “ALC0315”, “SM102”, and “cKKE12”) in the background an LNP formulation comprising a helper phospholipid bearing an acyl (e.g., fatty acyl) chain with a carbon-8 unsaturation largely has equivalent or greater transfection efficiency. FIG. 6A shows a graph of the percentage of GFP positive H1299 cells as measured by flow cytometry for the LNP formulations using the ionizable lipids noted. FIG. 6B shows structures of lipids of interest.

FIG. 7 demonstrates that LNP formulations described herein (e.g., “EM40”, “FRM119”) produce similar or better in vivo delivery tumors of a mouse H1299 xenograft model to JetPEI (“JetPEI”). Shown are graphs of BLI imaging results on mice injected with LNP compositions of DNA vectors driving a BLI reporter. The graphs additionally demonstrate that further surface modification of LNP formulations to incorporate increased concentrations (e.g., 3 mole %) of a PEG-lipid bearing two saturated acyl chains (e.g., C18 acyl chains, “FRM 119”) improves delivery to tumor and lowers delivery to other tissues such as lung, liver, and spleen.

FIG. 8 demonstrates decreased liver and non-tumor expression driven by adding surface modification of LNP formulations described herein. Shown are photographs of BLI imaging of tumor and other organs from mice administered LNPs as in FIG. 6 showing that LNP formulations to incorporate increased concentrations (e.g., 3 mole %) of a PEG-lipid bearing two saturated acyl chains (e.g., C18 acyl chains, “FRM119”) show liver expression near the lower limit of detection compared to compositions bearing decreased concentrations of PEG-lipids with two saturated acyl (e.g., fatty acyl) chains (e.g., C14 acyl chains, “EM40”).

FIGS. 9A and 9B demonstrate that surface modification of LNP formulations described herein (“FRM 119”) results in enhanced levels of LNPs in serum 48 hr after injection into mice, and enhanced levels in tumors. FIG. 9A shows a graph of DNA copies per μL plasma assessed in mice by qPCR 48 hours after intravenous injection. FIG. 9B shows a graph of DNA copies per cell in dissected tumors from corresponding mice 48 hours after administration.

FIGS. 10A and 10B demonstrate that LNP formulations described herein (“FRM 119”, “FRM129”, “FRM131”, “FRM146”, and “FRM156”) can result in selectivity of tumor vs liver targeting. Shown is a graph of BLI imaging of dissected organs 48 hours of injection into a mouse H1299 xenograft model. FIG. 10A shows tissue selectivity for FRM119, FRM 129, and FRM131,while FIG. 10B shows tissue selectivity for FRM119, FRM146, and FRM156.

FIGS. 11A, 11B, 11C, 11D, 11E, and 11F demonstrate that modifying length of lipid chain in the conjugated lipid, PEG linker length of the conjugated lipid, and concentration of conjugated lipid modifies expression and uptake of LNPs, and that compositions having: (a) conjugated lipids with C18 chains combined with (b) increased concentrations of conjugated lipid have improved expression properties in vivo. FIG. 11A shows the effect of lipid tail length on uptake and expression of LNPs as assessed in Example 4. FIG. 11B shows the effect of surface loading on uptake and expression of LNPs as assessed in Example 4. FIG. 11C shows the effect of PEG length on uptake and expression of LNPs as assessed in Example 4. FIG. 11D shows a plot of measurements on different LNP formulations incubated with H1299 cells in vitro; Notation is: LipidTail-PolymerLength-SurfaceLoading-+/−SerumIncubation. The dotted oval represents the hypothesized target profile of a long-circulating LNP. FIG. 11E shows an evaluation of in vivo pharmacokinetics of LNPs, demonstrating that the combination of C18 chain length, 2K PEG length, and 3% PEG-lipid loading improve stability in plasma. (A) The relative position of different LNPs on the Uptake-Expression Correlation Plot. (B) PK analysis of circulating DNA in plasma 48 h after administration. The DNA copies in the plasma were normalized to the PBS-treated groups to enable comparisons across different in vivo experiments. FIG. 11F shows PK analyses of LNPs in tumor tissue demonstrating that C18 lipid tails, in some contexts, can provide improved accumulation in the tumor relative to C14 lipid tails as evaluated in Example 4.

FIG. 12 shows design space to evaluate the effect of saturated lipid-PEGs as evaluated in Example 4.

FIGS. 13A, 13B, and 13C demonstrate that LNP formulations using helper phospholipids bearing an acyl (e.g., fatty acyl) chain with a carbon-8 unsaturation and reduced PEG lipid (e.g., “EM40”) have a superior toxicity profile in terms of body weight loss, liver enzyme levels, and lymphopenia to JetPEI. CD-1 mice were delivered vehicle (PBS), EM40 LNPs at the indicated doses, or JetPEI at the indicated doses. Shown are graphs of body weight (FIG. 13A), liver enzyme levels (ALT, FIG. 13B), and blood lymphocyte levels (FIG. 13C). The results indicate EM-40 at 2-fold higher dose than JetPEI results in similar toxicity.

FIGS. 14A, 14B, 14C show that LNP formulation FRM119 has an improved toxicity profile to EM40. FIG. 14A shows liver enzymes in mice administered the indicated doses of EM40 or FRM119 LNPs bearing DNA, while FIG. 14B shows corresponding levels of IL-6 and FIG. 14C shows corresponding levels of TNF-alpha.

FIG. 15 demonstrates that carbon-24 alkyl-substituted cholesterol derivatives (e.g., beta-sitosterol and stigmastanol), and particularly carbon-5 saturated cholesterol derivatives (e.g., stigmastanol), show improved delivery of DNA in cells when included in an LNP formulation. Shown is a graph of GFP expression by H1299 cells treated with LNPs as assessed by flow cytometry according to the protocol in Example 2 (horizontal line represents mean value). The LNP formulations were prepared with either MC3 or ALC-0315 as the ionizable lipid, DSPC, SOPC, or SOPE as the helper lipid, a mixture of DOPE-PEG1000 and DSG-PEG2000 as the lipid-PEGs, and one of the following sterols: “cholesterol” (Cholest-5-en-3β-ol), “beta-sitosterol”(Stigmast-5-en-3β-ol), or “stigmastanol” (5α-Stigmastan-3β-ol). Sitosterol produced higher GFP expression than cholesterol and stigmastanol produced higher GFP expression than sitosterol and cholesterol.

FIG. 16A demonstrates that flexible surfaces in LNPs can achieve a balance of long circulation and high transfection in vitro. Shown in the left panel is a graph of expression (measured as GFP fluorescence) vs uptake (measured as Cy5 fluorescence) for FRM119, FRM156, FRM182, FRM060, and FRM146 measured in an in-vitro expression/uptake assay as described in Example 4. Shown in the right panel is graph of uptake in cells and nuclei, measured by Cy5 fluorescence, versus time in hours for an equivalent experiment.

FIG. 16B demonstrates that flexible surfaces in LNPs can achieve a balance of long circulation and high transfection in vivo. Shown in the left panel is a graph of DNA copies per microliter plasma versus LNP composition (FRM060, FRM119, or FRM146) for mice administered FRM060, FRM119, or FRM146 compositions bearing reporter DNA as described in Example 5. Shown in the right panel is a graph of DNA copies in the tumor per cell versus LNP composition (FRM060, FRM119, or FRM146) for mice administered FRM060, FRM119, or FRM146 compositions bearing reporter DNA as described in Example 5.

FIG. 16C depicts an example of high-throughput screening that identified several compositions that produced significant increases in GFP expression compared to FRM156 in vitro, resulting in the discovery of formulations FRM214 and FRM225.

FIG. 16D depicts some of the results of the experiment in Example 6. Shown in the left panel is a graph of DNA copies per microliter plasma versus LNP composition (FRM146, FRM156, FRM214, or FRM225) for mice administered FRM146, FRM156, FRM214, or FRM225 compositions bearing reporter DNA as described in Example 6. Shown in the right panel is a graph of bioluminescent intensity of tumors transfected with the named LNP formulations (FRM146, FRM156, FRM214, or FRM225) bearing reporter DNA as described in Example 6.

FIG. 16E depicts the results of the experiment in Example 7. Shown is a chart of tumor BLI above background for each LNP (FRM055, FRM119, FRM146, or FRM156) bearing reporter DNA injected into mice as described in Example 7. Each bar represents an individual experiment with at least 5 mice, demonstrating that the trends were consistent across repeated experiments.

FIG. 16F depicts some of the results of the experiment in Example 7. Shown are representative luminescent images of dissected organs (tumor, lung, liver) from mice transfected as in FIG. 16E and Example 7.

FIG. 16G depicts some of the results of the experiment in Example 8. Shown are graphs of body weight or serum aspartate aminotransferase (AST) versus time after administration of FRM146 or FRM055 to mice as described in Example 8.

FIG. 16H depicts some of the results of the experiment in Example 8. Shown are graphs of serum tumor necrosis factor alpha (TNF-alpha) or Monocyte chemoattractant protein-1 (MCP1) versus time after administration of FRM146 or FRM055 to mice as described in Example 8.

FIG. 16I depicts some of the results of the experiment in Example 8. Shown is a graph of serum Interleukin 6 (IL6) versus time after administration of FRM146 or FRM055 to mice as described in Example 8.

FIG. 16J depicts some of the results of the experiment in Example 9. Shown are representative BLI images of mice dosed with the named LNPs (FRM055, FRM146, or FRM156) bearing reporters 48 hours after intravenous dosage as described in Example 9.

FIG. 16K depicts some of the results of the experiment in Example 9. Shown is a graph of BLI intensity for individual organs after gross necropsy for mice dosed with the named LNPs (FRM055, FRM146, or FRM156) bearing reporters 48 hours after intravenous dosage as described in Example 9.

FIG. 17A depicts some of the results of the experiment in Example 10. Shown is a graph of bioluminescent intensity (BLI) of dissected organs 48 hours after injection of the named LNPs (FRM136 or FRM 237) bearing reporters into a mouse H1299 and H1373 xenograft model as described in Example 10.

FIG. 17B depicts some of the results of the experiment in Example 10. Shown is a graph of the percentage (%) remaining injected dose of FRM237, FRM238, or FRM239 reporter bearing LNPs in the plasma at 48 hours after injection in mice.

FIG. 17C depicts some of the results of the experiment in Example 10. Shown is a summary schematic depicting the different LNP features in the formulations depicted (FRM 238, FRM119, FRM156, FRM239, FRM237, FRM146, and EM40/FRM055) versus the % remaining injected dose in the plasma of mice at 48 hours as described in Example 10.

FIG. 18A demonstrates that carbon-24 alkyl-substituted cholesterol derivatives (beta-sitosterol) show improved delivery of DNA to tumors in mice when included in an LNP formulation. Shown is a graph of BLI imaging of dissected tumor 48 hours post intravenous administration of LNP to a murine H1299 or H1373 xenograft model at the same relative dose of DNA. The LNP formulations were prepared with MC3 as the ionizable lipid, SOPC as the helper lipid, a mixture of DOPE-PEG1000 and DSG-PEG2000 as the lipid-PEGs and either “cholesterol” (Cholest-5-en-3β-ol), “beta-sitosterol” (Stigmast-5-en-3β-ol) or a mixture thereof.

FIG. 18B demonstrates that carbon-5 saturated cholesterol derivatives (stigmastanol) show improved delivery of DNA to tumors in mice when included in an LNP formulation as described in Example 11. Shown is a graph of BLI imaging of dissected tumor 48 hours post intravenous administration of LNP to a murine H1299 or H1373 xenograft model at the same relative dose of DNA. The LNP formulations were prepared with MC3 as the ionizable lipid, SOPC as the helper lipid, a mixture of DOPE-PEG1000 and DSG-PEG2000 as the lipid-PEGs and either “cholesterol” (Cholest-5-en-3β-ol), “stigmastanol” (5α-Stigmastan-3β-ol) or a mixture thereof.

FIG. 18C demonstrates that fully replacing the cholesterol in a LNP formulation with a cholesterol derivative such as stigmastanol can be used to modulate the circulation profile of LNPs as described in Example 11. Specifically, addition of increasing amounts of sterol derivative lead to shorter circulation profiles. Shown is the copies of Earli DNA present in plasma 48-hours post dose (an indirect measure for the amount of DNA-LNP present in circulation) determined by qPCR analysis.

FIG. 19 demonstrates that changing the molar ratio of ionizable lipid from 50 mol % to 35 mol % leads to a reduction in the toxicity of the drug product as evidenced by reduction in the body weight loss as described in Example 12. Shown are plots of the mouse bodyweight changes (relative to pre-dosing weight) for mice that had DNA-LNP intravenously administered at 1.4 mpk (top graph) or 0.7 mpk (lower graph). Body weight loss is an important metric that indicates the tolerability of the drug product with more toxic formulations leading to greater bodyweight loss.

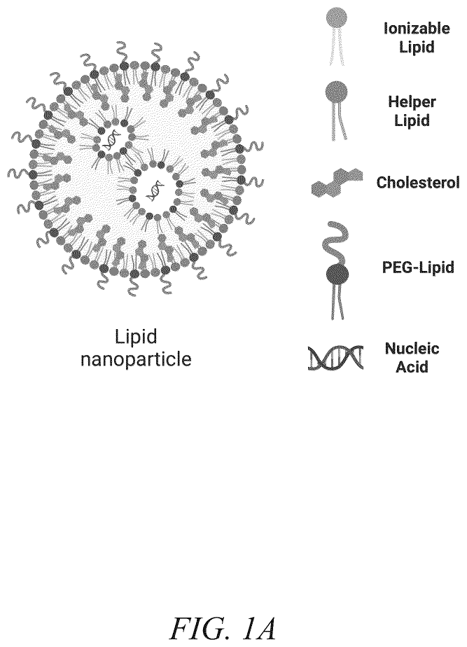

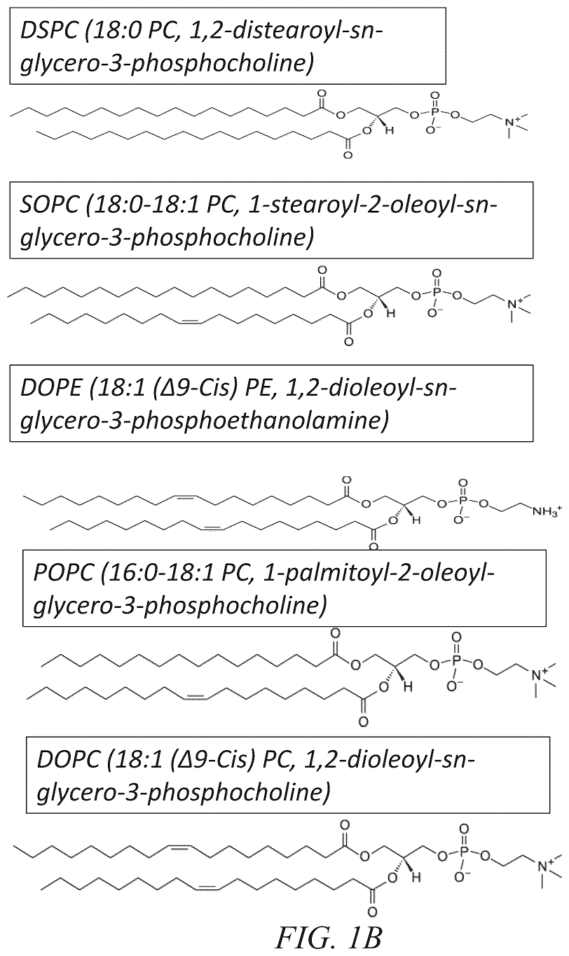

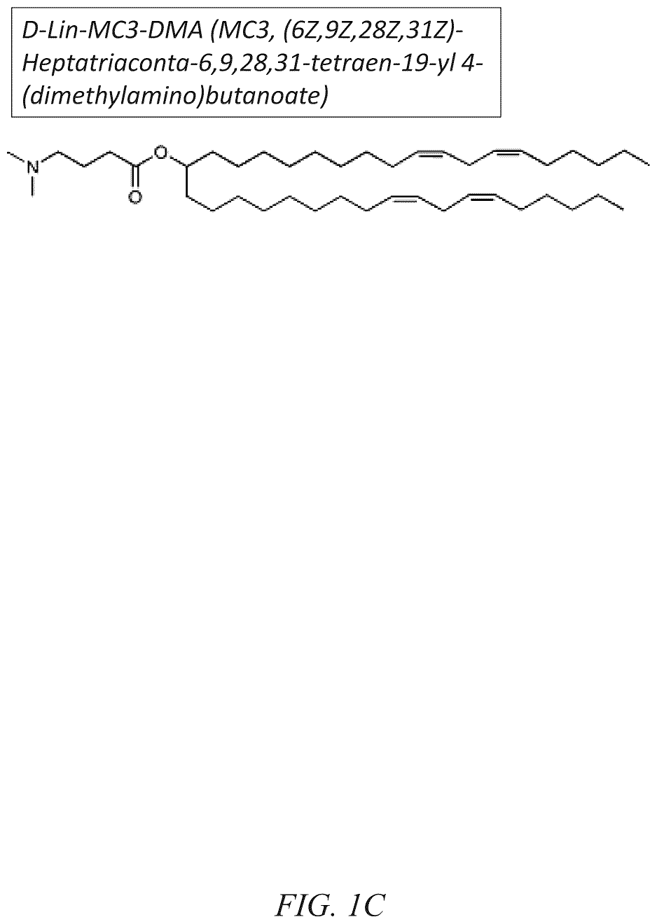

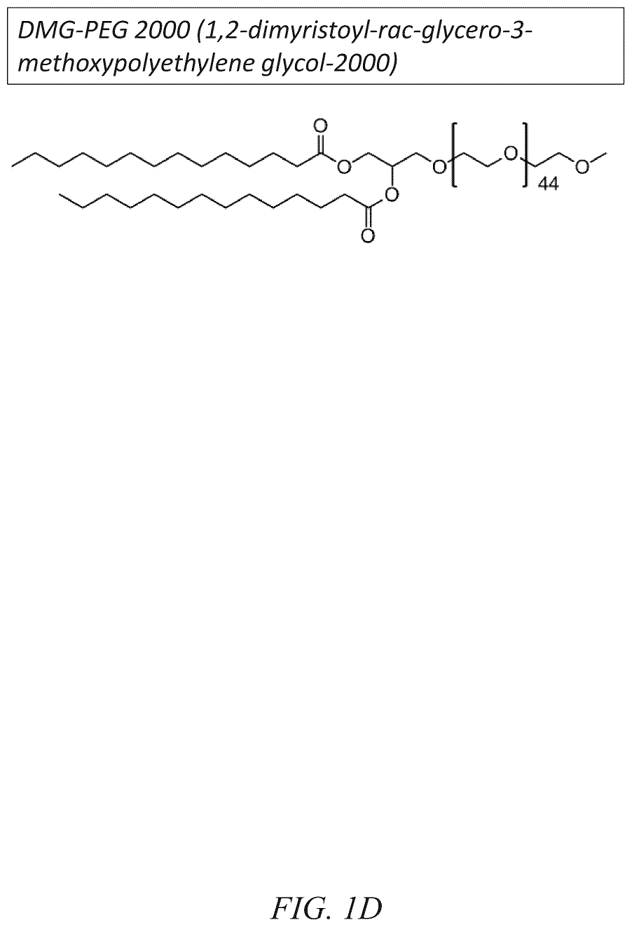

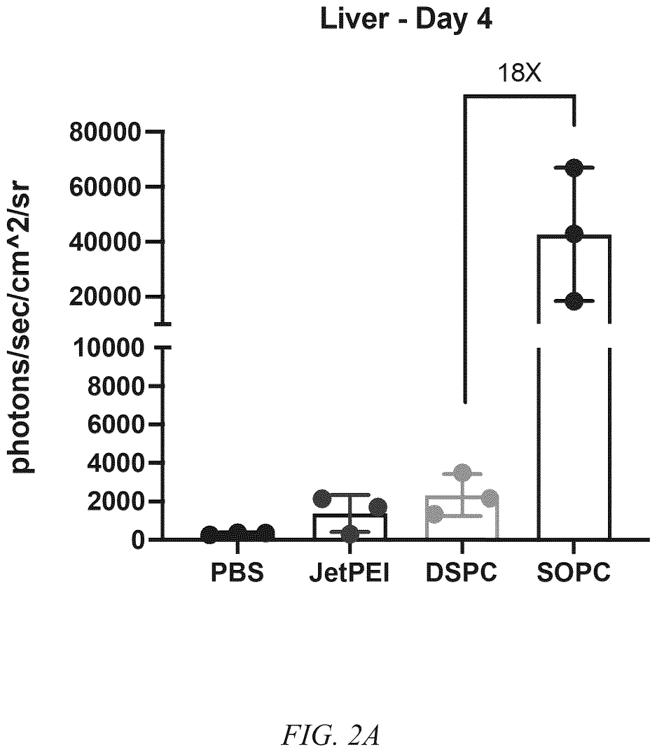

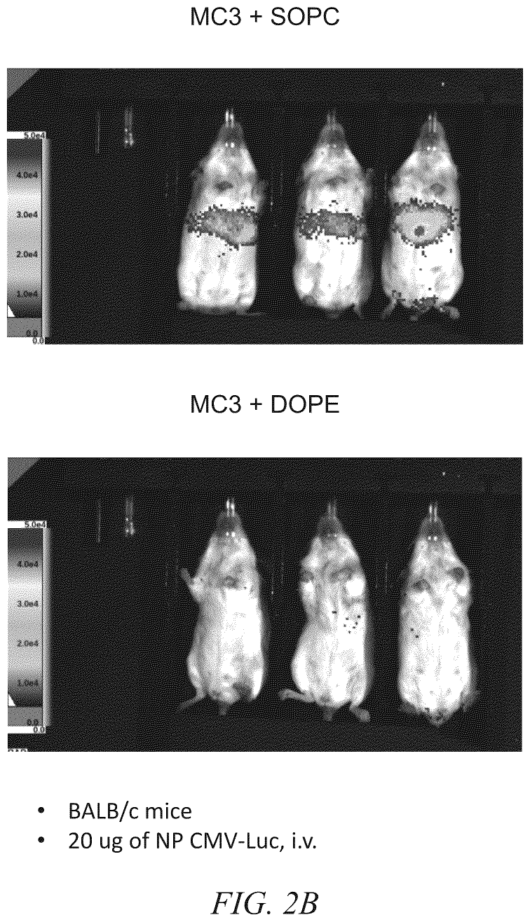

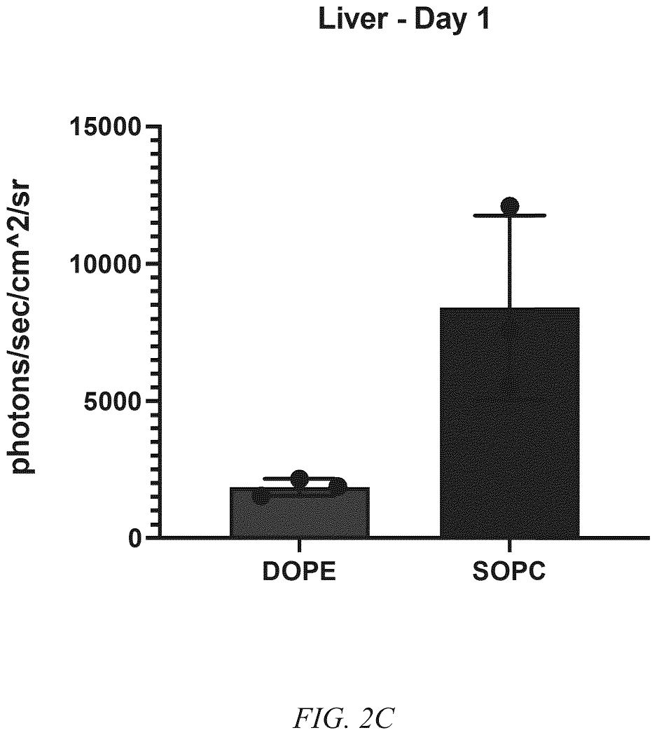

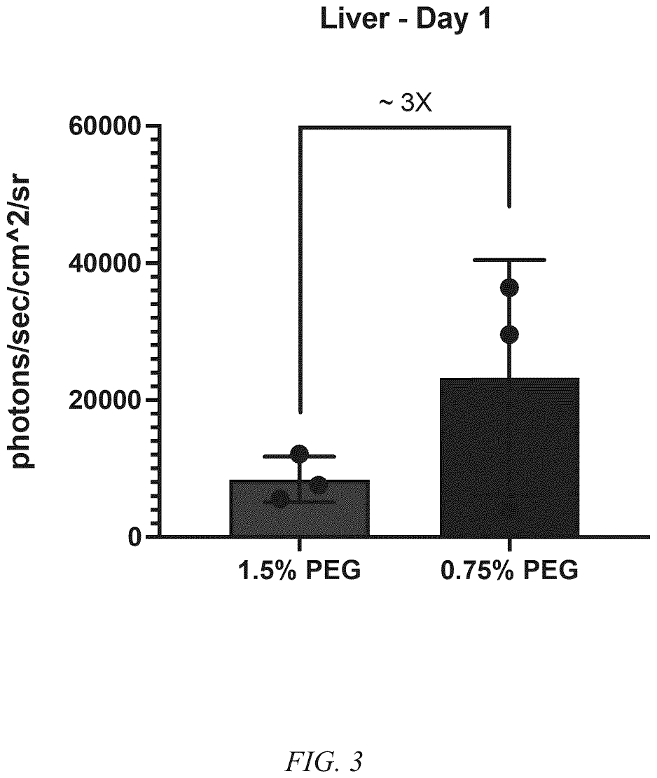

### DETAILED DESCRIPTION

The term “nucleic acid”, as used herein, generally refers to a polymer containing at least two deoxyribonucleotides or ribonucleotides in single- or double-stranded form and includes DNA and RNA. The DNA can be in the form of, for example, antisense molecules, plasmid DNA, pre-condensed DNA, nanoplasmid DNA, closed-end linear DNA, a PCR product, vectors (P1, PAC, BAC YAC, artificial chromosomes), expression cassettes, chimeric sequences, chromosomal DNA or derivatives and combinations of these groups. Nucleic acids include nucleic acids containing known nucleotide analogues or modified framework residues or linkages, which are synthetic, naturally occurring and non-naturally occurring, and which have binding properties similar to the reference nucleic acid. Examples of such analogues include, without limitation, phosphorothioates, phosphoramidates, methylphosphonates, chiral-methylphosphonates, 2′-O-methyl ribonucleotides, and peptide-nucleic acids (PNAs).

“Nucleotides” generally contain a sugar deoxyribose (DNA) or ribose (RNA), a base and a phosphate group. The nucleotides can be linked together through phosphate groups. “Bases” generally include purines and pyrimidines, which further include natural compounds of adenine, thymine, guanine, cytosine, uracil, inosine and natural analogues, and synthetic derivatives of purines and pyrimidines, which include, but are not limited to, modifications that place new groups reactives such as, but not limited to, amines, alcohols, thiols, carboxylates and alkyl halides.

The term “lipid” generally refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes (e.g., esters of acids with polyols); (2) “compound lipids,” which include phospholipids and glycolipids; and (3) “derived lipids” such as steroids. In some cases, lipids are esters of acids with polyols, glycerolipids (e.g., glycerides or acylglycerols), phospholipids, sphingolipids, sulfolipids, or glycolipids (e.g., glycero-glycolipids, phospho-glycolipids, sphingo-glycolypids, sulfo-glycolipids). Lipids that are esters with polyols can be esters of fatty acids. The fatty acid may be saturated or unsaturated. Examples of unsaturated fatty acids include, but are not limited to, myristoleic acid, palmitoleic acid sapienic acid, oleic acid, elaidic acid, vaccenic acid, linoleic acid, linoelaidic acid, alpha-linoelaidic acid arachidonic acid, eicosapentaenoic acid, erucic acid, docosahexanoic acid, or any cis/trans double-bond isomers thereof. In certain embodiments, the lipid is oleic acid. In certain embodiments, the lipid is an isomer of oleic acid (e.g., the double bond is in a different location along the aliphatic chain relative to oleic acid). In certain embodiments, the lipid is an analog of oleic acid (e.g., the aliphatic chain is 1-10 carbons longer or 1-10 carbons shorter than the aliphatic chain of oleic acid). Examples of saturated fatty acids include, but are not limited to, caprylic acid, capric acid, lauric acid, myristic acid, palmitic acid, stearic acid, arachidic acid, behenic acid, lignoceric acid, and cerotic acid. In certain embodiments, an oleic acid analog is a compound wherein the carboxylic acid moiety of oleic acid replaced by a different group.

The term “lipid particle” includes a lipid formulation that can be used to deliver an active agent or therapeutic agent, such as a nucleic acid (e.g., an interfering RNA), to a target site of interest (e.g., cell, tissue, organ, and the like). In preferred embodiments, the lipid particle of the invention is a nucleic acid-lipid particle, which is typically formed from a cationic lipid, a non-cationic lipid, and optionally a conjugated lipid that prevents aggregation of the particle. In other preferred embodiments, the active agent or therapeutic agent, such as a nucleic acid, may be encapsulated in the lipid portion of the particle, thereby protecting it from enzymatic degradation. In some cases, a “lipid particle” is a lipid nanoparticle (LNP). The lipid particles can be prepared by any suitable method, including but not limited to microfluidic assembly or extrusion. In some embodiments, for a lipid particle (e.g., LNP composition), a particle has a particular composition. In some embodiments, for a lipid particle (e.g., LNP composition), each particle has a particular composition. In some embodiments, for a lipid particle (e.g., LNP composition), at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9% of the particles have a particular composition.

The term “helper lipid” generally refers to a lipid capable of increasing the effectiveness of delivery of lipid-based particles such as cationic lipid-based particles to a target, preferably into a cell. The helper lipid can be neutral, positively charged, or negatively charged. Preferably, the helper lipid is neutral or negatively charged. In some cases the helper lipid comprises a phospholipid. In some cases the phospholipid bears fully saturated hydrocarbon chains. Examples for helper lipids include 1,2-distearoyl-sn-glycero-3-phosphocholine (DSPC), 1,2-dioleoyl-sn-glycero-3-phosphoethanolamine (DOPE), 1, 2-dioleoylene-sn-glycero-3-phosphocholine (DLPC), Dipalmitoylphosphatidylcholine (DPPC), 1,2-dimyristoyl-sn-glycero-3-phosphocholine (DMPC), 1,2-Dimyristoyl-sn-glycero-3-phosphoethanolamine (DMPE), 1,2-dioleoyl-sn-glycero-3-phosphocholine (DOPC), oleic acid, or any of the lipids described in WO2017099823A1 (which is incorporated by reference herein for all purposes). The helper lipid can be a phospholipid generally, or any of 1-palmitoyl-2-oleoyl-sn-glycero-3-phosphocholine (POPC),12-di-O-octadecenyl-5-glycero-3-phosphocholine (18:0 Diether PC),1-oleoyl-2-cholesterylhemisuccinoyl-5-glycero-3-phosphocholine (OChemsPC),1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3 phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), or sphingomyelin. In certain embodiments, the helper lipid comprises or is a fatty acid. The fatty acid may be saturated or unsaturated. Examples of unsaturated fatty acids include, but are not limited to, myristoleic acid, palmitoleic acid sapienic acid, oleic acid, elaidic acid, vaccenic acid, linoleic acid, linoelaidic acid, alpha-linoelaidic acid arachidonic acid, eicosapentaenoic acid, erucic acid, docosahexanoic acid, or any cis/trans double-bond isomers thereof, or fully or partially unsaturated derivatives thereof. In certain embodiments, the lipid is oleic acid. In certain embodiments, the lipid is an isomer of oleic acid (e.g., the double bond is in a different location along the aliphatic chain relative to oleic acid). In certain embodiments, the lipid is an analog of oleic acid (e.g., the aliphatic chain is 1-10 carbons longer or 1-10 carbons shorter than the aliphatic chain of oleic acid). Examples of saturated fatty acids include, but are not limited to, caprylic acid, capric acid, lauric acid, myristic acid, palmitic acid, stearic acid, arachidic acid, behenic acid, lignoceric acid, and cerotic acid. In certain embodiments, an oleic acid analog is a compound wherein the carboxylic acid moiety of oleic acid replaced by a different group. In certain embodiments, the helper lipid is a glycine derivative of a fatty acid (e.g., A-palitoylglycine or A-oleoglycine). In certain embodiments, the helper lipid is a glycerolipid e.g., monoglyceride, diglyceride, triglyceride). In certain embodiments, the helper lipid is a monoglyceride. In certain embodiments, the helper lipid is a diglyceride. In certain embodiments, the helper lipid is a triglyceride. In certain embodiments, the helper lipid comprises a sugar moiety (e.g., saccharide, disaccharide, polysaccharide).

As used herein, the term “neutral lipid” generally refers to a lipid that is neutrally charged at about physiological pH. Neutral lipids, when present in the lipid particle, can be any of a number of lipid species which exist either in an uncharged or neutral zwitterionic form at physiological pH. Such lipids include, for example diacylphosphatidylcholines, diacylphosphatidylethanolamines, ceramides, sphingomyelins, dihydrosphingomyelins, cephalins, and cerebrosides. The selection of neutral lipids for use in the particles described herein is generally guided by consideration of, e.g., particle size and stability of the particles in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In some embodiments, lipids containing saturated fatty acids with carbon chain lengths in the range of C14 to C22 are preferred. In some embodiments, lipids with mono or diunsaturated fatty acids with carbon chain lengths in the range of C14 to C22 are used. Additionally, lipids having mixtures of saturated and unsaturated fatty acid chains can be used.

The term “cationic lipid” generally refers to a lipid carrying a net positive charge at about physiological pH. Such cationic lipids include, but are not limited to DLin-MC3-DMA ((6Z,9Z,28Z,31Z)-Heptatriaconta-6,9,28,31-tetraen-19-yl 4-(dimethylamino)butanoate), DLin-KC2-DMA (2-(2,2-di((9Z,12Z)-octadeca-9,12-dien-1-yl)-1,3-dioxolan-4-yl)-N,N-dimethylethan-1-amine), DOTAP, SM102 (9-Heptadecanyl 8-{(2-hydroxyethyl)[6-oxo-6-(undecyloxy)hexyl]amino}octanoate), ALC0315 ((4-hydroxybutyl)azanediyl)bis(hexane-6,1-diyl) bis(2-hexyldecanoate), cKK-E12 (3,6-bis(4-(bis(2-hydroxydodecyl)amino)butyl)piperazine-2,5-dione), 3-(didodecylamino)-N1,N1,4-tridodecyl-1-piperazineethanamine (KL10), N1-[2-(didodecylamino)ethyl]N1,N4,N4-tridodecyl-14-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), 2,2-dilinoleyl-4-(2 dimethylaminoethyl)-[1,3]-dioxolane (DLin-KC2-DMA), 1,2-dioleyloxy-N,N-dimethylaminopropane (DODMA), 2-({8 [(3P)-cholest-5-en-3-yl oxy]octyl}oxy) N,N dimethyl-3-[(9Z,12Z)-octadeca-9,12-dien-1-yloxy]propan-1-amine (Octyl-CLinDMA), (2R)-2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S) 2-({8-[(3P)-cholest-5-en-3-yloxy]octyl}oxy)-N,N-dimethyl-3-[(9Z, 12Z)-octadeca-9, 12-dien-1-yloxy]propan-1-amine (Octyl-CLinDMA (2S)), (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16, 19-dien-8-amine, (13Z,16Z)—N,N-dimethyldocosa-13,16-dien-5-amine, (12Z,15Z)—N,N dimethylhenicosa-12,15-dien-4-amine, (14Z, 17Z)—N,N-dimethyltricosa-14,17-dien-6-amine, (15Z, 18Z)—N,N-dimethyltetracosa-15,18-dien-7-amine, (18Z,21 Z)—N,N-dimethylheptacosa-18,21-dien-10-amine, (15Z, 18Z)— N,N-dimethyltetracosa-15, 18-dien-5-amine, (14Z, 17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z, 19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien40-amine, 1-[(11Z, 14Z)-1 nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z, 15Z)—N,N-dimethyl-2-nonylhenicosa-12, 15-dien-1-amine, (13Z, 16Z)—N,N-dimethyl-3-nonyldocosa-13, 16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z, 12Z)-octadeca-9,12-dien-1-yloxy]-3-(octyloxy)propan-2-amine, S—N,N-dimethyl-1-[(9Z, 12Z)-octadeca-9, 12-dien-1-yloxy]-3-(octyloxy)propan-2-amine, 1-{2-[(9Z, 12Z)-octadeca-9, 12-dien-1-yloxy]-1-[(octyloxy)methyl]ethyl (pyrrollidine, (2S)—N,N-dimethyl-1-[(9Z, 12Z)-octadeca-9, 12-dien-1-yloxy]-3-[(5Z)-oct-5-en-1-yloxyjpropan-2-amine, 1-(2-[(9Z, 12Z)-octadeca-9, 12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z, 12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z, 12Z)-octadeca-6,9, 12-trien-1-yloxy]-3-(octyloxy)propan-2-amine, (2S)-1-[(11Z, 14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z, 14Z)-icosa-11,14-dien-1-yloxy]-N,N-dimethyl-3-(octyloxy)propan-2-amine, 1-[(13Z, 16Z)-docosa-13, 16-dien-1-yloxy]-N,N-dimethyl-3-(octyl oxy)propan-2-amine, (2S)—1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z, 12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1 S,2S)-2-{[(1R,2R)-2-pentylcyclopropyljmethyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine, 2-[3-(4-Chlorophenyl)-1-[2-(4-chlorophenyl)sulfanylphenyl]-3-oxopropyl]sulfanylacetic acid, 1,19-Bis(2-butyloctyl) 10-[[3-(dimethylamino)propyl](1-oxononyl)amino]nonadecanedioate, 2-(dodecyldisulfanyl)ethyl 3-[2-[2-[bis[3-[2-(dodecyldisulfanyl)ethoxy]-3-oxopropyl]amino]ethyl-methylamino]ethylamino]propanoate, 1-[2-[2-[bis(2-hydroxytetradecyl)amino]ethoxy]ethyl-[2-[4-[2-[2-[bis(2-hydroxytetradecyl)amino]ethoxy]ethyl]piperazin-1-yl]ethyl]amino]tetradecan-2-ol, [2-[3-(diethylamino)propoxycarbonyloxymethyl]-3-(4,4-dioctoxybutanoyloxy)propyl](9Z,12Z)-octadeca-9,12-dienoate, 9,12-Octadecadienoic acid (9Z,12Z)—, 3-[4,4-bis(octyloxy)-1-oxobutoxy]-2-[[4-(dimethylamino)-1-oxobutoxy]methyl]propyl ester (ACI), BP Lipid 103 (SMILES string O═C(OCCCCCCCCCCC)CCCCCN(CCO)CCCCCC(═O)OC(CCCCCCCC)CCCCCCCC), 16-Oxa-4,8,12-triazapentacosanoic acid, 8,24-dimethyl-4,12-bis[3-[(8-methylnonyl)oxy]-3-oxopropyl]-15-oxo-, 8-methylnonyl ester, 14-Oxa-17,18-dithia-4,7,10-triazatriacontanoic acid, 4,10-bis[3-[2-(dodecyldithio)ethoxy]-3-oxopropyl]-7-methyl-13-oxo-, 2-(dodecyldithio)ethyl ester (ACI), and 3,5,9-Trioxa-4-phosphaheptacosan-1-aminium, 4-ethoxy-N,N,N-trimethyl-10-oxo-7-[(1-oxooctadecyl)oxy]-, 4-oxide, chloride (1:1), (7R)-(ACI), or any combination thereof.

The cationic lipid can also be a lipid described in WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, WO2012040184, WO2011153120, WO2011149733, WO2011090965, WO2011043913, WO2011022460, WO2012061259, WO2012054365, WO2012044638 and WO2013116126, U.S. Pat. Nos. 7,893,302, 7,404,969, 8,283,333, and 8,466,122 and US Patent Publication No. US20100036115, US20120202871, US20130064894, US20130129785, US20130150625, US20130178541, and US20130225836; the contents of each of which are herein incorporated by reference in their entirety.

The term “conjugated lipid” generally refers to a conjugated lipid that inhibits aggregation of lipid particles. Such conjugated lipids can include lipids (e.g., “parent lipids”) conjugated to long-chain repetitive polymers (e.g., PEG), and include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes. The conjugated lipid can also be a PEG-lipid described in e.g., WO2020061284A1, which is incorporated by reference herein for all purposes. In some embodiments the conjugated lipid is a PEG-lipid and comprises a particular molecular weight of PEG (e.g., CH2CH2O monomer units). In some embodiments, the molecular weight of PEG is from about 250 to about 6000 Daltons. In some embodiments, the molecular weight of PEG is from about 500 to about 5000 Daltons. In some embodiments, the molecular weight of PEG is at least about 250, at least about 500, at least about 550, at least about 600, at least about 750, at least about 1000, at least about 1250, at least about 1500, at least about 1750, at least about 2000, at least about 2250, at least about 2500, at least about 2750, at least about 3000, at least about 3250, at least about 3500, at least about 3750, at least about 4000, at least about 4250, at least about 4500, at least about 4750, at least about 5000, at least about 5250, at least about 5500, at least about 5750, or at least about 6000 Daltons, or any range between these values. In some embodiments, the molecular weight of PEG is at most about 500, at most about 550, at most about 600, at most about 750, at most about 1000, at most about 1250, at most about 1500, at most about 1750, at most about 2000, at most about 2250, at most about 2500, at most about 2750, at most about 3000, at most about 3250, at most about 3500, at most about 3750, at most about 4000, at most about 4250, at most about 4500, at most about 4750, at most about 5000, at most about 5250, at most about 5500, at most about 5750, or at most about 6000 Daltons, or any range between these values. In some embodiments, the parent lipid comprises C8 Ceramide (e.g., N-octanoyl-D-erythro-sphingosine), C16 ceramide (e.g., N-palmitoyl-D-erythro-sphingosine), DOPE (1,2-dioleoyl-sn-glycero-3-phosphoethanolamine), SOPE (e.g., 1-stearoyl-2-oleoyl-sn-glycero-3-phosphoethanolamine), DSG (e.g., distearoyl-rac-glycerol), DSPE (e.g., 1,2-distearoyl-sn-glycero-3-phosphoethanolamine), DMPE (e.g., 1,2-dimyristoyl-sn-glycero-3-phosphoethanolamine), DPPE (e.g., 1,2-dipalmitoyl-sn-glycero-3-phosphoethanolamine), or cholesterol (e.g., Cholest-5-en-3β-ol), or any combination thereof. In some embodiments, the conjugated lipid comprises C8 PEG2000 Ceramide, C16 PEG2000 Ceramide, C8 PEG5000 Ceramide, C16 PEG5000 Ceramide, C8 PEG750 Ceramide, C16 PEG750 Ceramide, DOPE PEG550, DOPE PEG750, DOPE PEG1000, DOPE PEG2000, DOPE PEG5000, SOPE PEG550, SOPE PEG750, SOPE PEG1000, SOPE PEG2000, SOPE PEG5000, DSG PEG550, DSG PEG750, DSG PEG1000, DSG PEG2000, DSG PEG5000, DSPE PEG550, DSPE PEG750, DSPE PEG1000, DSPE PEG2000, DSPE PEG5000, Cholesterol PEG600, Cholesterol PEG1000, Cholesterol PEG2000, DMPE PEG 500, DMPE PEG 750, DMPE PEG 1000, DMPE PEG 2000, DPPE PEG 500, DPPE PEG 750, DPPE PEG 1000, or DPPE PEG 2000, or any combination thereof.

As used herein, the term “structural lipid” generally refers to sterols and also to lipids containing sterol moieties. Incorporation of structural lipids in the lipid nanoparticle may help mitigate aggregation of other lipids in the particle. Structural lipids can be, but are not limited to, a sterol, cholesterol, fecosterol, ergosterol, bassicasterol, tomatidine, tomatine, ursolic, alpha-tocopherol, cholesteryl acetate, cholesteryl oleate, 25-hydroxy cholesterol, 7-ketositosterol, 5.6-epoxy cholesterol, or any combination thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

The term “sterol” generally refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure. In some cases, a sterol can be cholesterol, fecosterol, sitosterol, ergosterol, campesterol, stigmasterol, brassicasterol, tomatidine, tomatine, ursolic acid, alpha-tocopherol.

As used herein, the term “reference composition” can generally refer to a composition not having one or more functional features specific to the context. For example, a reference composition for a composition exhibiting increased delivery to a tumor cell can be a composition as described herein not exhibiting said increased delivery.

As used herein, the term “Alkyl” generally refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises sixteen to eighteen carbon atoms (i.e., C₅-C₁₅ alkyl).In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more substituents such as those substituents described herein.

As used herein, the term “acyl” generally refers to an organic acid group wherein the —OH of the carboxyl group has been replaced with another substituent and has the general formula RC(═O)—, wherein R is an alkyl, alkenyl, alkynyl, aryl, carboxylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

In some embodiments, substituents may include any substituents described herein, for example: halogen, hydroxy, oxo (═O), thioxo (═S), cyano (—CN), nitro (—NO₂), imino (=N—H), oximo (=N—OH), hydrazino (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)OR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, substituents can include any substituents described herein, for example: halogen, hydroxy, oxo (=O), thioxo (=S), cyano (—CN), nitro (—NO₂), imino (=N—H), oximo (=N—OH), hydrazino (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, and alkynyl each of which may be optionally substituted by alkyl, alkenyl, alkynyl, halogen, hydroxy, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, substituents can include any substituents described herein, for example: halogen, haloalkyl, oxo (=O), hydroxy, thioxo (=S), cyano (—CN), nitro (—NO₂), imino (=N—H), oximo (=N—OH), hydrazino (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2), and —R^{b}—S(O)ₜN(R^{a})₂ (where t is 1 or 2); and alkenyl, alkynyl, aryl, aralkyl, aralkenyl, aralkynyl, cycloalkyl, cycloalkylalkyl, heterocycloalkyl, heterocycloalkylalkyl, heteroaryl, heteroarylalkyl, wherein the alkenyl, alkynyl, haloalkyl, haloalkenyl, haloalkynyl, aryl, aralkyl, aralkenyl, aralkynyl, cycloalkyl, cycloalkylalkyl, heterocycloalkyl, heterocycloalkylalkyl, heteroaryl, and heteroarylalkyl each of which may be optionally substituted by alkyl, alkenyl, alkynyl, halogen, haloalkyl, haloalkenyl, haloalkynyl, hydroxy, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, hydroxy, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, substituents can include any substituents described herein, for example: halogen, hydroxy, fluoroalkyl, oxo (=O), cyano (—CN), nitro (—NO₂), —R^{b}—OR, —R^{b}—N(R^{a})₂, —R^{b}—C(O)R^{a}, —R^{b}—C(O)OR^{a}, —R^{b}—C(O)N(R^{a})₂, and —R^{b}—N(R^{a})C(O)Ra; and alkyl, aryl, cycloalkyl, heterocycloalkyl, and heteroaryl, each of which may be optionally substituted by alkyl, alkenyl, alkynyl, halogen, haloalkyl, haloalkenyl, haloalkynyl, oxo (=O), hydroxy, thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R b-C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, substituents can include any substituents described herein, for example: alkyl, halo, fluoroalkyl, oxo (=O), hydroxy, cyano (—CN), —R^{b}—OR, —R^{b}—N(R^{a})₂, —R^{b}—C(O)R^{a}, and —R^{b}—C(O)OR^{a}, wherein the alkyl may be optionally substituted by alkenyl, alkynyl, halogen, hydroxy, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, hydroxy, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜRa (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some cases, any of the lipid nanoparticle (LNP) compositions described herein can be administered intravenously, subcutaneously, intraventricularly, intrathecally, intracerebroventricularly, transdermally, intramuscularly, orally, by inhalation, nasally, rectally, intratumorally, or proxi-tumorally to a subject. Proxi-tumorally may denote administration to the tissue within proximity of a tumor, or administration into a region that would be predicted to be accessible to the tumor via the lymphatic system (e.g., an adjoining lymph node). Intratumoral or proxi-tumoral approaches may involve the use of additional imaging techniques such as e.g., endoscopic ultrasonography (see e.g., Shirley et al. Gastroenterol Res Pract. 2013; 2013: 207129) or via a brochioscope (see e.g., Rojas-Solano et al. J Bronchology Interv Pulmonol. 2018 July; 25(3): 168-17). In some embodiments, the composition is administered into at least one of the cervical, epitrochlear, supraclavicular, cervical, axillary, mediastinal, supratrochlear, mesenteric, inguinal, femoral, or popliteal lymph nodes. In some cases, lymph-node based administration may serve as a method of centralized local delivery to a tissue region.

The term “subject” generally refers to human or non-human animals. In some cases, the subject is a human. In some cases the subject is an individual suspected of having a cancer (e.g., a liver, ovarian, pancreatic, breast, lung, smooth muscle, bladder, cervix, kidney, skin, prostate, brain, neuron, astrocyte, testicle, colorectal, lymphoid, or bone cancer). In some cases, the subject has a cancer (e.g., a liver, ovarian, pancreatic, breast, lung, smooth muscle, bladder, cervix, kidney, skin, prostate, brain, neuron, astrocyte, testicle, colorectal, lymphoid, or bone cancer). In some cases, the subject has previously undergone treatment for a cancer (e.g., a liver, ovarian, pancreatic, breast, lung, smooth muscle, bladder, cervix, kidney, skin, prostate, brain, neuron, astrocyte, testicle, colorectal, lymphoid, or bone cancer). In some cases, the subject has previously undergone treatment for a cancer (e.g., a liver, ovarian, pancreatic, breast, lung, smooth muscle, bladder, cervix, kidney, skin, prostate, brain, neuron, astrocyte, testicle, colorectal, lymphoid, or bone cancer) and is in remission from said cancer.

In some cases, any of the lipid nanoparticle (LNP) compositions described herein can be used to deliver a nucleic acid (e.g., DNA) to a diseased cell (e.g., such that said delivery is selective for said diseased cell). In some cases the diseased cell is a tumor cell, a cell characteristic of an autoimmune disease (e.g., a T-cell or lymphocyte with self-directed activity, or a normal cell damaged by autoimmunity), or a cell characteristic of a neurodegenerative disease (e.g., a cell bearing a toxic amyloid or proximal to a toxic amyloid). In some cases a tumor cell is a liver, ovarian, pancreatic, breast, lung, smooth muscle, bladder, cervix, kidney, skin, prostate, brain, neuron, astrocyte, testicle, colorectal, lymphoid, or bone tumor cell. In some cases the diseased cell can be within a subject.

The term “vector” generally refers to a nucleic acid sequence (e.g., DNA) capable of transferring other operably-linked heterologous or recombinant nucleic acid sequences to target cells. In some examples, a vector is a minicircle, plasmid, nanoplasmid, yeast artificial chromosome (YAC), bacterial artificial chromosome (BAC), cosmid, phagemid, bacteriophage genome, or linear covalently closed-ended DNA molecule (e.g., a doggybone DNA molecule). In some cases, a vector is a non-viral vector. In some cases a vector is a non-viral DNA vector. In some cases a vector is a circular DNA vector. In some cases a vector is a linear DNA vector.

The term “minicircle” as used herein generally refers to a small, double stranded circular DNA molecule that provides for persistent, high-level expression of a sequence of interest that is present on the vector, which sequence of interest may encode a polypeptide, an shRNA, an anti-sense RNA, an siRNA, and the like. The sequence of interest is operably linked to regulatory sequences present on the minicircle vector, said regulatory sequences controlling its expression. Such minicircle vectors are described, for example in published U.S. Patent Application US20040214329, herein specifically incorporated by reference.

As a different form of vector, the term “nanoplasmid” generally refers to a vector that may comprise minimized bacterial ColE1 or R6K origin of replication (which provides for such nanoplasmids to be replicable in a bacterial host strain), a bacterial RNA-based selectable marker, and a eukaryotic gene region. Such nanoplasmids can comprise a sequence having at least 90% sequence identity to the mini-R6K origin of SEQ ID NO: 1 and/or a sequence having at least 90% sequence identity to the RNA-OUT selectable marker of SEQ ID NO: 2 Further details of nanoplasmids are described e.g., in U.S. Pat. No. 9,737,620B2, which is incorporated by reference herein for the purposes of describing nanoplasmid sequence elements.

| SEQ ID NO: | Description | Sequence (DNA) |
|---|---|---|
| 1 | Mini R6K | GGCTTGTTGTCCACAACCGTTAAACCTTAAAAGCTTTAAAAG |
| | origin | CCTTATATATTCTTTTTTTTCTTATAAAACTTAAAACCTTAG |
| | (mini- | AGGCTATTTAAGTTGCTGATTTATATTAATTTTATTGTTCAA |
| | ori)#1 | ACATGAGAGCTTAGTACGTGAAACATGAGAGCTTAGTACGTT |
| | | AGCCATGAGAGCTTAGTACGTTAGCCATGAGGGTTTAGTTCG |
| | | TTAAACATGAGAGCTTAGTACGTTAAACATGAGAGCTTAGTA |
| | | CGTACTATCAACAGGTTGAACTGCTGATC |
| 2 | RNA-OUT | GTAGAATTGGTAAAGAGAGTCGTGTAAAATATCGAGTTCGCA |
| | SELECTABLE | CATCTTGTTGTCTGATTATTGATTTTTGGCGAAACCATTTGA |
| | MARKER | TCATATGACAAGATGTGTATCTACCTTAACTTAATGATTTTG |
| | #1 | ATAAAAATCATTA |

### Example Embodiments

In some aspects, the present disclosure provides for a composition comprising: a plurality of nucleic acid-lipid particles, wherein each particle in the plurality of particles comprises: (a) a nucleic acid; (b) a cationic lipid; (c) a helper lipid comprising at least one fatty acid chain bearing an acyl chain with an unsaturation (e.g., a single unsaturation at carbon-8); (d) a conjugated lipid that inhibits aggregation of particles; and (e) a structural lipid. In some embodiments, said nucleic acid is deoxyribonucleic acid (DNA). In some embodiments, said conjugated lipid is a polyethylene glycol conjugated lipid (PEG-lipid). In some embodiments, said PEG-lipid is PEG-conjugated myristoyl diglyceride (DMG). In some embodiments, said single unsaturation of said second fatty acid chain of said helper lipid is a carbon-8 unsaturation. In some embodiments, said first unsaturated fatty acid chain of said helper lipid is oleoyl and said second fatty acid chain of said helper lipid is stearoyl. In some embodiments, said lipid bearing said fatty acid chain with said carbon-8 unsaturation is a phospholipid. In some embodiments, said helper lipid bears two C16-C18 carbon chains. In some embodiments, said helper lipid comprises a cationic head group. In some embodiments, said helper lipid comprises a choline headgroup. In some embodiments, said helper lipid comprises SOPC, POPC, or DOPC. In some embodiments, said lipids and said nucleic acid are present at a ratio of about 20:1 in said composition. In some embodiments, said PEG-lipid comprises at least one fatty acid chain with an unsaturation (e.g., a single unsaturation). In some embodiments, said PEG-lipid comprises two fatty acid chains with an unsaturation (e.g., a single unsaturation). In some embodiments, said unsaturation (e.g., said single unsaturation) of said PEG-lipid occurs at carbon 8. In some embodiments, said PEG-lipid comprises PEG-conjugated 1,2-dioleoyl-sn-glycero-3-phosphoethanolamine (DOPE). In some embodiments, said PEG-lipid comprises PEG-conjugated 1-stearoyl-2-oleoyl-sn-glycero-3-phosphoethanolamine (SOPE). In some embodiments, said cationic lipid is present at a mole % of about at least 30% to about at least 85%. In some embodiments, said helper lipid is present at a mole % of about 8% to about 49.5%. In some embodiments, said PEG-lipid is present at a mole % of about 0.5% to about 8%. In some embodiments, said structural lipid comprises a sterol. In some embodiments, said sterol comprises cholesterol. In some embodiments, said cationic lipid comprises DLin-MC3, DLin-KC2, SM102, CKK12, ALC0315, or any combination thereof.

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a conjugated lipid that inhibits aggregation of particles, comprising at least one acyl chain (e.g., a fatty acyl chain) with an unsaturation (e.g., a single unsaturation); and (e) a sterol. In some embodiments, said conjugated lipid comprises a polyethylene glycol conjugated lipid (PEG-lipid). In some embodiments, said PEG-lipid comprises two acyl chains (e.g., two fatty acyl chains) each having an unsaturation (e.g., a single unsaturation). In some embodiments, said single unsaturation of said PEG-lipid occurs at carbon 8. In some embodiments, said PEG-lipid comprises PEG-conjugated 1,2-dioleoyl-sn-glycero-3-phosphoethanolamine (DOPE). In some embodiments, said PEG-lipid comprises PEG-conjugated 1-stearoyl-2-oleoyl-sn-glycero-3-phosphoethanolamine (SOPE). In some embodiments, said helper lipid comprises at least one acyl (e.g., fatty acyl) chain bearing an unsaturation (e.g., a single unsaturation). In some embodiments, said unsaturation of said second acyl (e.g., fatty acyl) chain of said helper lipid is a carbon-8 unsaturation. In some embodiments, said first unsaturated acyl chain of said helper lipid is oleoyl and said second acyl chain of said helper lipid is stearoyl. In some embodiments, said lipid comprising said acyl chain with said carbon-8 unsaturation is a phospholipid or a glycerolipid. In some embodiments, said helper lipid comprises two C16-C18 carbon chains. In some embodiments, said helper lipid comprises a cationic head group. In some embodiments, said helper lipid comprises a choline headgroup. In some embodiments, said helper lipid comprises SOPC, POPC, or DOPC. In some embodiments, said lipids and said nucleic acid are present at a ratio of about 20:1 in said composition. In some embodiments, said composition comprises a plurality of conjugated lipids that inhibit aggregation of particles, (i) a first conjugated lipid bearing at least one acyl (e.g., fatty acyl) chain with a carbon-8 unsaturation; and (ii) a second conjugated lipid bearing two saturated C18 acyl (e.g., fatty acyl) chains. In some embodiments, said first conjugated lipid is SOPE or DOPE. In some embodiments, said second conjugated lipid is PEG conjugated distearoyl-rac-glycerol (DSG).

In some aspects, the present disclosure provides for a composition comprising: a plurality of nucleic acid-lipid particles, wherein each particle in the plurality of particles comprises: (a) a nucleic acid; (b) a cationic lipid; (c) a helper lipid; (d) a conjugated lipid that inhibits aggregation of particles; and (e) a carbon-5 saturated cholesterol derivative. In some embodiments, said carbon-5 saturated cholesterol derivative is a carbon-24 alkyl-substituted cholesterol derivative. In some embodiments, said carbon-5 saturated cholesterol derivative comprises stigmastanol (5α-Stigmastan-3β-ol). In some embodiments, said carbon-5 saturated cholesterol derivative is according to:

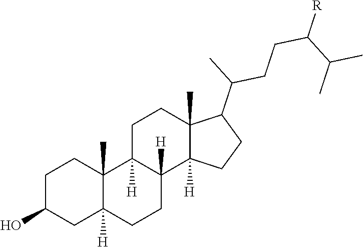

Wherein: R is selected from substituted or unsubstituted C₁-C₈ alkyl. In some embodiments, said helper lipid comprises at least one acyl (e.g., fatty acyl) chain bearing an unsaturation (e.g., a single unsaturation). In some embodiments, said PEG-lipid comprises at least one acyl chain with an unsaturation (e.g., a single unsaturation).

In some aspects, the present disclosure provides for a method of delivering DNA to a mammalian cell, comprising: contacting a mammalian cell with any of the compositions described herein. In some embodiments, said mammalian cell is a tumor cell.

In some aspects, the present disclosure provides for a composition comprising: a plurality of nucleic acid-lipid particles, wherein each particle in the plurality of particles comprises: (a) DNA; (b) a cationic lipid; (c) a helper lipid; (d) a conjugated lipid that inhibits aggregation of particles comprising about 3 mole % of the total lipid present in the particle; and (e) a sterol. In some embodiments, said conjugated lipid is a PEG-lipid. In some embodiments, said conjugated lipid is a glycerol derivative bearing two saturated C18 acyl (e.g., fatty acyl) chains. In some embodiments, said PEG-lipid comprises PEG-chain of about 2000 Daltons in molecular weight. In some embodiments, said conjugated lipid is DSG-PEG2000. In some embodiments, said helper lipid comprises at least one acyl (e.g., fatty acyl)chain bearing an unsaturation (e.g., a single unsaturation). In some embodiments, said unsaturation of said second acyl (e.g., fatty acyl)chain of said helper lipid is a carbon-8 unsaturation. In some embodiments, said first unsaturated acyl (e.g., fatty acyl)chain of said helper lipid is oleoyl and said second acyl (e.g., fatty acyl) chain of said helper lipid is stearoyl. In some embodiments, said lipid bearing said acyl (e.g., fatty acyl) chain with said carbon-8 unsaturation is a phospholipid. In some embodiments, said helper lipid bears two C16-C18 carbon chains. In some embodiments, said helper lipid comprises a cationic head group. In some embodiments, said helper lipid comprises a choline headgroup. In some embodiments, said helper lipid comprises SOPC, POPC, or DOPC. In some embodiments, said sterol comprises a carbon-5 saturated cholesterol derivative. In some embodiments, said carbon-5 saturated cholesterol derivative is a carbon-24 alkyl-substituted cholesterol derivative. In some embodiments, said carbon-5 saturated cholesterol derivative comprises stigmastanol (5α-Stigmastan-3β-ol). In some embodiments, said carbon-5 saturated cholesterol derivative is according to:

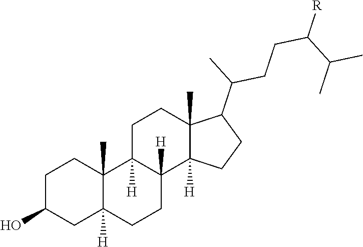

wherein: R is selected from substituted or unsubstituted C₁-C₈ alkyl.

In some cases, a cationic lipid in an LNP described herein may be present at a positive amount of not more than about 70 mol %, about 65 mol %, about 60 mol %, about 50 mol %, about 65 mol %, about 60 mol %, about 55 mol %, about 49 mol %, about 45 mol %, about 40 mol %, about 35 mol %, about 30 mol %, about 25 mol %, or about 20 mol % of the total lipid present in the particle, or any range between these values. In some cases, a cationic lipid in an LNP described herein may be present at an amount equal to or greater than about 20 mol %, about 25 mol %, about 30 mol %, about 35 mol %, about 40 mol %, about 45 mol %, about 49 mol %, about 50 mol %, about 55 mol %, about 60 mol %, about 65 mol %, or about 69 mol % of the total lipid present in the particle, or any range between these values. In some cases, a cationic lipid in an LNP described herein may be present at an amount of about 70 mol %, about 65 mol %, about 60 mol %, about 55 mol %, about 50 mol %, about 65 mol %, about 60 mol %, about 55 mol %, about 49 mol %, about 45 mol %, about 40 mol %, about 35 mol %, about 30 mol %, about 25 mol %, or about 20 mol % of the total lipid present in the particle, or any range between these values. In some cases, a cationic lipid in an LNP described herein may be present at an amount of about 70 mol % to about 20 mol %, about 65 mol % to about 20 mol %, about 60 mol % to about 20 mol %, about 50 mol % to about 20 mol %, about 65 mol % to about 20 mol %, about 60 mol % to about 20 mol %, about 55 mol % to about 20 mol %, about 49 mol % to about 20 mol %, about 45 mol % to about 20 mol %, about 40 mol % to about 20 mol %, about 35 mol % to about 20 mol %, about 30 mol % to about 20 mol %, or about 25 mol % to about 20 mol % of the total lipid present in the particle, or any range between these values. In some cases, a cationic lipid in an LNP described herein may be present at an amount from about 20 mol % to about 70 mol %, about 20 mol % to about 65 mol %, about 20 mol % to about 60 mol %, about 20 mol % to about 55 mol %, about 20 mol % to about 49 mol %, about 20 mol % to about 45 mol %, about 20 mol % to about 40 mol %, about 20 mol % to about 35 mol %, about 20 mol % to about 30 mol %, or about 20 mol % to about 25 mol % of the total lipid present in the particle. In some cases, a cationic lipid in an LNP described herein may be present at an amount from about 25 mol % to about 70 mol %, about 25 mol % to about 65 mol %, about 25 mol % to about 60 mol %, about 25 mol % to about 55 mol %, about 25 mol % to about 49 mol %, about 25 mol % to about 45 mol %, about 25 mol % to about 40 mol %, about 25 mol % to about 35 mol %, or about 25 mol % to about 30 mol % of the total lipid present in the particle. In some cases, a cationic lipid in an LNP described herein may be present at an amount from about 30 mol % to about 70 mol %, about 30 mol % to about 65 mol %, about 30 mol % to about 60 mol %, about 30 mol % to about 55 mol %, about 30 mol % to about 49 mol %, about 30 mol % to about 45 mol %, about 30 mol % to about 40 mol %, or about 30 mol % to about 35 mol %, of the total lipid present in the particle. In some cases, a cationic lipid in an LNP described herein may be present at an amount from about 40 mol % to about 70 mol %, about 40 mol % to about 65 mol %, about 40 mol % to about 60 mol %, about 40 mol % to about 55 mol %, about 40 mol % to about 49 mol %, or about 40 mol % to about 45 mol % of the total lipid present in the particle. In some cases, a cationic lipid in an LNP described herein may be present at an amount from about 45 mol % to about 70 mol %, about 45 mol % to about 65 mol %, about 45 mol % to about 60 mol %, about 45 mol % to about 55 mol %, or about 45 mol % to about 49 mol % of the total lipid present in the particle. In some cases, a cationic lipid in an LNP described herein may be present at an amount from about 49 mol % to about 70 mol %, about 49 mol % to about 65 mol %, about 49 mol % to about 60 mol %, or about 49 mol % to about 55 mol % of the total lipid present in the particle. In some cases, a cationic lipid in an LNP described herein may be present at an amount from about 55 mol % to about 70 mol %, about 55 mol % to about 65 mol %, or about 55 mol % to about 60 mol % of the total lipid present in the particle. In some cases, a cationic lipid in an LNP described herein may be present at an amount from about 60 mol % to about 70 mol %, about 60 mol % to about 65 mol % of the total lipid present in the particle. In some cases, a cationic lipid in an LNP described herein may be present at an amount from about 65 mol % to about 70 mol % of the total lipid present in the particle. In some cases, where the helper lipid, structural lipid, and conjugated lipid concentrations are defined, the remainder of the LNP formulation comprises the cationic lipid to make up 100 mol %.

In some cases, a helper lipid in an LNP described herein may be present at a positive amount of not more than about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 30 mol %, about 35 mol %, or about 40 mol % of the total lipid present in the particle, or any range between these values. In some cases, a helper lipid in an LNP described herein may be present at an amount equal to or greater than about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 30 mol %, about 35 mol %, or about 40 mol % of the total lipid present in the particle, or any range between these values. In some cases, a helper lipid in an LNP described herein may be present at an amount of about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 30 mol %, about 35 mol %, or about 40 mol % of the total lipid present in the particle, or any range between these values. In some cases, a helper lipid in an LNP described herein may be present at an amount of about 5 mol % to about 35 mol %. In some cases, a helper lipid in an LNP described herein may be present at an amount of about 5 mol % to about 6 mol %, about 5 mol % to about 7 mol %, about 5 mol % to about 8 mol %, about 5 mol % to about 9 mol %, about 5 mol % to about 10 mol %, about 5 mol % to about 11 mol %, about 5 mol % to about 12 mol %, about 5 mol % to about 13 mol %, about 5 mol % to about 14 mol %, about 5 mol % to about 15 mol %, about 5 mol % to about 16 mol %, about 5 mol % to about 17 mol %, about 5 mol % to about 18 mol %, about 5 mol % to about 19 mol %, about 5 mol % to about 20 mol %, about 5 mol % to about 21 mol %, about 5 mol % to about 22 mol %, about 5 mol % to about 23 mol %, about 5 mol % to about 24 mol %, about 5 mol % to about 25 mol %, about 5 mol % to about 30 mol %, about 5 mol % to about 35 mol %, or about 5 mol % to about 40 mol % of the total lipid present in the particle, or any range between these values. In some cases, a helper lipid in an LNP described herein may be present at an amount from 5 mol % to about 40 mol %, about 6 mol % to about 40 mol %, about 7 mol % to about 40 mol %, about 8 mol % to about 40 mol %, about 9 mol % to about 40 mol %, about 10 mol % to about 40 mol %, about 11 mol % to about 40 mol %, about 12 mol % to about 40 mol %, about 13 mol % to about 40 mol %, about 14 mol % to about 40 mol %, about 15 mol % to about 40 mol %, about 16 mol % to about 40 mol %, about 17 mol % to about 40 mol %, about 18 mol % to about 40 mol %, about 19 mol % to about 40 mol %, about 20 mol % to about 40 mol %, about 21 mol % to about 40 mol %, about 22 mol % to about 40 mol %, about 23 mol % to about 40 mol %, about 24 mol % to about 40 mol %, about 25 mol % to about 40 mol %, about 30 mol % to about 40 mol %, or about 35 mol % to about 40 mol % of the total lipid present in the particle. In some cases, a helper lipid in an LNP described herein may be present at an amount from 5 mol % to about 35 mol %, about 6 mol % to about 35 mol %, about 7 mol % to about 35 mol %, about 8 mol % to about 35 mol %, about 9 mol % to about 35 mol %, about 10 mol % to about 35 mol %, about 11 mol % to about 35 mol %, about 12 mol % to about 35 mol %, about 13 mol % to about 35 mol %, about 14 mol % to about 35 mol %, about 15 mol % to about 35 mol %, about 16 mol % to about 35 mol %, about 17 mol % to about 35 mol %, about 18 mol % to about 35 mol %, about 19 mol % to about 35 mol %, about 20 mol % to about 35 mol %, about 21 mol % to about 35 mol %, about 22 mol % to about 35 mol %, about 23 mol % to about 35 mol %, about 24 mol % to about 35 mol %, about 25 mol % to about 35 mol %, or about 30 mol % to about 35 mol of the total lipid present in the particle. In some cases, a helper lipid in an LNP described herein may be present at an amount from 5 mol % to about 30 mol %, about 6 mol % to about 30 mol %, about 7 mol % to about 30 mol %, about 8 mol % to about 30 mol %, about 9 mol % to about 30 mol %, about 10 mol % to about 30 mol %, about 11 mol % to about 30 mol %, about 12 mol % to about 30 mol %, about 13 mol % to about 30 mol %, about 14 mol % to about 30 mol %, about 15 mol % to about 30 mol %, about 16 mol % to about 30 mol %, about 17 mol % to about 30 mol %, about 18 mol % to about 30 mol %, about 19 mol % to about 30 mol %, about 20 mol % to about 30 mol %, about 21 mol % to about 30 mol %, about 22 mol % to about 30 mol %, about 23 mol % to about 30 mol %, about 24 mol % to about 30 mol %, or about 25 mol % to about 30 mol of the total lipid present in the particle. In some cases, a helper lipid in an LNP described herein may be present at an amount from 5 mol % to about 25 mol %, about 6 mol % to about 25 mol %, about 7 mol % to about 25 mol %, about 8 mol % to about 25 mol %, about 9 mol % to about 25 mol %, about 10 mol % to about 25 mol %, about 11 mol % to about 25 mol %, about 12 mol % to about 25 mol %, about 13 mol % to about 25 mol %, about 14 mol % to about 25 mol %, about 15 mol % to about 25 mol %, about 16 mol % to about 25 mol %, about 17 mol % to about 25 mol %, about 18 mol % to about 25 mol %, about 19 mol % to about 25 mol %, about 20 mol % to about 25 mol %, about 21 mol % to about 25 mol %, about 22 mol % to about 25 mol %, about 23 mol % to about 25 mol %, about 24 mol % to about 25 mol %, about 25 mol % to about 25 mol %, about 30 mol % to about 25 mol %, or about 35 mol % to about 25 mol % of the total lipid present in the particle. In some cases, a helper lipid in an LNP described herein may be present at an amount from 5 mol % to about 20 mol %, about 6 mol % to about 20 mol %, about 7 mol % to about 20 mol %, about 8 mol % to about 20 mol %, about 9 mol % to about 20 mol %, about 10 mol % to about 20 mol %, about 11 mol % to about 20 mol %, about 12 mol % to about 20 mol %, about 13 mol % to about 20 mol %, about 14 mol % to about 20 mol %, about 15 mol % to about 20 mol %, about 16 mol % to about 20 mol %, about 17 mol % to about 20 mol %, about 18 mol % to about 20 mol %, or about 19 mol % to about 20 mol % of the total lipid present in the particle. In some cases, a helper lipid in an LNP described herein may be present at an amount from 5 mol % to about 16 mol %, about 6 mol % to about 16 mol %, about 7 mol % to about 16 mol %, about 8 mol % to about 16 mol %, about 9 mol % to about 16 mol %, about 10 mol % to about 16 mol %, about 11 mol % to about 16 mol %, about 12 mol % to about 16 mol %, about 13 mol % to about 16 mol %, about 14 mol % to about 16 mol %, or about 15 mol % to about 16 mol % of the total lipid present in the particle. In some cases, a helper lipid in an LNP described herein may be present at an amount from 5 mol % to about 15 mol %, about 6 mol % to about 15 mol %, about 7 mol % to about 15 mol %, about 8 mol % to about 15 mol %, about 9 mol % to about 15 mol %, about 10 mol % to about 15 mol %, about 11 mol % to about 15 mol %, about 12 mol % to about 15 mol %, about 13 mol % to about 15 mol %, or about 14 mol % to about 15 mol of the total lipid present in the particle. In some cases, a helper lipid in an LNP described herein may be present at an amount from 5 mol % to about 14 mol %, about 6 mol % to about 14 mol %, about 7 mol % to about 14 mol %, about 8 mol % to about 14 mol %, about 9 mol % to about 14 mol %, about 10 mol % to about 14 mol %, about 11 mol % to about 14 mol %, about 12 mol % to about 14 mol %, or about 13 mol % to about 14 mol % of the total lipid present in the particle. In some cases, a helper lipid in an LNP described herein may be present at an amount from 5 mol % to about 13 mol %, about 6 mol % to about 13 mol %, about 7 mol % to about 13 mol %, about 8 mol % to about 13 mol %, about 9 mol % to about 13 mol %, about 10 mol % to about 13 mol %, about 11 mol % to about 13 mol %, or about 12 mol % to about 13 mol % of the total lipid present in the particle. In some cases, a helper lipid in an LNP described herein may be present at an amount from 5 mol % to about 12 mol %, about 6 mol % to about 12 mol %, about 7 mol % to about 12 mol %, about 8 mol % to about 12 mol %, about 9 mol % to about 12 mol %, about 10 mol % to about 12 mol %, or about 11 mol % to about 12 mol % of the total lipid present in the particle. In some cases, a helper lipid in an LNP described herein may be present at an amount from 5 mol % to about 11 mol %, about 6 mol % to about 11 mol %, about 7 mol % to about 11 mol %, about 8 mol % to about 11 mol %, about 9 mol % to about 11 mol %, or about 10 mol % to about 11 mol % of the total lipid present in the particle. In some cases, a helper lipid in an LNP described herein may be present at an amount from 5 mol % to about 10 mol %, about 6 mol % to about 10 mol %, about 7 mol % to about 10 mol %, about 8 mol % to about 10 mol %, or about 9 mol % to about 10 mol of the total lipid present in the particle. In some cases, a helper lipid in an LNP described herein may be present at an amount from 5 mol % to about 9 mol %, about 6 mol % to about 9 mol %, about 7 mol % to about 9 mol %, or about 8 mol % to about 9 mol % of the total lipid present in the particle. In some cases, a helper lipid in an LNP described herein may be present at an amount from 5 mol % to about 8 mol %, about 6 mol % to about 8 mol %, or about 7 mol % to about 8 mol of the total lipid present in the particle. In some cases, where the cationic lipid, structural, and conjugated lipid concentrations are defined, the remainder of the LNP formulation comprises the helper lipid to make up the total to 100 mol %.

In some cases, a structural lipid (e.g., a sterol) in an LNP described herein may be present at a positive amount of not more than about 15 mol %, 20 mol %, 25 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 60 mol %, 65 mol %, 70 mol %, or 75 mol % of the total lipid present in the particle, or any range between these values. In some cases, a structural lipid (e.g., a sterol) in an LNP described herein may be present at an amount of equal to or more than about 15 mol %, 20 mol %, 25 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 60 mol %, 65 mol %, 70 mol %, or 75 mol % of the total lipid present in the particle, or any range between these values. In some cases, a structural lipid (e.g., a sterol) in an LNP described herein may be present at an amount of about 15 mol %, 20 mol %, 25 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 60 mol %, 65 mol %, 70 mol %, or 75 mol % of the total lipid present in the particle, or any range between these values. In some cases a structural lipid in an LNP described herein can be present at an amount of about 20 mol % to about 70 mol % of the total lipid concentration in the particle. In some cases, a structural lipid (e.g., a sterol) in an LNP described herein may be present at an amount of about 15 mol % to about 20 mol %, about 15 mol % to 25 mol %, about 15 mol % to 30 mol %, about 15 mol % to 31 mol %, about 15 mol % to 32 mol %, about 15 mol % to 33 mol %, about 15 mol % to 34 mol %, about 15 mol % to 35 mol %, about 15 mol % to 36 mol %, about 15 mol % to 37 mol %, about 15 mol % to 38 mol %, about 15 mol % to 39 mol %, about 15 mol % to 40 mol %, about 15 mol % to 41 mol %, about 15 mol % to 42 mol %, about 15 mol % to 43 mol %, about 15 mol % to 44 mol %, about 15 mol % to 45 mol %, about 15 mol % to 46 mol %, about 15 mol % to 47 mol %, about 15 mol % to 48 mol %, about 15 mol % to 49 mol %, about 15 mol % to 50 mol %, about 15 mol % to 51 mol %, about 15 mol % to 52 mol %, about 15 mol % to 53 mol %, about 15 mol % to 54 mol %, about 15 mol % to 55 mol %, about 15 mol % to 60 mol %, about 15 mol % to 65 mol %, about 15 mol % to 70 mol %, or about 15 mol % to 75 mol % of the total lipid present in the particle, or any range between these values. In some cases, a structural lipid (e.g., a sterol) in an LNP described herein may be present at an amount of about 15 mol % to about 75 mol %, 20 mol % to about 75 mol %, 25 mol % to about 75 mol %, 30 mol % to about 75 mol %, 31 mol % to about 75 mol %, 32 mol % to about 75 mol %, 33 mol % to about 75 mol %, 34 mol % to about 75 mol %, 35 mol % to about 75 mol %, 36 mol % to about 75 mol %, 37 mol % to about 75 mol %, 38 mol % to about 75 mol %, 39 mol % to about 75 mol %, 40 mol % to about 75 mol %, 41 mol % to about 75 mol %, 42 mol % to about 75 mol %, 43 mol % to about 75 mol %, 44 mol % to about 75 mol %, 45 mol % to about 75 mol %, 46 mol % to about 75 mol %, 47 mol % to about 75 mol %, 48 mol % to about 75 mol %, 49 mol % to about 75 mol %, 50 mol % to about 75 mol %, 51 mol % to about 75 mol %, 52 mol % to about 75 mol %, 53 mol % to about 75 mol %, 54 mol % to about 75 mol %, 55 mol % to about 75 mol %, 60 mol % to about 75 mol %, 65 mol % to about 75 mol %, or 70 mol % to about 75 mol % of the total lipid present in the particle, or any range between these values. In some cases, a structural lipid (e.g., a sterol) in an LNP described herein may be present at an amount of about 15 mol % to about 70 mol %, 20 mol % to about 70 mol %, 25 mol % to about 70 mol %, 30 mol % to about 70 mol %, 31 mol % to about 70 mol %, 32 mol % to about 70 mol %, 33 mol % to about 70 mol %, 34 mol % to about 70 mol %, 35 mol % to about 70 mol %, 36 mol % to about 70 mol %, 37 mol % to about 70 mol %, 38 mol % to about 70 mol %, 39 mol % to about 70 mol %, 40 mol % to about 70 mol %, 41 mol % to about 70 mol %, 42 mol % to about 70 mol %, 43 mol % to about 70 mol %, 44 mol % to about 70 mol %, 45 mol % to about 70 mol %, 46 mol % to about 70 mol %, 47 mol % to about 70 mol %, 48 mol % to about 70 mol %, 49 mol % to about 70 mol %, 50 mol % to about 70 mol %, 51 mol % to about 70 mol %, 52 mol % to about 70 mol %, 53 mol % to about 70 mol %, 54 mol % to about 70 mol %, 55 mol % to about 70 mol %, 60 mol % to about 70 mol %, or 65 mol % to about 70 mol % or any range between these values. In some cases, a structural lipid (e.g., a sterol) in an LNP described herein may be present at an amount of about 15 mol % to about 65 mol %, 20 mol % to about 65 mol %, 25 mol % to about 65 mol %, 30 mol % to about 65 mol %, 31 mol % to about 65 mol %, 32 mol % to about 65 mol %, 33 mol % to about 65 mol %, 34 mol % to about 65 mol %, 35 mol % to about 65 mol %, 36 mol % to about 65 mol %, 37 mol % to about 65 mol %, 38 mol % to about 65 mol %, 39 mol % to about 65 mol %, 40 mol % to about 65 mol %, 41 mol % to about 65 mol %, 42 mol % to about 65 mol %, 43 mol % to about 65 mol %, 44 mol % to about 65 mol %, 45 mol % to about 65 mol %, 46 mol % to about 65 mol %, 47 mol % to about 65 mol %, 48 mol % to about 65 mol %, 49 mol % to about 65 mol %, 50 mol % to about 65 mol %, 51 mol % to about 65 mol %, 52 mol % to about 65 mol %, 53 mol % to about 65 mol %, 54 mol % to about 65 mol %, 55 mol % to about 65 mol %, 60 mol % to about 65 mol %,or any range between these values. In some cases, a structural lipid (e.g., a sterol) in an LNP described herein may be present at an amount of about 15 mol % to about 60 mol %, 20 mol % to about 60 mol %, 25 mol % to about 60 mol %, 30 mol % to about 60 mol %, 31 mol % to about 60 mol %, 32 mol % to about 60 mol %, 33 mol % to about 60 mol %, 34 mol % to about 60 mol %, 35 mol % to about 60 mol %, 36 mol % to about 60 mol %, 37 mol % to about 60 mol %, 38 mol % to about 60 mol %, 39 mol % to about 60 mol %, 40 mol % to about 60 mol %, 41 mol % to about 60 mol %, 42 mol % to about 60 mol %, 43 mol % to about 60 mol %, 44 mol % to about 60 mol %, 45 mol % to about 60 mol %, 46 mol % to about 60 mol %, 47 mol % to about 60 mol %, 48 mol % to about 60 mol %, 49 mol % to about 60 mol %, 50 mol % to about 60 mol %, 51 mol % to about 60 mol %, 52 mol % to about 60 mol %, 53 mol % to about 60 mol %, 54 mol % to about 60 mol %, 55 mol % to about 60 mol % or any range between these values. In some cases, a structural lipid (e.g., a sterol) in an LNP described herein may be present at an amount of about 15 mol % to about 55 mol %, 20 mol % to about 55 mol %, 25 mol % to about 55 mol %, 30 mol % to about 55 mol %, 31 mol % to about 55 mol %, 32 mol % to about 55 mol %, 33 mol % to about 55 mol %, 34 mol % to about 55 mol %, 35 mol % to about 55 mol %, 36 mol % to about 55 mol %, 37 mol % to about 55 mol %, 38 mol % to about 55 mol %, 39 mol % to about 55 mol %, 40 mol % to about 55 mol %, 41 mol % to about 55 mol %, 42 mol % to about 55 mol %, 43 mol % to about 55 mol %, 44 mol % to about 55 mol %, 45 mol % to about 55 mol %, 46 mol % to about 55 mol %, 47 mol % to about 55 mol %, 48 mol % to about 55 mol %, 49 mol % to about 55 mol %, 50 mol % to about 55 mol %, 51 mol % to about 55 mol %, 52 mol % to about 55 mol %, 53 mol % to about 55 mol %, or 54 mol % to about 55 mol or any range between these values. In some cases, a structural lipid (e.g., a sterol) in an LNP described herein may be present at an amount of about 15 mol % to about 50 mol %, 20 mol % to about 50 mol %, 25 mol % to about 50 mol %, 30 mol % to about 50 mol %, 31 mol % to about 50 mol %, 32 mol % to about 50 mol %, 33 mol % to about 50 mol %, 34 mol % to about 50 mol %, 35 mol % to about 50 mol %, 36 mol % to about 50 mol %, 37 mol % to about 50 mol %, 38 mol % to about 50 mol %, 39 mol % to about 50 mol %, 40 mol % to about 50 mol %, 41 mol % to about 50 mol %, 42 mol % to about 50 mol %, 43 mol % to about 50 mol %, 44 mol % to about 50 mol %, 45 mol % to about 50 mol %, 46 mol % to about 50 mol %, 47 mol % to about 50 mol %, 48 mol % to about 50 mol %, or 49 mol % to about 50 mol % or any range between these values. In some cases, a structural lipid (e.g., a sterol) in an LNP described herein may be present at an amount of about 15 mol % to about 45 mol %, 20 mol % to about 45 mol %, 25 mol % to about 45 mol %, 30 mol % to about 45 mol %, 31 mol % to about 45 mol %, 32 mol % to about 45 mol %, 33 mol % to about 45 mol %, 34 mol % to about 45 mol %, 35 mol % to about 45 mol %, 36 mol % to about 45 mol %, 37 mol % to about 45 mol %, 38 mol % to about 45 mol %, 39 mol % to about 45 mol %, 40 mol % to about 45 mol %, 41 mol % to about 45 mol %, 42 mol % to about 45 mol %, 43 mol % to about 45 mol %, or 44 mol % to about 45 mol % or any range between these values. In some cases, a structural lipid (e.g., a sterol) in an LNP described herein may be present at an amount of about 15 mol % to about 40 mol %, 20 mol % to about 40 mol %, 25 mol % to about 40 mol %, 30 mol % to about 40 mol %, 31 mol % to about 40 mol %, 32 mol % to about 40 mol %, 33 mol % to about 40 mol %, 34 mol % to about 40 mol %, 35 mol % to about 40 mol %, 36 mol % to about 40 mol %, 37 mol % to about 40 mol %, 38 mol % to about 40 mol %, or 39 mol % to about 40 mol % of the total lipid present in the particle, or any range between these values. In some cases, a structural lipid (e.g., a sterol) in an LNP described herein may be present at an amount of about 15 mol % to about 35 mol %, 20 mol % to about 35 mol %, 25 mol % to about 35 mol %, 30 mol % to about 35 mol %, 31 mol % to about 35 mol %, 32 mol % to about 35 mol %, 33 mol % to about 35 mol %, or 34 mol % to about 35 mol % of the total lipid present in the particle, or any range between these values. In some cases, a structural lipid (e.g., a sterol) in an LNP described herein may be present at an amount of about 15 mol % to about 30 mol %, 20 mol % to about 30 mol %, or 25 mol % to about 30 mol % of the total lipid present in the particle, or any range between these values. In some cases, where the cationic lipid, helper lipid, and conjugated lipid concentrations are defined, the remainder of the LNP formulation comprises the structural lipid to make up the total amount to 100 mol %.

In some cases, a conjugated lipid that prevents aggregation of particles (e.g., a PEG-lipid) in an LNP described herein may be present at a positive amount of not more than about 0.1 mol %, 0.5 mol %, 0.75 mol %, 1.0 mol %, 1.25 mol %, 1.5 mol %, 1.75 mol %, 2.0 mol %, 2.25 mol %, 2.5 mol %, 3.0 mol %, 3.25 mol %, 3.5 mol %, 3.75 mol %, 4.0 mol %, 4.25 mol %, 4.5 mol %, 4.75 mol %, 5.0 mol %, 5.25 mol %, 5.5 mol %, or 6.0 mol % of the total lipid present in the particle, or any range between these values. In some cases, a conjugated lipid that prevents aggregation of particles (e.g., a PEG-lipid) in an LNP described herein may be present an amount equal to or greater than about 0.1 mol %, 0.5 mol %, 0.75 mol %, 1.0 mol %, 1.25 mol %, 1.5 mol %, 1.75 mol %, 2.0 mol %, 2.25 mol %, 2.5 mol %, 3.0 mol %, 3.25 mol %, 3.5 mol %, 3.75 mol %, 4.0 mol %, 4.25 mol %, 4.5 mol %, 4.75 mol %, 5.0 mol %, 5.25 mol %, 5.5 mol %, or 6.0 mol % of the total lipid present in the particle, or any range between these values. In some cases, a conjugated lipid that prevents aggregation of particles (e.g., a PEG-lipid) in an LNP described herein may be present at an amount of about 0.1 mol %, 0.5 mol %, 0.75 mol %, 1.0 mol %, 1.25 mol %, 1.5 mol %, 1.75 mol %, 2.0 mol %, 2.25 mol %, 2.5 mol %, 3.0 mol %, 3.25 mol %, 3.5 mol %, 3.75 mol %, 4.0 mol %, 4.25 mol %, 4.5 mol %, 4.75 mol %, 5.0 mol %, 5.25 mol %, 5.5 mol %, or 6.0 mol % of the total lipid present in the particle, or any range between these values. In some cases, a conjugated lipid that prevents aggregation of particles (e.g., a PEG-lipid) in an LNP described herein may be present at an amount of about 0.1 mol % to about 6.0 mol %, 0.5 mol % to about 6.0 mol %, 0.75 mol % to about 6.0 mol %, 1.0 mol % to about 6.0 mol %, 1.25 mol % to about 6.0 mol %, 1.5 mol % to about 6.0 mol %, 1.75 mol % to about 6.0 mol %, 2.0 mol % to about 6.0 mol %, 2.25 mol % to about 6.0 mol %, 2.5 mol % to about 6.0 mol %, 3.0 mol % to about 6.0 mol %, 3.25 mol % to about 6.0 mol %, 3.5 mol % to about 6.0 mol %, 3.75 mol % to about 6.0 mol %, 4.0 mol % to about 6.0 mol %, 4.25 mol % to about 6.0 mol %, 4.5 mol % to about 6.0 mol %, 4.75 mol % to about 6.0 mol %, 5.0 mol % to about 6.0 mol %, 5.25 mol % to about 6.0 mol %, or 5.5 mol % to about 6.0 mol % of the total lipid present in the particle, or any range between these values. In some cases, a conjugated lipid that prevents aggregation of particles (e.g., a PEG-lipid) in an LNP described herein may be present at an amount of about 0.1 mol % to about 0.5 mol %, about 0.1 mol % to about 0.75 mol %, about 0.1 mol % to about 1.0 mol %, about 0.1 mol % to about 1.25 mol %, about 0.1 mol % to about 1.5 mol %, about 0.1 mol % to about 1.75 mol %, about 0.1 mol % to about 2.0 mol %, about 0.1 mol % to about 2.25 mol %, about 0.1 mol % to about 2.5 mol %, about 0.1 mol % to about 3.0 mol %, about 0.1 mol % to about 3.25 mol %, about 0.1 mol % to about 3.5 mol %, about 0.1 mol % to about 3.75 mol %, about 0.1 mol % to about 4.0 mol %, about 0.1 mol % to about 4.25 mol %, about 0.1 mol % to about 4.5 mol %, about 0.1 mol % to about 4.75 mol %, about 0.1 mol % to about 5.0 mol %, about 0.1 mol % to about 5.25 mol %, about 0.1 mol % to about 5.5 mol %, or about 0.1 mol % to about 6.0 mol % of the total lipid present in the particle, or any range between these values. In some cases, a conjugated lipid that prevents aggregation of particles (e.g., a PEG-lipid) in an LNP described herein may be present at an amount of about 0.75 mol % to about 5.0 mol %, 1.0 mol % to about 5.0 mol %, 1.25 mol % to about 5.0 mol %, 1.5 mol % to about 5.0 mol %, 1.75 mol % to about 5.0 mol %, 2.0 mol % to about 5.0 mol %, 2.25 mol % to about 5.0 mol %, 2.5 mol % to about 5.0 mol %, 3.0 mol % to about 5.0 mol %, 3.25 mol % to about 5.0 mol %, 3.5 mol % to about 5.0 mol %, 3.75 mol % to about 5.0 mol %, 4.0 mol % to about 5.0 mol %, 4.25 mol % to about 5.0 mol %, 4.5 mol % to about 5.0 mol %, 4.75 mol % to about 5.0 mol %, 5.0 mol % to about 5.0 mol %, 5.25 mol % to about 5.0 mol %, or 5.5 mol % to about 5.0 mol % of the total lipid present in the particle, or any range between these values. In some cases, a conjugated lipid that prevents aggregation of particles (e.g., a PEG-lipid) in an LNP described herein may be present at an amount of about 0.75 mol % to about 1.0 mol %, 0.75 mol % to 1.25 mol %, 0.75 mol % to 1.5 mol % 0.75 mol % to 1.75 mol %, 0.75 mol % to 2.0 mol %, 0.75 mol % to 2.25 mol %, 0.75 mol to 2.5 mol %, 0.75 mol %, to 3.0 mol %, 0.75 mol % to 3.25 mol %, 0.75 mol to 3.5 mol %, 0.75 mol % to 3.75 mol %, 0.75 mol % to 4.0 mol %, 0.75 mol % to 4.25 mol %, 0.75 mol % to 4.5 mol %, 0.75 mol % to 4.75 mol %, or 0.75 mol, to 5.0 mol of the total lipid present in the particle, or any range between these values.

In some aspects, a nucleic acid-delivering (e.g., DNA-delivering) LNP composition is according to any of the compositions described in Tables 1A, 1B, 1C, 1D, 1E, 1F, or 1G below.

TABLE 1A
Formulation of LNP compositions according
to the current disclosure (mol %).

| | EM40 | | | |
|---|---|---|---|---|
| | (aka | | | |
| | FRM055) | JetPEI | FRM119 | FRM060 |
| JetPEI, mole % | — | 100 | — | — |
| Cationic lipid DLin-MC3- | 50 | | 50 | 50 |
| DMA), mole % | | | | |
| Helper lipid SOPC, mole % | 10 | | 10 | 10 |
| Helper lipid DSPC, mole % | | | | |
| Structural lipid Cholesterol, | 39.25 | | 37 | 38.5 |
| mole % | | | | |
| Conjugated lipid DMG- | 0.75 | | — | 1.5 |
| PEG2000, mole % | | | | |
| Conjugated lipid DSG- | — | | 3 | |
| PEG2000, mole % | | | | |
| N/P ratio | 5.79 | 8 | 5.79 | 5.79 |
| Mass ratio | 20 | | 21.9 | 20.58 |
| (Total Lipids:DNA) | | | | |
| Particle size (nm) | 102 | ~150 | 82 | 83.4 |
| Zeta potential (mV) | 2.1 | ~22 | −2.3 | −6.7 |

TABLE 1B
Formulation of LNP compositions according
to the current disclosure (mol %).

| | FRM129 | FRM131 | FRM146 | FRM156 |
|---|---|---|---|---|
| Cationic lipid DLin- | 50 | 50 | 50 | 50 |
| MC3-DMA, mole % | | | | |
| Helper lipid SOPC, | 10 | 10 | 10 | 10 |
| mole % | | | | |
| Helper lipid DSPC, | | | | |
| mole % | | | | |
| Structural lipid | 37 | 38.5 | 37 | 37 |
| Cholesterol, mole % | | | | |
| Conjugated lipid DSG- | | | | 2 |
| PEG 2000, mole % | | | | |
| Conjugated lipid DSPE- | 3 | 1.5 | | |
| PEG1000, mole % | | | | |
| Conjugated lipid DOPE- | | | 3 | 1 |
| PEG1000, mole % | | | | |
| N/P ratio | 5.79 | 5.79 | 5.79 | 5.79 |
| Mass ratio | 20.9 | 20.17 | 20.95 | 21.52 |
| (Total Lipids:DNA) | | | | |
| Particle size (nm) | 79 | 107 | 83 | 80 |
| Zeta potential (mV) | −3.7 | −7.0 | −6.6 | −7.7 |

TABLE 1C
Formulation of LNP compositions according
to the current disclosure (mol %).

| | FRM237 | FRM238 | FRM239 |
|---|---|---|---|
| Cationic lipid DLin-MC3-DMA, | 50 | 50 | 50 |
| mole % | | | |
| Helper lipid SOPC, mole % | 10 | 10 | 10 |
| Helper lipid DSPC, mole % | | | |
| Structural lipid Cholesterol, mole % | 37 | 38.5 | 37 |
| Conjugated lipid DSG-PEG2000, | 1.5% | 2% | 2% |
| mole % | | | |
| Conjugated lipid C16 Ceramide- | 1.5% | 1% | |
| PEG750, mole % | | | |
| Conjugated lipid C8 Ceramide- | | | 1% |
| PEG750, mole % | | | |
| N/P ratio | 5.79 | 5.79 | 5.79 |
| Mass ratio | 21.15 | 21.37 | 21.33 |
| (Total Lipids:DNA) | | | |
| Particle size (nm) | 85 | 100 | 101 |

TABLE 1D
LNP Compositions According to Example 5 (mol %).

| | FRM001 | FRM182 |
|---|---|---|
| Cationic lipid DLin-MC3-DMA, mole % | 50 | 50 |
| Structural lipid Cholesterol, mole % | 38.5 | 37 |
| Helper lipid DSPC, mole % | 10 | — |
| Helper lipid SOPC, mole % | — | 10 |
| Conjugated lipid DMG-PEG2k, mole % | 1.5 | — |
| Conjugated lipid DSG-PEG2k, mole % | — | 1.5 |
| Conjugated lipid DOPE-PEG1k, mole % | — | 1.5 |

TABLE 1E
LNP Compositions According to the disclosure (mol %).

| | FRM214 | FRM225 | FRM156 |
|---|---|---|---|
| Cationic lipid | 45 | 45 | 50 |
| DLin-MC3- | | | |
| DMA, mole % | | | |
| Helper lipid | 0 | 0 | 10 |
| SOPC, mole % | | | |
| Helper lipid | 16 | 16 | 0 |
| DSPC, mole % | | | |
| Structural lipid | 36 | 18 | 0 |
| Stigmastanol, mole % | | | |
| Structural lipid | 0 | 18 | 37 |
| Cholesterol, mole % | | | |
| Conjugated lipid | 2 | 2 | 2 |
| DSG-PEG2000, | | | |
| mole % | | | |
| Conjugated lipid | 1 | 1 | 1 |
| DOPE-PEG1000, | | | |
| mole % | | | |
| N/P ratio | 5.21 | 5.79 | 5.79 |
| Mass ratio | 22.31 | 24.6 | 21.5 |
| (Total Lipids:DNA) | | | |
| Particle size (nm) | 82 | 99 | 80 |
| Zeta potential (mV) | −6.5 | −2.1 | −7.7 |

TABLE 1F
LNP compositions according to the disclosure (mol %).

| | FRM441 | FRM445 | FRM444 |
|---|---|---|---|
| Cationic lipid DLin-MC3- | 35 | 35 | 50 |
| DMA, mole % | | | |
| Helper lipid SOPC, mole % | 16 | 10 | 10 |
| Structural lipid Cholesterol, | 46 | 26 | 0 |
| mole % | | | |
| Structural lipid B-sitosterol, | 0 | 26 | 37 |
| mol % | | | |
| Conjugated lipid DOPE- | 1 | 1 | 1 |
| PEG1000, mole % | | | |
| Conjugated lipid DSG- | 2 | 2 | 2 |
| PEG2000, mole % | | | |
| N/P ratio | 5.79 | 5.79 | 5.79 |
| Particle size (nm) | 72 | 80 | 85 |
| Zeta potential (mV) | −4.4 | −4.3 | −2.5 |

TABLE 1G
LNP compositions according to the disclosure (mol %).

| | FRM119 | FRM165 | FRM156 | FRM177 | FRM237 | FRM362 |
|---|---|---|---|---|---|---|
| Cationic | 50 | 35 | 50 | 35 | 50 | 35 |
| lipid | | | | | | |
| DLin- | | | | | | |
| MC3- | | | | | | |
| DMA, | | | | | | |
| mole % | | | | | | |
| Helper | 10 | 10 | 10 | 10 | 10 | 10 |
| lipid | | | | | | |
| SOPC, | | | | | | |
| mole | | | | | | |
| Structural | 37 | 52 | 37 | 52 | 37 | 52 |
| lipid | | | | | | |
| Cho- | | | | | | |
| lesterol, | | | | | | |
| mole % | | | | | | |
| Con- | 0 | 0 | 0 | 0 | 1.5 | 1.5 |
| jugated | | | | | | |
| lipid C16 | | | | | | |
| ceramide | | | | | | |
| PEG750 | | | | | | |
| Con- | 0 | 0 | 1 | 1 | 0 | 0 |
| jugated | | | | | | |
| lipid | | | | | | |
| DOPE- | | | | | | |
| PEG1000, | | | | | | |
| mole % | | | | | | |
| Con- | 3 | 3 | 2 | 2 | 1.5 | 1.5 |
| jugated | | | | | | |
| lipid | | | | | | |
| DSG- | | | | | | |
| PEG2000, | | | | | | |
| mole % | | | | | | |
| N/P ratio | 5.79 | 5.79 | 5.79 | 5.79 | 5.79 | 5.79 |

### EXAMPLES

### Example 1.—Formulation of Lipid Nanoparticles

### Overview

Lipid nanoparticles were formulated by a microfluidic assembly process (using, for example, a Benchtop NanoAssemblr from Precision Nanosystems or other similar apparatus from other manufacturers including Unchained Labs) whereby lipids dissolved in ethanol were rapidly mixed with an aqueous stream containing DNA dissolved in an acidic buffer. The rapid dilution of the ethanol solvent resulted in formation of lipid nanoparticles. The nascent lipid nanoparticles were dialyzed against PBS overnight (with one buffer exchange 2-3 hours after dialysis has started) to remove ethanol and bring the solution pH to 7.4. The dialyzed formulation was then concentrated by ultrafiltration (100 kDa MWCO). Formulations were passed through a 0.22 mm filter in an aseptic environment using a biosafety cabinet for sterilization then diluted to the desired final concentration (typically 0.2-0.3 mg DNA/mL) using sterile PBS (1×, pH 7.4). The appropriate dilution was determined by measuring the DNA concentration of the formulation after sterile filtration using a fluorescence-based assay (with the commercially available DNA intercalating dye PicoGreen).

The particles were characterized by dynamic light scattering, electrophoretic light scattering, and the fluorescence based PicoGreen assay to verify the particle size, zeta potential, and DNA encapsulation efficiency and concentration, respectively.

### Detailed Procedure

Lipids received from the suppliers (see table 1 below) were dissolved into stock solutions in ethanol or, if not soluble in ethanol, in chloroform. Lipid mixes were prepared in ethanol and the aqueous solution of DNA/citrate buffer solution was made by diluting the 2 mg/mL DNA stock in 10 mM citrate buffer to a concentration of 0.133 mg/mL, followed by brief vortexing for homogenization. To mix the ethanol and aqueous solutions, a microfluidic cartridge (Precision Nanosystems; Part No. NIT0004) was used with a Precision Nanosystems Benchtop NanoAssemblr instrument, or other equivalent apparatus were used. The DNA/citrate buffer solution and lipid/ethanol solution were put into separate syringes and inserted into the corresponding slots in the cartridge and mixed at a flow rate ratio of 3:1 of aqueous: ethanol at a total flow rate of 12 mL/minute. The resulting formulation was immediately transferred to a pre-hydrated Slide-a-lyzer Dialysis Cassette in pH 7.4 PBS 1×. The dialysis solution was stirred for 2-3 hours, the PBS 1× replaced with fresh solution, and the formulation was allowed to dialyze overnight while stirring. The formulation was concentrated using an Amicon centrifugal filter (100 kDa MWCO).

TABLE 1A
Formulation of LNP compositions according
to the current disclosure.

| | EM40 | | | |
|---|---|---|---|---|
| | (aka | | | |
| | FRM055) | JetPEI | FRM119 | FRM060 |
| JetPEI, mole % | — | 100 | — | — |
| DLin-MC3-DMA, mole % | 50 | | 50 | 50 |
| SOPC, mole % | 10 | | 10 | 10 |
| DSPC, mole % | | | | |
| Cholesterol, mole % | 39.25 | | 37 | 38.5 |
| DMG-PEG2000, mole % | 0.75 | | — | 1.5 |
| DSG-PEG2000, mole % | — | | 3 | |
| N/P ratio | 5.79 | 8 | 5.79 | 5.79 |
| Mass ratio | 20 | | 21.9 | 20.58 |
| (Total Lipids:DNA) | | | | |
| Particle size (nm) | 102 | ~150 | 82 | 83.4 |
| Zeta potential (mV) | 2.1 | ~22 | −2.3 | −6.7 |

TABLE 1B
Formulation of LNP compositions according
to the current disclosure.

| | FRM129 | FRM131 | FRM146 | FRM156 |
|---|---|---|---|---|
| DLin-MC3-DMA, | 50 | 50 | 50 | 50 |
| mole % | | | | |
| SOPC, mole % | 10 | 10 | 10 | 10 |
| DSPC, mole % | | | | |
| Cholesterol, mole % | 37 | 38.5 | 37 | 37 |
| DSG-PEG2000, mole % | | | | 2 |
| DSPE-PEG1000, mole % | 3 | 1.5 | | |
| DOPE-PEG1000, mole % | | | 3 | 1 |
| N/P ratio | 5.79 | 5.79 | 5.79 | 5.79 |
| Mass ratio | 20.9 | 20.17 | 20.95 | 21.52 |
| (Total Lipids:DNA) | | | | |
| Particle size (nm) | 79 | 107 | 83 | 80 |
| Zeta potential (mV) | −3.7 | −7.0 | −6.6 | −7.7 |

TABLE 1C
Formulation of LNP compositions according
to the current disclosure.

| | FRM237 | FRM238 | FRM239 |
|---|---|---|---|
| DLin-MC3-DMA, mole % | 50 | 50 | 50 |
| SOPC, mole % | 10 | 10 | 10 |
| DSPC, mole % | | | |
| Cholesterol, mole % | 37 | 38.5 | 37 |
| DSG-PEG2000, mole % | 1.5% | 2% | 2% |
| C16 Ceramide-PEG750, mole % | 1.5% | 1% | |
| C8 Ceramide-PEG750, mole % | | | 1% |
| N/P ratio | 5.79 | 5.79 | 5.79 |
| Mass ratio | 21.15 | 21.37 | 21.33 |
| (Total Lipids:DNA) | | | |
| Particle size (nm) | 85 | 100 | 101 |

### Example 2.—Evaluation of LNP Performance in Cultured Cells (General Protocol)

### Nuclear Delivery Assay

The purpose of labeling the DNA was to determine the percentage of nuclei that had DNA successfully delivered to them using flow cytometry. The DNA is labelled using a commercial kit sold by Mirus Bio (Label iT kit; product #: MIR 7021).

The Label IT reagent has 3 important components to its chemical structure: a Cy5 dye which is fluorescent (Ex646 Em662 nm/orange), a linker with a quaternary amine (polarity/formal charge promotes association with the DNA strand) and an electrophilic group (2-chloro ethylamine). The electrophilic unit enables the DNA to be labelled without prior modification. Since the labeling is not specific, the labeling density is controlled by the amount of reagent added and the reaction time.

The protocol provided by Mirus with the kit was followed except for an alteration in the ratio of dye used per amount of DNA such that a lower labeling density was achieved (for 1.4 mg of DNA, 70 μL of the LabeliT reagent was used). Briefly, a 1 mg/mL solution of CAG-GFP nanoplasmid was mixed with 10.5 mL of ultrapure DNAse and RNase-free water and 1.4 mL of 10× Buffer A in a 50 mL falcon tube, which was wrapped in aluminum foil, and 70 μL of the LabeliT reagent was added to the tube. The reaction mixture was incubated at 37 C for 3 hours, followed by precipitation with and ice-chilled ethanol and salt solution. Centrifugation was used to pellet the DNA which was washed with 70% ethanol, air-dried, and resuspended in ultrapure water. The concentration was determined using a nanodrop instrument, and the final concentration was adjusted to 2 mg/mL. The sample was stored at −20° C. until use.

To determine the labeling efficiency of the Cy5 Label IT tag on the DNA, the Cy5 labeled DNA was diluted in TE buffer at different dilutions and the absorbance value of the DNA nucleotides (260 nm) and the Cy5 dye (lambda max excitation=646 nm) was measured using a nanodrop.

H1299-wt cells were cultured in RPMI containing 10% fetal bovine serum with 1% penicillin-streptomycin. Cells were plated before passage number 10.

H1299-wt cells were plated in a clear, flat-bottom 24-well plate with 50,000 cells per well in 50 μL of RPMI media with 1% fetal bovine serum (no penicillin-streptomycin). The cells were allowed to incubate for 48 hours to slow cell division prior to treatment. On the day of treatment, cells were dosed with 50-500 ng of DNA per well in 10 μL of formulation solution. The cells were allowed to incubate for an additional 24 hours prior to harvesting for flow cytometry.

The media was removed, and the cells were washed with 500 μL of 1×PBS before 0.25 mL of TrypLE was added in each well. The cells were put in the incubator for ~3-5 minutes to detach the cells. To each well, 0.75 mL of RPMI (10% fetal bovine serum) was added and the contents were transferred to a 2 mL deep well plate. The cells were pelleted by spinning the plate in a pre-chilled centrifuge (4 C) at 500 rcf for 5 minutes. The media was removed, the cells were resuspended in 500 μL of PBS, vortex, and put on ice and pelleted again by spinning the plate in a pre-chilled centrifuge (4 C) at 500 rcf for 5 minutes. The PBS was removed, and the cells were suspended in 220 μL of 1×PBS.

From this 220 μL sample, 145 μL was pipetted into a separate tube for isolation of cell nuclei and the remaining 75 μL to be used directly for analysis by flow cytometry.

### Cell Analysis-Nuclei

Isolation of the nuclei was done using the commercially available Nuclei EZ prep kit (product #: NUC10). The 145 μL sample of suspended cells (from section 4.2.3.) was pelleted by spinning it in a pre-chilled centrifuge (4 C) at 500 rcf for 5 minutes. The PBS was removed and 1 mL of nuclei EZ buffer was added. The sample was briefly vortexed, kept on ice for 5 minutes, and pelleted by spinning in a pre-chilled centrifuge (4° C.) at 500 rcf for 5 minutes. The buffer was then removed, the cells were resuspended in 500 μL of nuclei EZ buffer, vortexed briefly and kept on ice for 5 minutes. The nuclei were pelleted by spinning down in a centrifuge, the buffer was removed, and the cells were resuspended in 70 μL of 1×PBS buffer. The samples were then analyzed using flow cytometry.

The cells were analyzed using a MACSQuant flow cytometer. Channels were used to measure the signal from the Cy5 label on the DNA (nuclear uptake) and the GFP reporter protein (to check that the signal was low, confirming no cell contamination). FlowJo software was used to gate all the following populations: live cells—single cells—GFP positive or Cy5 positive cells (using the untreated population as the negative control.

### Cell Analysis-GFP Expression

H1299-wt cells were cultured in RPMI containing 10% fetal bovine serum with 1% penicillin-streptomycin. Cells were plated before passage number 10.

H1299-wt cells were plated in a clear, flat-bottom 48-well plate with 25,000 cells per well in 0.25 μL of RPMI media with 1% fetal bovine serum (no penicillin-streptomycin). The cells were allowed to incubate for 48 hours to slow cell division prior to treatment. On the day of treatment, cells were dosed with 25-150 ng of nanoplasmid DNA vector encoding GFP per well in 10 μL of formulation solution. The cells were allowed to incubate for an additional 24 hours prior to harvesting for flow cytometry.

The media was removed, and the cells were washed with 500 μL of 1×PBS before 0.25 mL of TrypLE was added in each well. The cells were put in the incubator for ~3-5 minutes to lift the cells. To each well, 0.75 mL of RPMI (10% fetal bovine serum) was added and the contents were transferred to a 2 mL deep well plate. The cells were pelleted by spinning the plate in a pre-chilled centrifuge (4 C) at 500 rcf for 5 minutes. The media was removed, the cells were resuspended in 500 PL of PBS, vortex, and put on ice and pelleted again by spinning the plate in a pre-chilled centrifuge (4 C) at 500 rcf for 5 minutes. The PBS was removed, and the cells were suspended in 80 PL of 1×PBS and analyzed using flow cytometry.

The cells were analyzed using a MACSQuant flow cytometer. Channels were used to measure the signal from the GFP reporter protein (transfection). FlowJo software was used to gate all the following populations: intact nuclei->single nuclei->Cy5 positive nuclei (using the untreated population as the negative control).

### Results

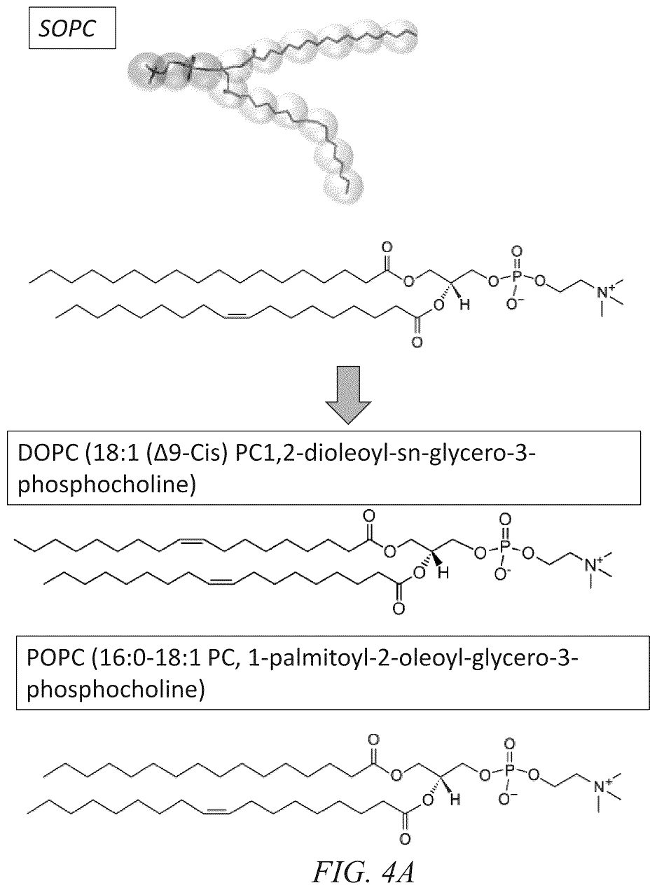

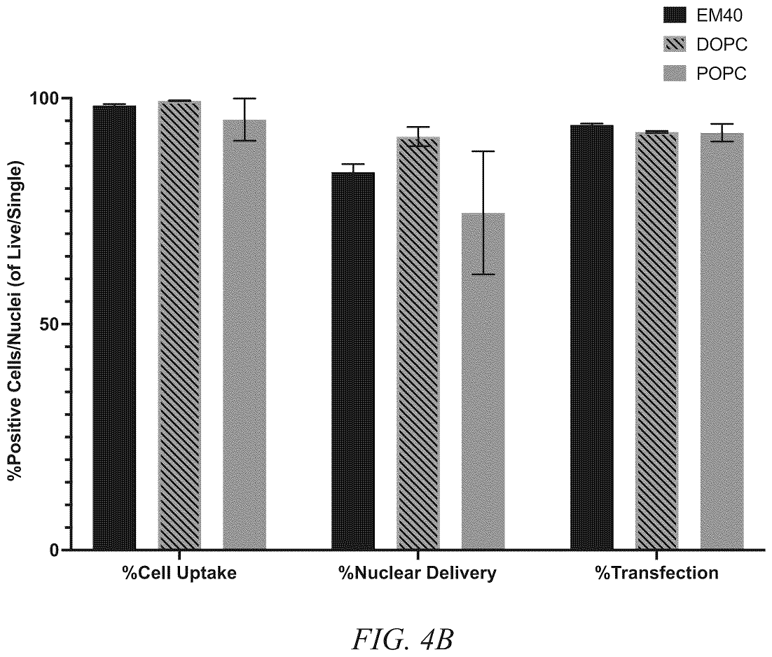

FIGS. 4A and 4B demonstrate that inclusion of other helper phospholipids bearing a fatty acid chain with a carbon-8 unsaturation (DOPC, POPC) also enhance DNA delivery when included in an LNP formulation. FIG. 4A compares the chemical structures of DOPC (18:1 (A9-Cis) PC or 1,2-dioleoyl-sn-glycero-3-phosphocholine) and POPC (16:0-18:1 PC or 1-palmitoyl-2-oleoyl-glycero-3-phosphocholine). FIG. 4B shows a bar graph on an in vitro experiment where DNA vectors were formulated into LNPs with 50 mole % cationic lipid (e.g., Dlin-MC3-DMA), 39.25 mole % cholesterol, 0.75 mole % PEG lipid (e.g., DMG-PEG) and either 10% SOPC (“EM-40”), 10% DOPC (“DOPC”), or 10% POPC (“POPC) and exposed to a cultured mammalian cell line (H129). Shown are percentage of cells positive of those live for cells that took up the DNA (“% Cell Uptake”), took up the DNA into their nucleus (“% Nuclear Delivery”), or successfully expressed the vector DNA-encoded reporter protein (“% Transfection”). For the “% Cell Uptake” and “% Nuclear Delivery” measures, DNA vector labeled with Cy5 was used in the LNPs, and uptake into either the cell overall or the nucleus specifically was performed 24 hours after introduction of the LNP by FACS for the Cy5 signal (nuclei measurements were performed on nuclei isolated from cells). For the “% Biomarker Expression” measurement, DNA vector encoding GFP was used in the LNPs, and the cells were assessed for the GFP signal by FACS. Both LNPs formulated with DOPC and POPC showed similar performance in all three measures to those formulated with SOPC.

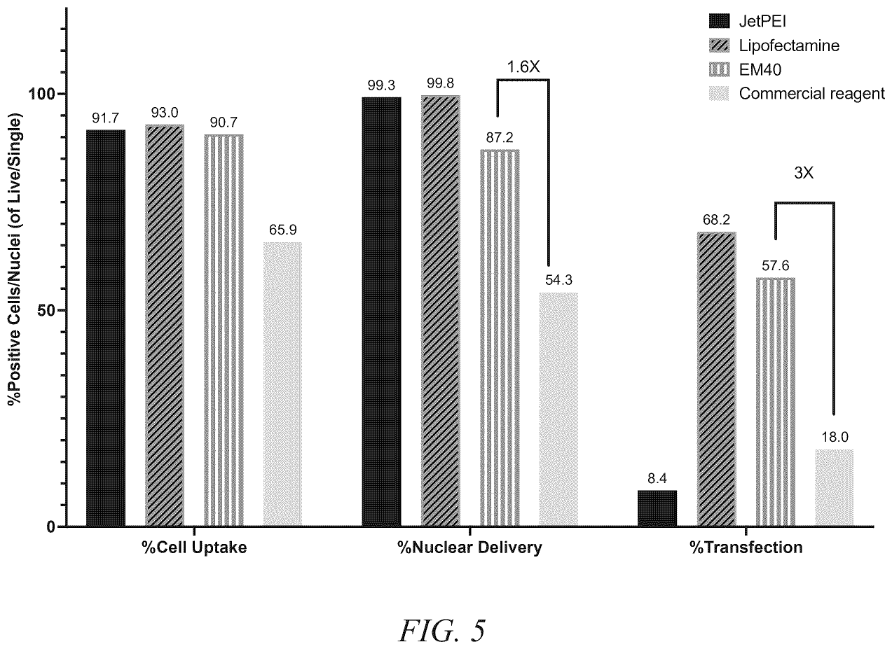

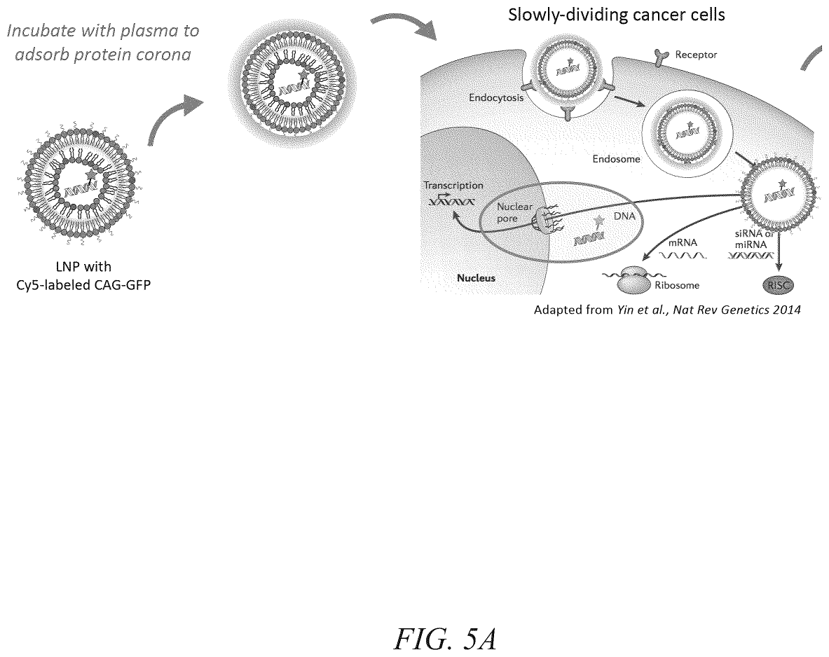

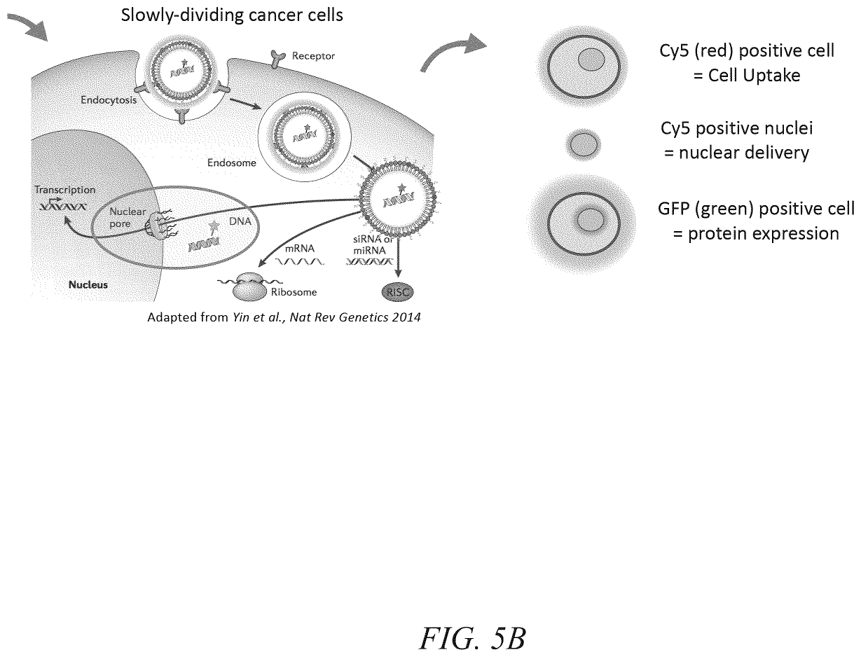

FIG. 5 demonstrates that LNP formulations using helper phospholipids bearing an acyl (e.g., fatty acyl) chain with a carbon-8 unsaturation and reduced PEG lipid (e.g., “EM40”) show superior delivery to cultured eukaryotic cells compared to a commercial LNP nucleic acid delivery formulation. Shown is a bar graph of a similar experiment to FIG. 4B where fluorescent protein encoding DNA vector was formulated into JetPEI according to manufacturer's instructions (“JetPEI”), lipofectamine according to manufacturer's instructions (“Lipofectamine”), LNPs with 50 mole % cationic lipid (e.g., Dlin-MC3-DMA), 39.25 mole % cholesterol, 0.75 mole % PEG lipid (e.g., DMG-PEG), and 10% SOPC (“EM40), or LNPs with 50 mole % cationic lipid (e.g., Dlin-MC3-DMA), 39.25 mole % cholesterol, 1.5 mole % PEG lipid (e.g., DMG-PEG), and 10% SOPC (“EM40”), or LNPs with 50 mole % cationic lipid (e.g., Dlin-MC3-DMA), 39.25 mole % cholesterol, 0.75 mole % PEG lipid (e.g., DMG-PEG), and 10% SOPC (“EM40”), or LNPs with 50 mole % cationic lipid (e.g., Dlin-MC3-DMA), 38.5 mole % cholesterol, 1.5 mole % PEG lipid (e.g., DMG-PEG), and 10% DSPC (“Commercial formulation”). Shown are percentage of cells positive of those live for cells that took up the DNA (“% Cell Uptake”), took up the DNA into their nucleus (“% Nuclear Delivery”), or successfully expressed the vector DNA-encoded reporter protein (“% Transfection”). “% Cell Uptake”, “% Nuclear Delivery”, and “% Transfection” was assessed as in FIG. 4B. Notably, the improved formulation (“EM40”) shows better performance for transfection versus a commercial formulation or JetPEI.

### Example 3.—LNP Testing In Vivo in Mouse Models

### Tolerability Testing in Naïve Mice

Immunocompetent Balb/c mice (stock 000651, Jackson Laboratory) were used at 7-10 weeks of age at dosing. Formulated nanoparticles with firefly luciferase-encoding vector (FLuc) driven by a constitutive CAG promoter were administered intravenously (IV, tail vein) in mice at a dose of 0.7-1.4 mg/kg in a volume of 10 mL/kg. Formulations tolerability was initially assessed at 1.4 mg/kg (e.g., half of the maximum tolerated dose, MTD from EM40-formulated DNA). Dose escalation was used to further determine MTD.

Body weight and clinical observations were monitored as indicators of tolerability. Body weight was measured prior to dosing (e.g., baseline) as well as 24 and 48 h post dosing. BW change was calculated as percent difference from baseline as: 100×(Value-Baseline)/(Baseline).

Clinical observations were recorded based on 5 categories. For each category, a score was attributed to the animal with 1 representing a healthy mouse and 5 the most severe observation within the category. A cumulative score was then calculated.

| | | Coat | Eyelid | | |
|---|---|---|---|---|---|
| category | Activity | condition | ptosis | Posture | Respiration |
| Score = 1 | Normal | Normal | Normal | Normal | Normal |
| Score = 3 | hypoactive | Slightly | Eyelid | Slightly | Slow labored |
| | | unkept | half shut | hunched | breathing |
| Score = 5 | no activity | Rough fur | Eyelid | Highly | Gasping for |
| | but | | fully | hunched | air |
| | reactive to | | closed | with | |
| | stimulation | | | inverted | |
| | | | | flanks | |

Normal healthy mice (i.e. PBS control group) present a cumulative score of 5. A maximum cumulative score was 25 (a top score of 5 across 5 categories) in theory but humane endpoint was set at 15 based on experience. An animal found dead or in moribund state is given a cumulative score of 15. Any animal reaching a cumulative clinical observation score of 15 or above (i.e. severe observation in at least 3 categories) was humanely euthanized. Experience has indicated that cumulative clinical observation score positively correlated with BW loss from baseline (i.e. the higher the cumulative score, the worse the BW loss; the data not shown).

To monitor vector expression, 24 h and 48 h later, in vivo bioluminescence imaging (BLI) was conducted as described below. Ex vivo organ BLI was conducted at 48 h post-dosing. In addition to expression, we routinely investigated peripheral blood exposure of formulated nanoparticles. To do so, blood samples were collected at 1h, 24 h and/or 48 h post-dosing into K2 EDTA tubes. After processing, plasma samples were stored at −80° C. awaiting qPCR analysis.

### Xenograft Model Establishment

H1299 cells obtained from ATCC were cultured in vitro in RPMI 1640 (ATCC: 30-2001) with 10% Fetal Bovine Serum (FBS, VWR: 89510-188) without Pen/Strep and cell banks cryopreserved at 2 e6 cells per cryovial. Cell vials from working cell banks were thawed for in vivo studies and cells were passaged when confluency was 80-90%. On the day of in vivo inoculation, cells were harvested by PBS (Corning: 21-040-CV) wash followed by trypsinization (TrypLE Express, Fisher Scientific: 50-591-419) and incubation at 37° C. for 5-10 minutes. TrypLE was quenched with complete culture media (RPMI 1640+10% FBS). The resulting cell suspension was centrifuged at 300×g for 5 min at Room Temperature. The cell pellet was resuspended in complete culture media, and counted on the Nexcelom Cell Counter (Nexcelom Biosciences, Spectrum Cell Counter, with Spectrum 5 Software) with either AOPI (Nexcelom, CS2-0106-25 mL) or Trypan Blue (Express Biotech, XV007CC). After a final PBS wash, cells were resuspended in PBS at 10×10⁶ H1299 cells per mL and kept on ice until inoculation. Pre-inoculation viability was consistently between 90-95%. Cell inoculation was conducted within 2 h of resuspension and viability drop was less than 15% post-inoculation.

NOD.Cg-Prkdc scid Il2rg tm1Wjl/SzJ mice (NSG, stock 005557, Jackson Laboratory) were used at 7-10 weeks of age at inoculation. The right flank of the mice (inoculation site) was shaved 1-3 days prior to inoculation. On the day of in vivo inoculation, 100 μL of cells suspension (e.g., 1×10⁶ H1299 cells per inoculation site) was injected subcutaneously under isoflurane (2-4%) anesthesia. Tumor burden was monitored weekly by caliper measurement. Animal welfare was monitored by weekly body weight and clinical observations upon finding. Once tumor volume (TV) reached 200-400 mm³, mice were randomized in treatment groups such that average TV was similar between groups. Animals' related data was recorded using Overwatch Research software (Benchling).

### Bioluminescence Imaging

To assess protein expression in vivo by LNP formulations, formulated nanoparticles with firefly luciferase-encoding vector (FLuc) driven by a constitutive CAG promoter were administered intravenously (IV, tail vein) in mice at a dose of 0.7-1.4 mg/kg in a volume of 10 mL/kg. 48 h later, animals were injected intraperitoneally (IP) with the FLuc substrate luciferin at 150 mg/kg in a volume of 10 mL/kg. The 24-48 h imaging timepoint was chosen because it corresponds to the peak of FLuc expression as previously assessed (data not shown). Mice were then anesthetized with 2-4% isoflurane and placed in an AMI HT system (Spectral Instruments Imaging) for bioluminescence imaging (BLI). Whole body imaging was initiated 12 min after IP substrate injection. At the terminal timepoint, immediately after whole body imaging, mice were euthanized and ex vivo tissue imaged.

The BLI signal in each region of interest (ROIs) drawn around individual whole body or tissue were quantified using the Aura software (Spectral Instruments Imaging). BLI signal intensity and location provide direct insights into the abundance and spatial distribution of FLuc in the imaged animal or tissue, thus indicating expression strength and specificity as well as delivery biodistribution.

IV-dosed PBS mice were also imaged to determine bioluminescent background signal. Individual BLI signal from the NP-dosed animals was corrected for the averaged background signal using the following formula: (corrected BLI)=(NP dosed BLI)/(average PBS dosed BLI).

Tumor signal was compared to other tissue signal by calculating a signal-to-noise (SNR) ratio for each animal as follows: SNR=(tumor corrected BLI)/(tissue corrected BLI).

### Pharmacokinetic Analysis

Pharmacokinetic analyses were performed on plasma and tissues harvested from animals after terminal euthanasia. Plasma was harvested by cardiac puncture into K2 EDTA tubes by standard procedures. Tissues (tumor, liver, lung, heart, spleen) were isolated from animals by necropsy and immediately flash frozen on dry ice. Both sets of samples were stored at −80° C. pending analysis. For vector DNA copy analysis, total DNA was first extracted from plasma or tissue using the appropriate QIAAMP DNA extraction kits (Qiagen). DNA was eluted into ultrapure water and quantified using a Qubit™ dsDNA assay. Equal quantities/volumes of DNA were then loaded into a 10 μL reaction in a 384-well plate qPCR instrument (ThermoFisher®) and a primer/probe set specific for the R6K sequence in nanoplasmid DNA. Serial dilutions of nanoplasmid DNA were used as to create a standard curve to enable absolute quantification. DNA copy numbers were then converted to copies per μL plasma or copy per cell, assuming 6 pg genomic DNA per cell.

### Results

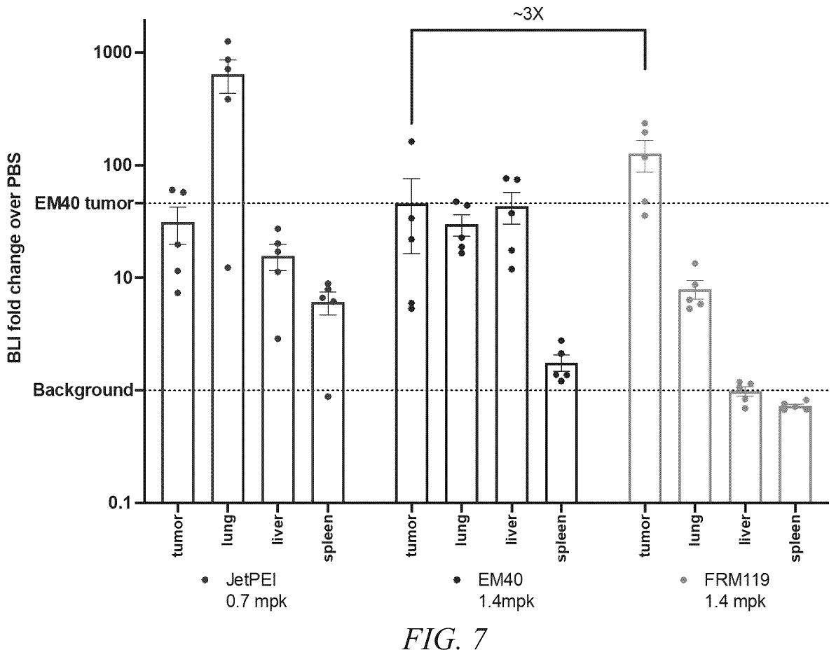

FIG. 7 demonstrates that LNP formulations described herein (e.g., “EM40”, “FRM119”) produce similar or better in vivo delivery tumors of a mouse H1299 xenograft model to JetPEI® (“JetPEI”). Shown are graphs of BLI imaging results on mice injected with LNP compositions of DNA vectors driving a BLI reporter. The graphs additionally demonstrate that further surface modification of LNP formulations to incorporate an increased concentration (e.g., 3 mole %) of a PEG-lipid comprising two saturated acyl chains (e.g., two saturated C18 acyl chains, “FRM 119”) improves delivery to tumor and lowers delivery to other tissues such as lung, liver, and spleen.

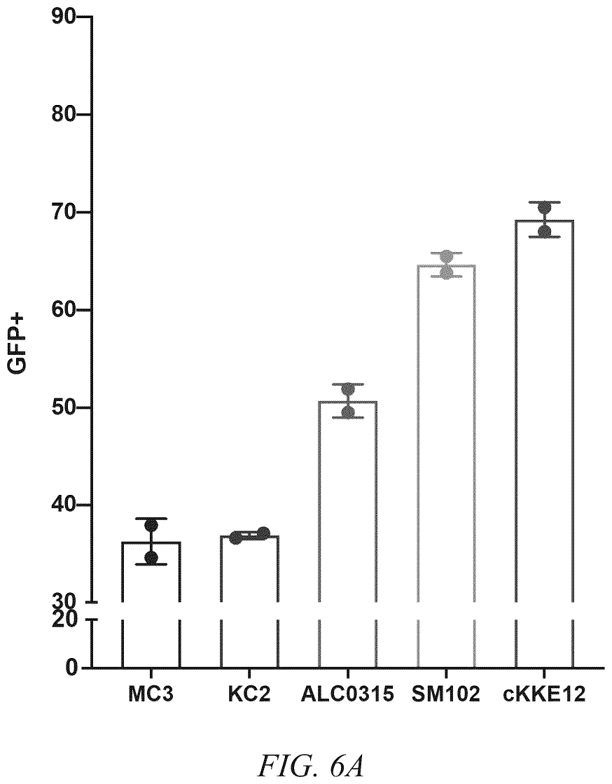

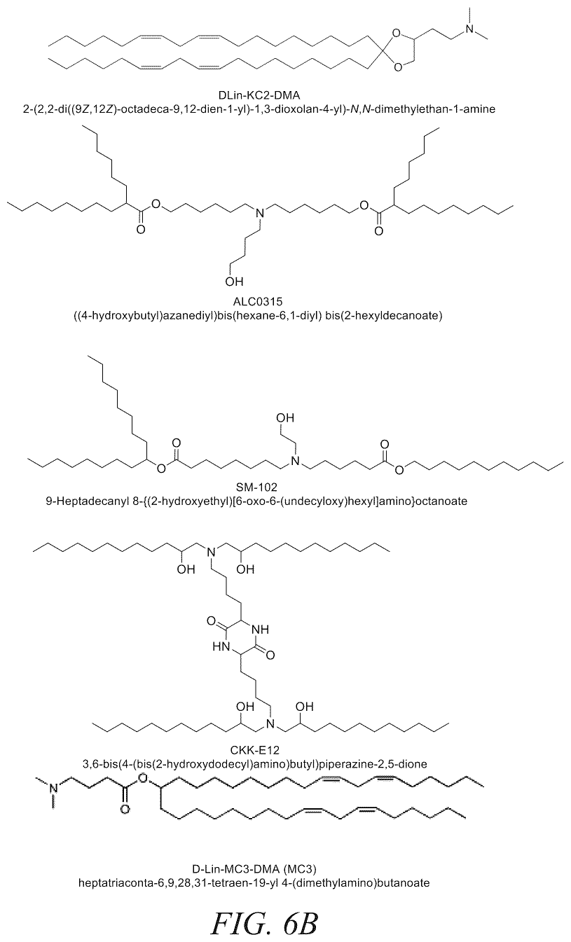

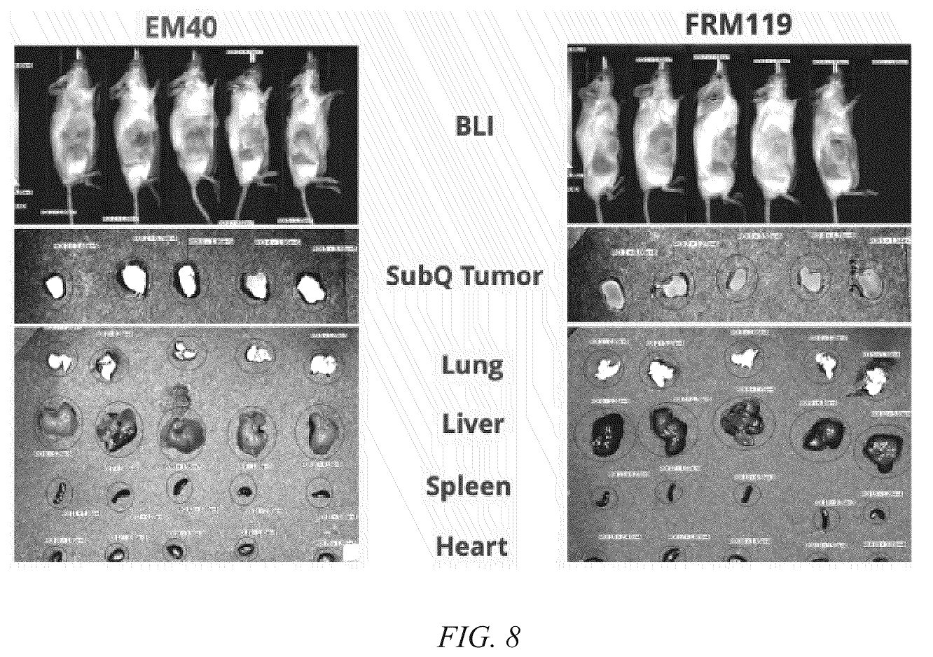

FIG. 8 demonstrates decreased liver and non-tumor expression driven by adding surface modification of LNP formulations described herein. Shown are photographs of BLI imaging of tumor and other organs from mice administered LNPs as in FIG. 6 showing that LNP formulations incorporating increased concentrations (e.g., 3 mole %) of a PEG-lipid bearing two saturated acyl chains (e.g., two saturated C18 acyl chains, “FRM119”) show liver expression near the lower limit of detection compared to compositions bearing lower concentrations of PEG-lipids (e.g., 0.75%) with two saturated acyl (e.g., C14 acyl or fatty acyl) chains (“EM40”).

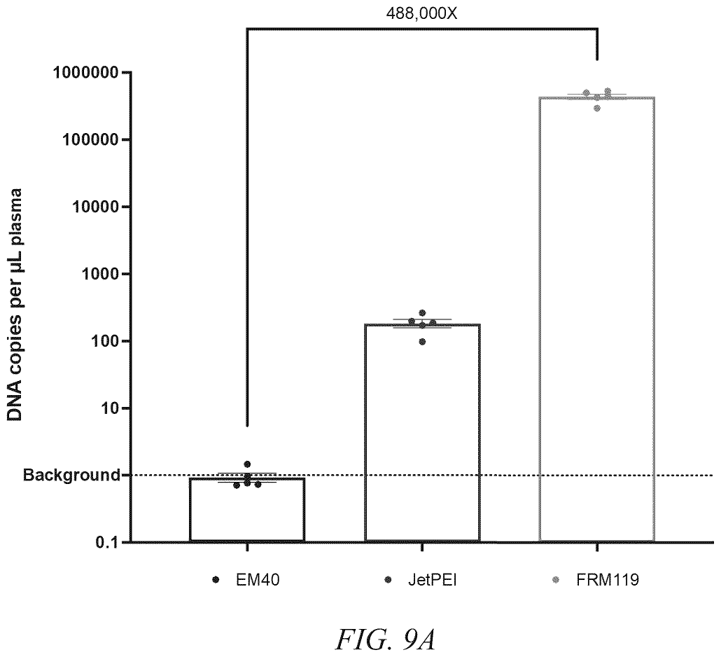

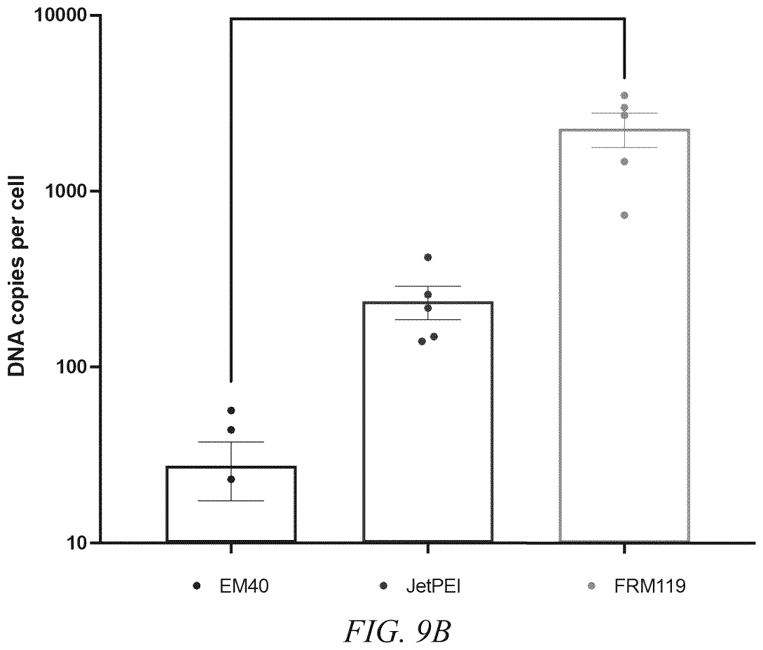

FIGS. 9A and 9B demonstrate that surface modification of LNP formulations described herein (“FRM 119”) results in enhanced levels of LNPs in serum 48 hr after injection into mice, and enhanced levels in tumors. FIG. 9A shows a graph of DNA copies per μL plasma assessed in mice by qPCR 48 hours after intravenous injection. FIG. 9B shows a graph of DNA copies per cell in dissected tumors from corresponding mice 48 hours after administration.

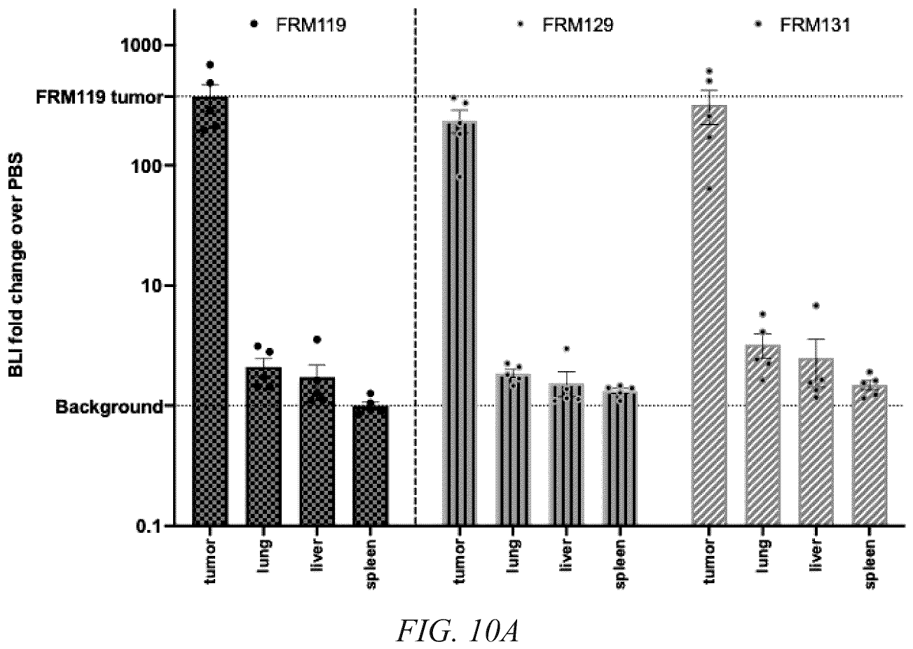

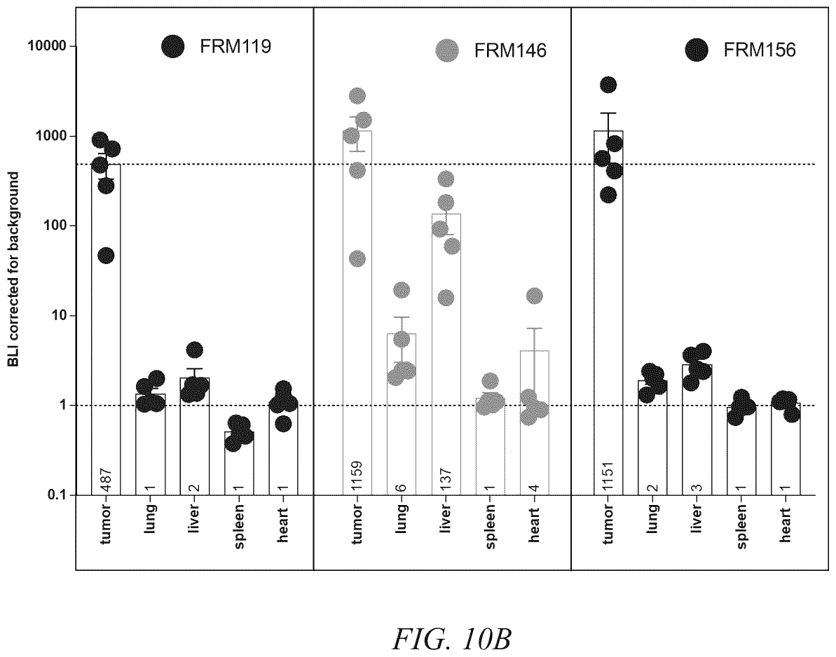

FIGS. 10A and 10B demonstrate that LNP formulations described herein (“FRM 119”, “FRM129”, “FRM131”, “FRM146”, and “FRM156”) can result in selectivity of tumor vs liver targeting. Shown is a graph of BLI imaging of dissected organs 48 hours of injection into a mouse H1299 xenograft model. FIG. 10A shows tissue selectivity for FRM119, FRM 129, and FRM131,while FIG. 10B shows tissue selectivity for FRM119, FRM146, and FRM156.

### Toxicity

Acute toxicity due to administration of LNPs was assessed by clinical chemistry and hematological analysis of serum samples obtained at various time-points following a single administration of DNA-LNPs. The clinical chemistry assessments included measures of liver enzymes such as Alanine Aminotransferase (ALT) and Aspartate Aminotransferase (AST), which are released into the blood following hepatocellular toxicity upon drug administration. Therefore, serum AST levels are often used as a preliminary measure of liver toxicity. In addition, the potential of DNA-LNP administration to stimulate an acute inflammatory response was also assessed by measuring the serum levels of cytokines such as TNF-alpha, IL-6, and MCP-1 at 2-4 hours after dosing. These cytokines are messenger molecules in the immune system and elevations in their serum levels upon dosing are indicative of an acute innate immune response to the drug product administration.

### Example 4.—Evaluation and Engineering of Surface Modifications to LNPs for Circulatory Stability

The overall goal of this study was to develop an in vitro screening platform for surface-engineered lipid nanoparticles (LNPs) exhibiting long circulation in the bloodstream. A library of LNPs with varying surface properties was prepared and evaluated in vitro to establish parameters for predicting their pharmacokinetics in vivo. The screening platform focused on building in vitro-in vivo correlations by addressing two key factors:
(1) Can the effect of in vivo protein corona (e.g., biological identity) be incorporated into an in vitro screening assay for LNPs?
(2) Can the circulation half-life of LNPs in vivo be predicted through cellular uptake in vitro?

### Overview of Surface Engineering Pipeline and Evaluation of Plasma-Incubated LNPs

The protein corona is a layer of proteins that can adsorb onto the surface of lipid nanoparticles (LNPs) upon contact with biological fluids such as blood or plasma. This protein corona can significantly affect the biological behavior of LNPs, including their cellular uptake, biodistribution, and pharmacokinetics. Based on the chemical identity of the LNP surface, the adsorbed proteins can construct a ‘biological identity’ that determines the subsequent interaction of the LNP with the immune system and both target as well as non-target cells. It was postulated that in vitro evaluation of LNPs with their true biological identity might provide a better representation of their in vivo behavior. To understand this effect, two LNPs were formulated with varying polymer loading of DMG-PEG2000 as described below.

| | | | | DMG- |
|---|---|---|---|---|
| | MC3, | SOPC, | Cholesterol, | PEG2000, |
| Formulation | mole % | mole % | mole % | mole % |
| PEG-1.5% | 50 | 10 | 38.5 | 1.5 |
| PEG-3% | 50 | 10 | 37 | 3 |

The uptake in H1299 cells was evaluated using Cy5-labeled DNA. LNP with 3% PEG was observed to show lower uptake than 1.5% PEG formulation. This result was consistent with the expectation that the more strongly shielded surface—the chemical identity—formed by 3% PEG would resist non-specific interactions with biological membranes and therefore have poorer uptake into cells. However, after incubating the LNPs with CD1 mouse plasma for 1 hour at 37° C., we observed very similar uptake for both the formulations This result suggest that the PEG coating was rapidly desorbed or replaced by adsorbed proteins from mouse plasma, leading to a very similar biological identity. Consequently, both these LNPs were predicted to have very similar uptake in vivo upon acquiring the protein corona, which would not be predicted by the in vitro result in the absence of plasma pre-incubation. Therefore, the effect of different surface engineering approaches only after pre-incubating LNPs with mouse plasma was evaluated, to ensure that our assay captured the biological identity of the LNPs rather than merely the chemical identify of the surface.

### Evaluation of LNP Library in H1299 Cells

The addition of PEG (polyethylene glycol) to LNP formulations can modify the composition and structure of the protein corona, which can help to control the biological fate of the particles. PEGylation can prevent the adsorption of certain proteins onto the surface of LNPs, which may reduce the clearance of the particles by the immune system and prolong their circulation time in the bloodstream. However, the presence of PEG can also alter the protein corona in ways that may affect the efficacy of LNPs, including their ability to be taken up by cells. Therefore, the optimization of PEGylation conditions and the selection of appropriate lipid-PEGs were evaluated in the development of effective LNP systems.

PEG-lipids comprise three components—the lipid tail, linker, and PEG polymer. The lipid tail is understood to interact with the internal core components of the nanoparticle and act as an anchor for the PEG-lipids. The lipid tail can vary based on the length (number of CH2 units) of each tail (which can further be symmetric or asymmetric) and the degree of unsaturation (saturated, monounsaturated, polyunsaturated). These variations in the lipid tail can have direct effects on the anchoring strength of the lipid-PEGs. The linker component primarily comprises an extended segment of the lipid tail and connects it with the polymer component. These linkers can vary based on their chemical identity, length, charge and degradability. The PEG segment is the hydrophilic component, which is oriented and exposed outside the LNP, and act as barrier between the LNP and the serum components. The polymer can further have several variations in factors such as chain length, branching or end-chain functionalization.

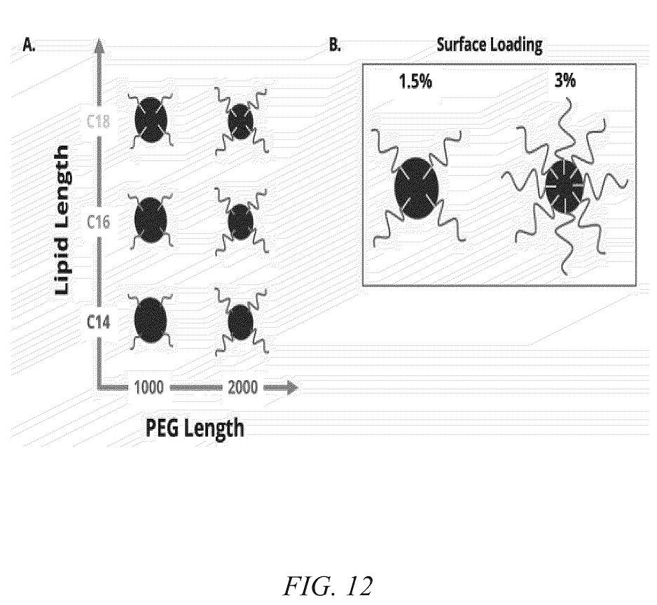

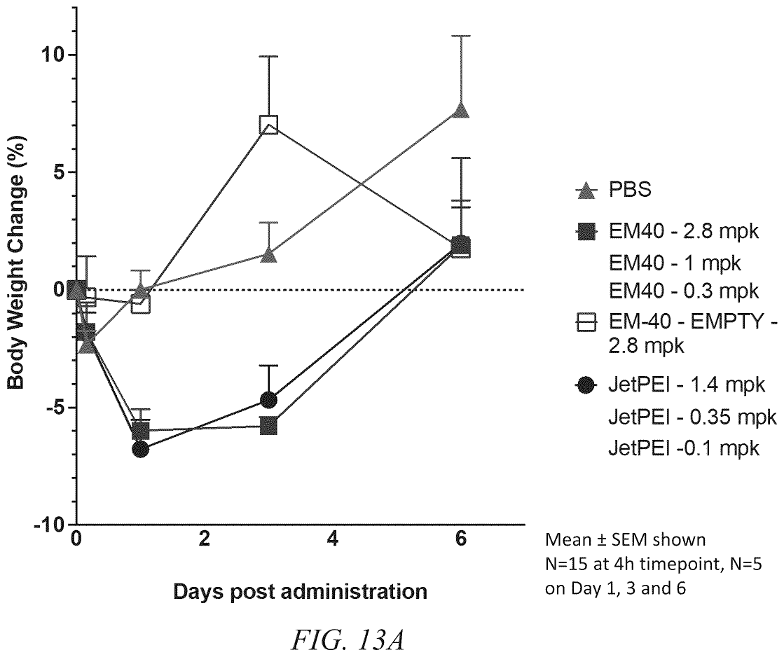

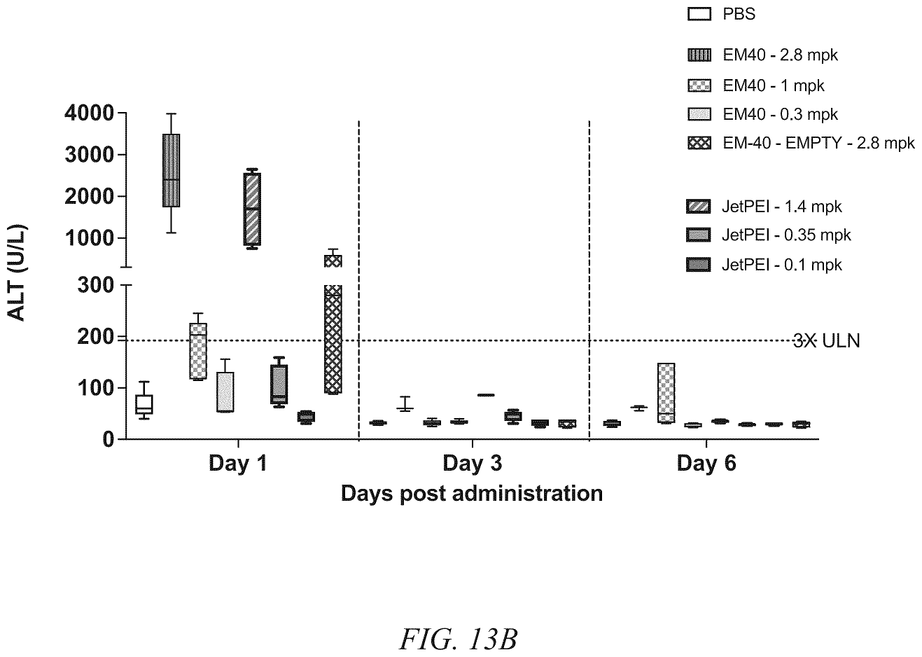

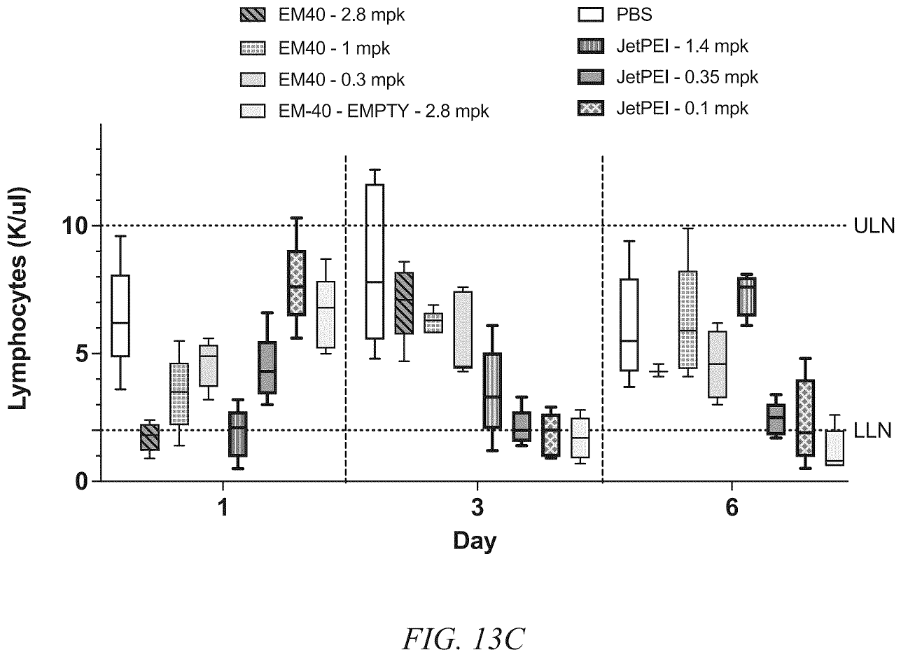

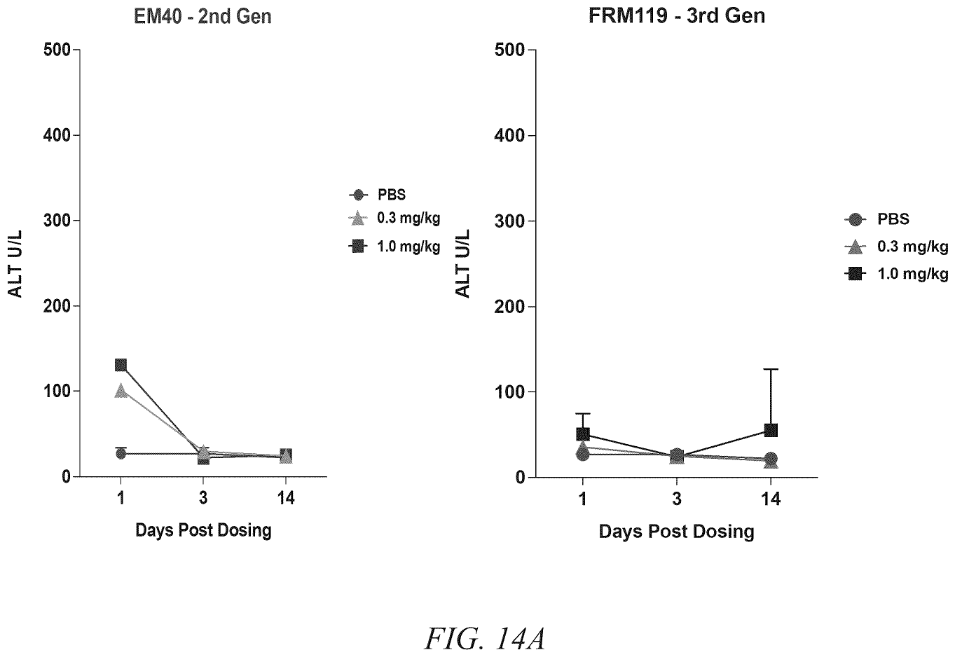

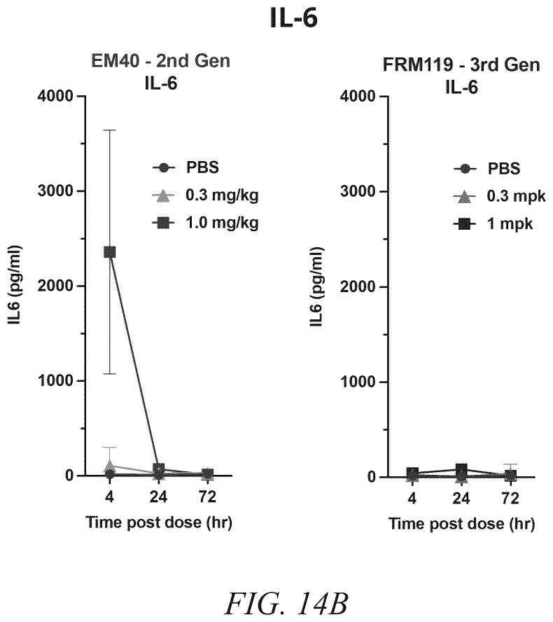

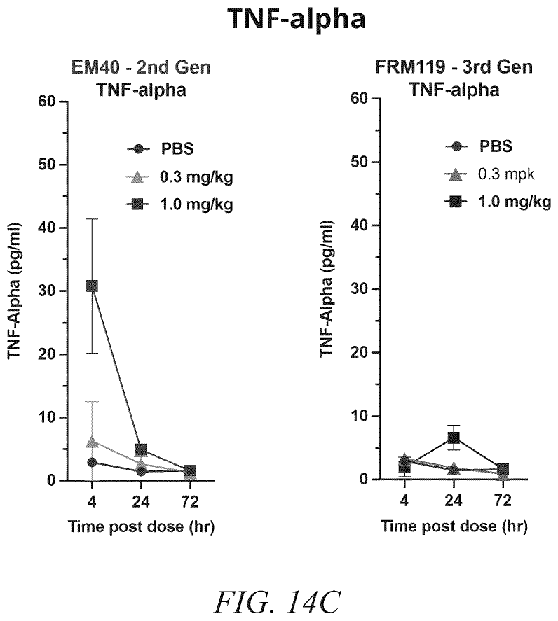

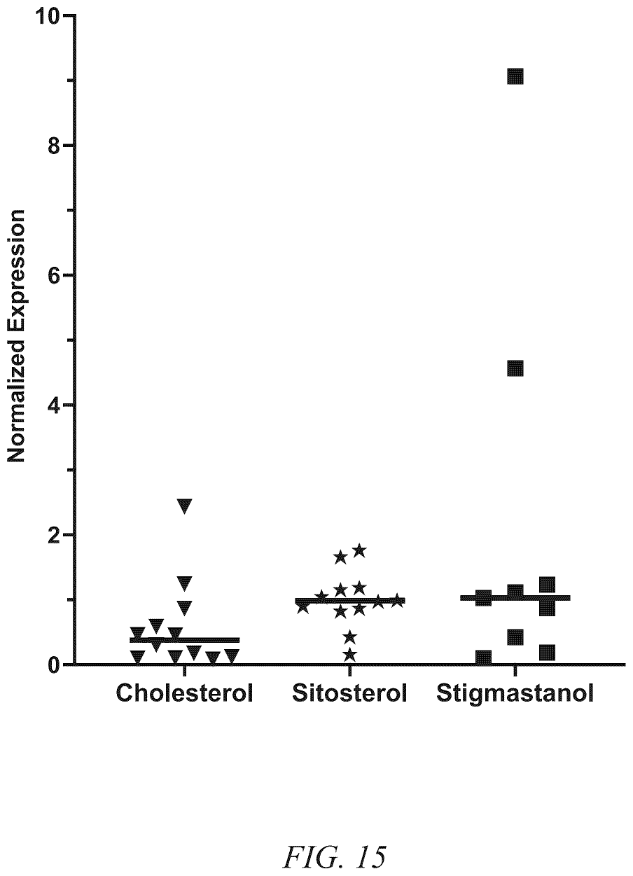

To elucidate the effect of different components of PEG-lipids-lipid tail length, PEG length and the overall surface composition, an LNP library was developed using different PEG-lipid variants (FIG. 12). Three chain lengths of saturated, symmetric di-lipid tails—C14, C16, and C18—were used to vary the anchoring strength of the PEG-lipids. The hydrophobicity of PEG-lipids increased with the length of lipid tails, hence predicted to lead to stronger interaction with the lipid nanoparticles. Each of these lipid tails were combined with two different molecular weights of PEG polymer—1000 or 2000. The molecular weight (or length) of the polymer was thought to define the hydrophilic nature of lipid-PEGs and larger MWs were understood to be more hydrophilic in nature. Hence, by varying the lipid tail length and polymer length, the relative amphiphilicity of the lipid-PEGs was thought to be varied. The library was further expanded to vary the surface coverage of the polymers through two molar compositions of the lipid-PEGs—1.5% and 3%.

H1299 cells were treated with the library of LNPs overnight and the cells were analyzed using flow cytometry. The uptake of LNPs was quantified based on Cy5 dye signal in the H1299 cells and the expression was quantified using the GFP signal. The geometric mean fluorescence intensity (GMFI) of Cy3+ and GFP+cell populations were used to represent each LNP as a point on a plot correlating uptake with expression. This plot was termed the “Uptake-Expression Correlation Plot”.

### Effect of Lipid Tail Lengths

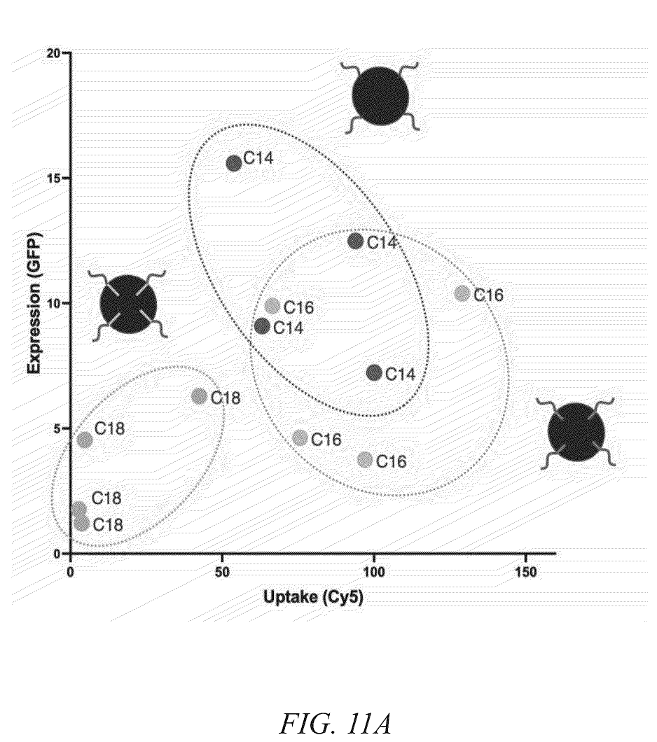

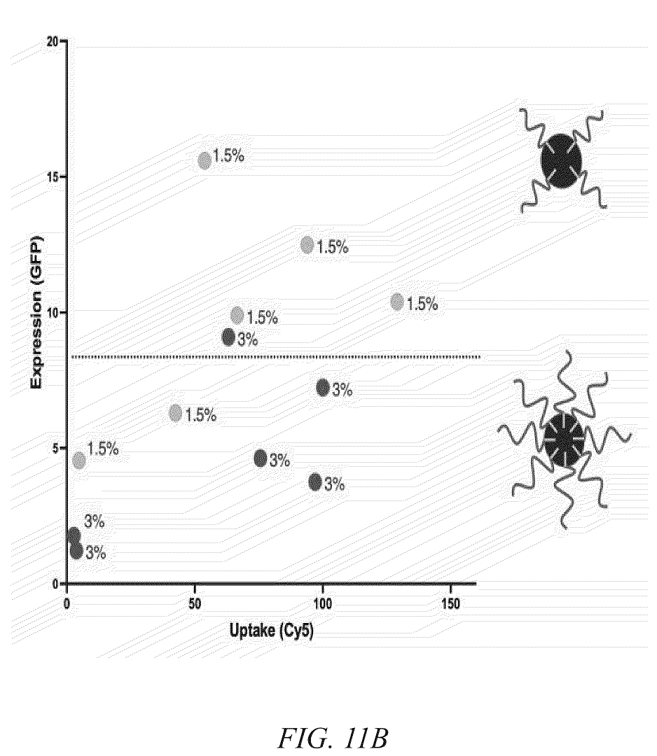

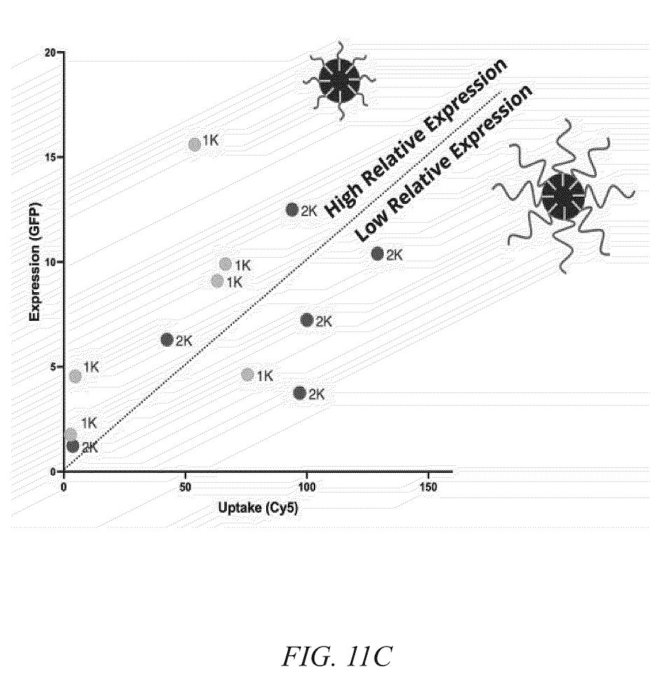

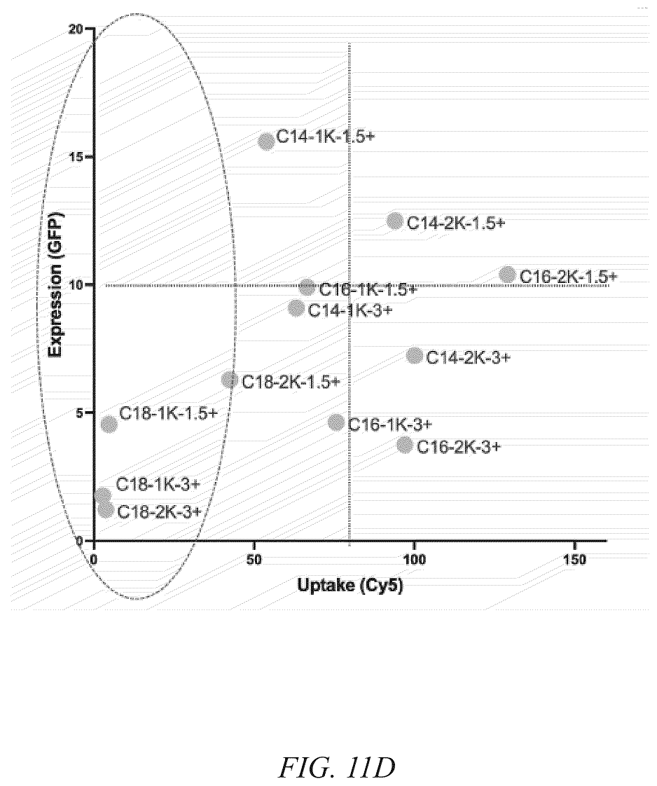

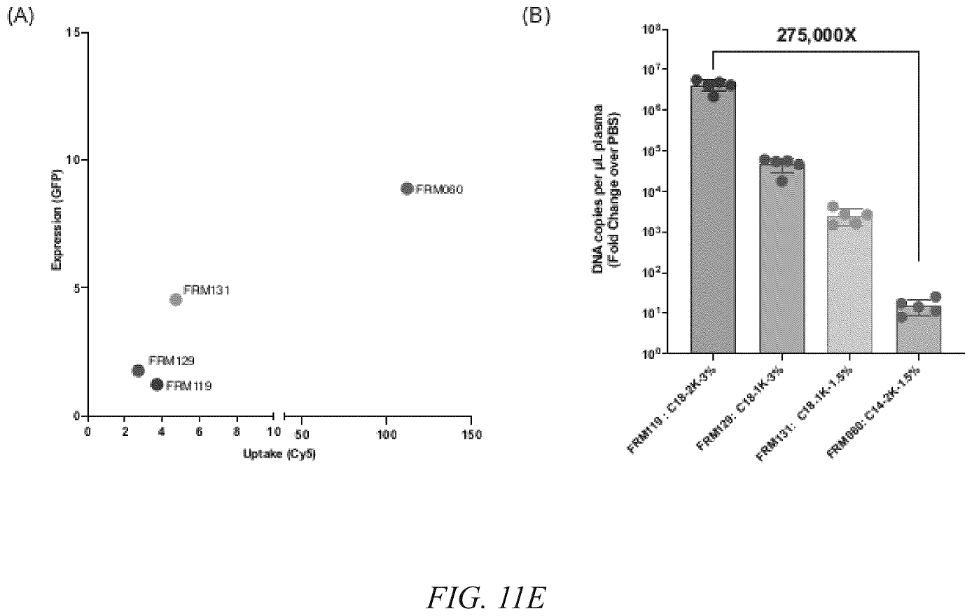

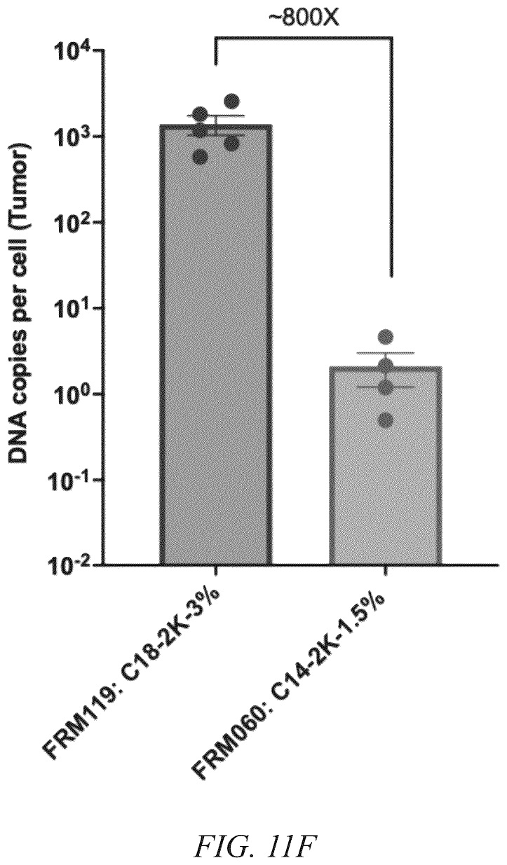

It was observed that the length of the lipid tail affected both uptake and expression of the LNPs (FIG. 11A). LNPs with C18 lipid tails significantly reduced the overall uptake and expression of the nanoparticles. While the LNPs with C14 and C16 lipid tails showed higher uptake than C18 lipid tails, the expression was observed to be higher for C14 lipids. These trends led to the understanding that C18 lipids were better at reducing uptake, while C14 tails were better at relative expression.

These results suggested that relative hydrophobicity played the most significant role in the uptake of LNPs. The longer tails likely were thought to not dissociate from the surface and the PEG was thought to continue to provide a protective cloak around the LNPs, which would reduce the extent of uptake into cells. Another interesting result was the relative transfection efficiency of C16 and C14 tails. The result that despite having similar uptake, C14 tails are better at expression than C16 tails, suggested that tail length played an additional role in endosomal release of LNPs. Shorter tailed lipids were thought to greater chances of surface desorption once the LNP is in the endosome and hence were thought to thereby allow for higher expression than longer tails.

### Effect of Surface Loading

Next, the trends for varying surface loading on the performance of LNPs were assessed. The surface loading of PEG-lipids on LNPs was observed to affect both uptake and expression (FIG. 11B). Irrespective of lipid length or polymer length, high surface loading (3%) led to relatively lower expression in comparison to 1.5% percent composition. This was attributed to the potential interference of PEG shielding on the endosomal escape process—e.g., that higher PEG loading on the surface resulted in a persistent PEG shield that could prevent fusion of the LNP lipids with the endosomal membrane and subsequent release of the cargo.

### Effect of PEG Length

Finally, the effect of polymer length on the performance of LNPs was assessed. PEG polymers with MW of 2000 led to lower relative expression as seen in FIG. 11C. While the uptake into cells varied depending on lipid length and surface loading as described above, PEGs with MW of 1000 were found to be better at relative higher expression. This was consistent with the effect of increased PEG loading on expression detailed above: e.g., that increased PEG shielding due to a longer PEG polymer length, or greater loading on the surface, could interfere with endosomal escape and membrane fusion processes and reduce overall gene expression.

### Overall Trends of LNP Library

The combined Uptake-Expression Correlation Plot (FIG. 11D) shows the uptake and expression from all the LNPs in the library. This plot combines the effect of several modifications that affect the biological identity of the LNP surface, as explored through a library of lipid-PEGs with varied tail length, polymer length, and surface loading, in a single comprehensive assessment of the cellular outcomes.

It was hypothesized that cellular uptake—facilitated by the biological identity of the LNP, where the adsorbed protein corona mediates interactions with cell-surface receptors and uptake pathways—would correlate with in vivo liver uptake and clearance of LNPs. Therefore, LNPs that exhibit reduced cell uptake in vitro are less likely to be taken up and cleared by phagocytic cells in the liver and spleen and would therefore be more likely to have longer circulation half-lives and extended pharmacokinetics. This was hypothesized to lead to greater accumulation of LNPs in tumors upon eventual extravasation from the bloodstream.

However, once the LNPs exit the blood and accumulate in tumors, it was also beneficial to the stated purpose to have strong gene expression within tumors cells, which would lead to greater ‘signal’ from a payload.

These combined arguments led to the construction of the hypothesized ‘target profile’ of the LNPs, as depicted by the dotted oval in the plot in FIG. 11D. These LNPs would be predicted to avoid rapid in vivo clearance due to the low cellular uptake, but also generate high gene expression upon eventual internalization into tumor cells.

As described above, the uptake of nanoparticles was thought to be primarily driven by the formation of protein corona and resulting interactions with the cellular membrane. Since PEG was thought to act as a protective layer, the surface shedding of PEG was thought to be crucial for the formation of protein corona on LNPs. Hence, the relative position of an LNP on the X-axis could be interpreted in terms of overall shedding rate of the lipid-PEG component incorporated in that LNP. The table below shows the trend for the surface shedding of each lipid-PEG based on their respective uptake profile.

| | Surface Shedding Rate | Endosomal Release |
|---|---|---|
| Trend | (Based on uptake) | (Based on expression) |
| 1 | C16-2K | C14-1K |
| 2 | C14-2K | C14-2K |
| 3 | C16-1K | C16-1K |
| 4 | C14-1K | C16-2K |
| 5 | C18-2K | C18-1K |
| 6 | C18-1K | C18-2K |

Further, the expression depended on the cellular internalization and endosomal release thereafter. The relative position of LNPs on the Y-axis should depend on the efficiency in endosomal release processes. The table above shows the trend of endosomal release for each lipid-PEG that was evaluated in the library.

In order to correlate the trends from Uptake-Expression Correlation Plot with in vivo performance, four formulations (FIG. 11E panel a) representing different uptake and expression profiles were evaluated in a subcutaneous xenograft tumor model established with H1299 human lung cancer cells in an immune-compromised NSG mouse host. Pharmacokinetic (PK) analysis was performed for vector copy numbers, e.g., nanoplasmid DNA copies in the plasma to understand the effects on blood circulation. For this preliminary analysis, a single timepoint at 48 h post administration of LNPs was analyzed. The quantity of CAG-FLUC nanoplasmid DNA in plasma or tumor tissue was measured using qPCR assays specific to the nanoplasmid DNA sequence.

As shown in FIG. 11E, LNPs with C18 lipid tails were found to be long circulating with 10³-10⁷ DNA copies in the plasma at 48h. On the contrary, C14 lipid tail led to rapid clearance from circulation, resulting in very low DNA copies in plasma. These results were found to be in good correlation with the in vitro trends in cell uptake from the correlation plot. LNP with C14 lipid tail represents a commercial-like formulation and these results are consistent with the low circulating nature of the formulation. Among the LNPs with C18 lipid tails, it was observed that the DNA copies dropped significantly as the surface coverage was reduced by either shorter polymer length or overall surface loading. The relative shift in the uptake of LNPs—C18-1K-3% and C18-1K-1.5%, was also observed to be well-correlated between the correlation plot and the in vivo PK trends.

Next, the effect of varying circulation half-life of LNPs was evaluated by using PK assays on tumor tissue. It was hypothesized that longer circulation would allow nanoparticles to not only avoid rapid liver uptake but also provide a greater opportunity to accumulate in tumor tissue due to the leaky vasculature in tumors permitting extravasation of nanoparticles from circulation over time. Consistent with this hypothesis, LNPs with C18 lipid tails accumulated 10-1000× higher copies of nanoplasmid DNA in the tumor (FIG. 11F). Specifically, C18-2K-3% (FRM119) with the highest DNA copies in plasma (FIG. 11E) resulted in over 1000 copies of the DNA in the tumor. FRM129 and FRM131 with relatively shorter half-life showed less DNA copies in the tumor compared to FRM119. Furthermore, C14-2K-1.5%, an commercial-like LNP formulation with C14 lipid tail accumulated approximately 1 DNA per cell. These results were found to be in good correlation with the PK in plasma and supported the hypothesis that improved tumor PK is promoted by improving plasma PK.

### Example 5.—Flexible Surfaces Achieve a Balance of Long Circulation and High Transfection

It was hypothesized that reducing LNP clearance by the reticulo-endothelial system would allow for extended blood circulation and increase the likelihood of delivery to tumors in distal organ sites. Therefore, a large library of polymer-lipids was screened to identify LNPs capable of transfecting cells in vitro, but having relatively low cell uptake, potentially indicating lower opsonization by plasma proteins. One of these formulations (FRM119) achieved long circulation in mice but resulted in relatively poor transfection in H1299 cells in vitro. We then further modified the FRM119 formulation to include lipids with flexible surfaces (e.g., FRM146).

To evaluate the performance of formulations with flexible surfaces, an in vitro assay of the new formulations was first performed. H1299 cells were treated with 1-10 pg/cell CAG-GFP DNA-LNP of FRM146 alongside FRM001, FRM055, and FRM060 (patisiran-like MC3 LNP compositions that differ only in helper lipid type and DMG-PEG2000 mol %) and the transfected cells were analyzed after 24 h by flow cytometry as described in Example 4.

Alongside the in vitro assay, H1299 subcutaneous (SQ) tumors in NSG mice were intravenously (i.v.) dosed with DNA-LNP of FRM060, FRM119, and FRM146 at 1.4 mg/kg and analyzed after 48 h as described in Example 3.

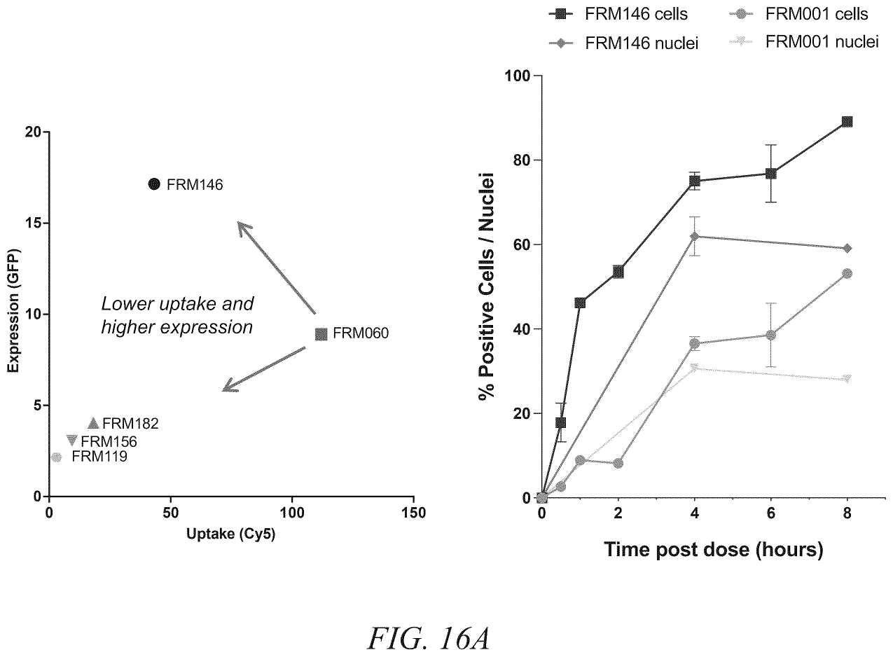

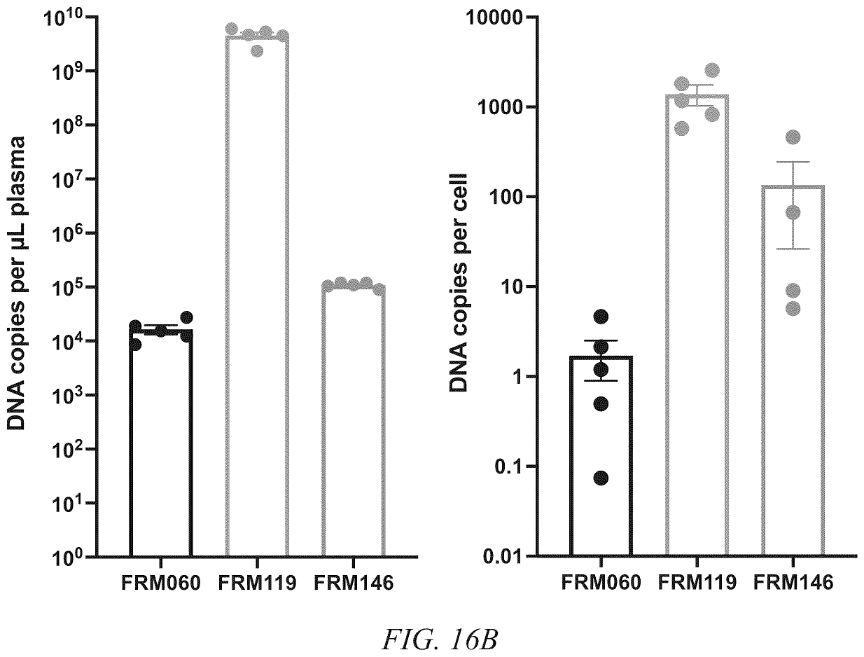

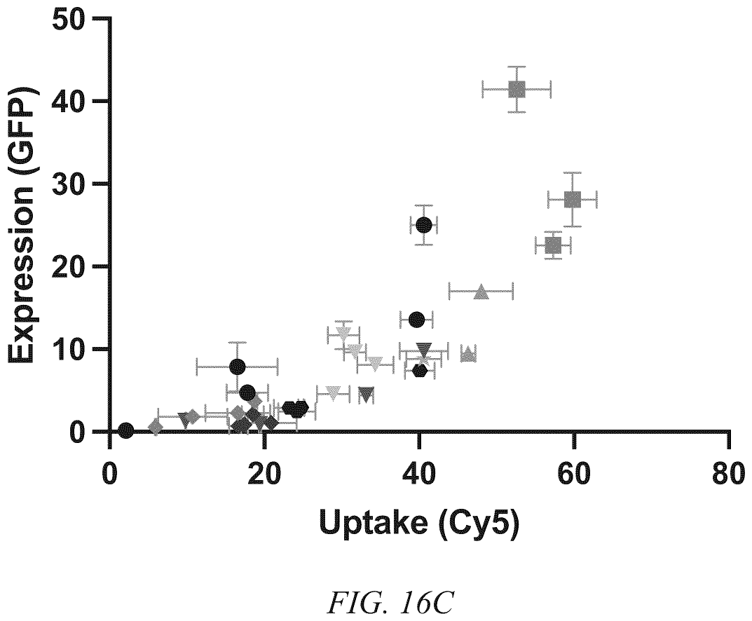

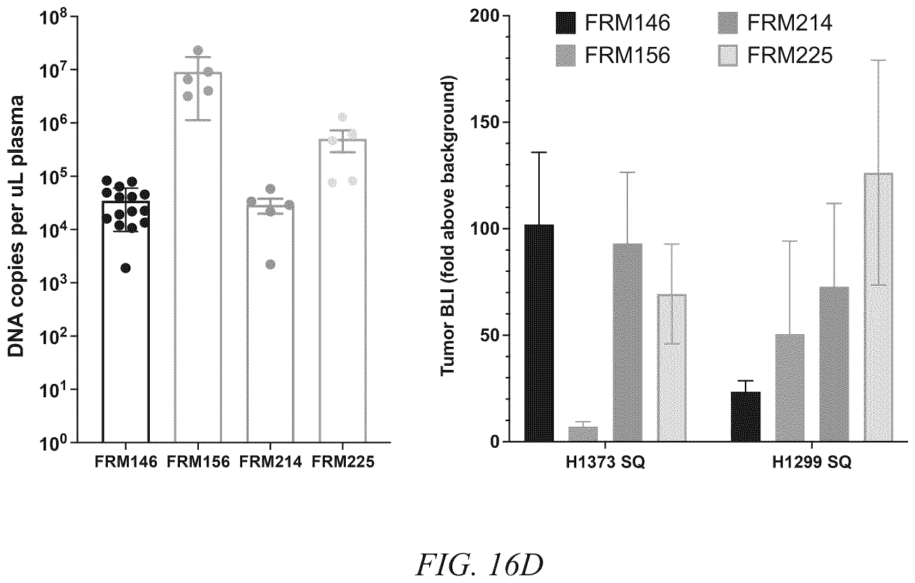

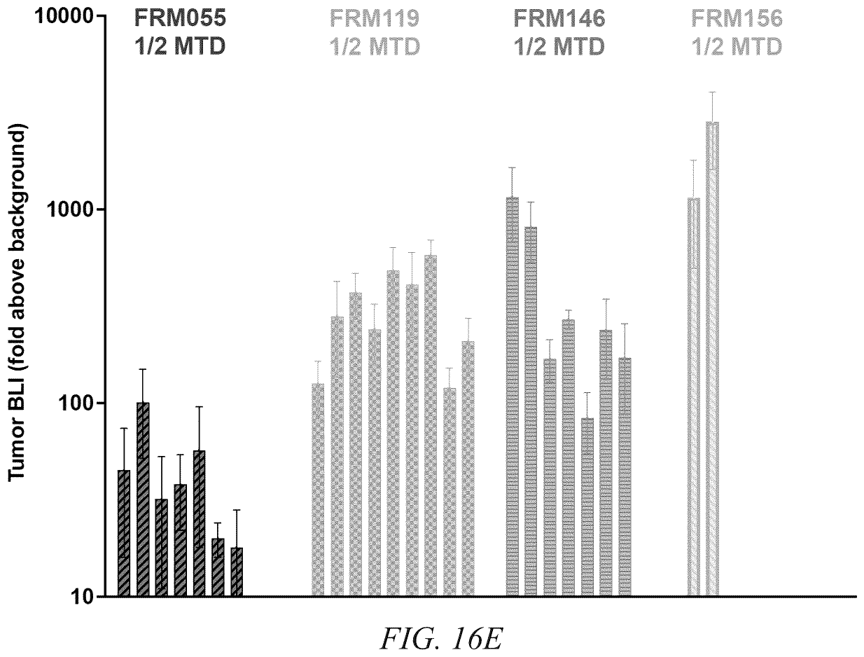

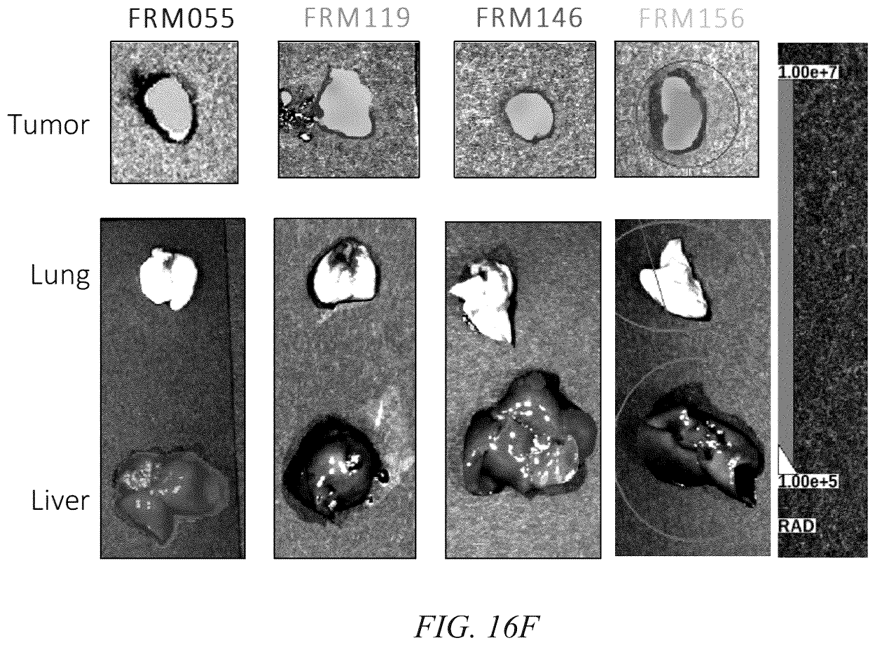

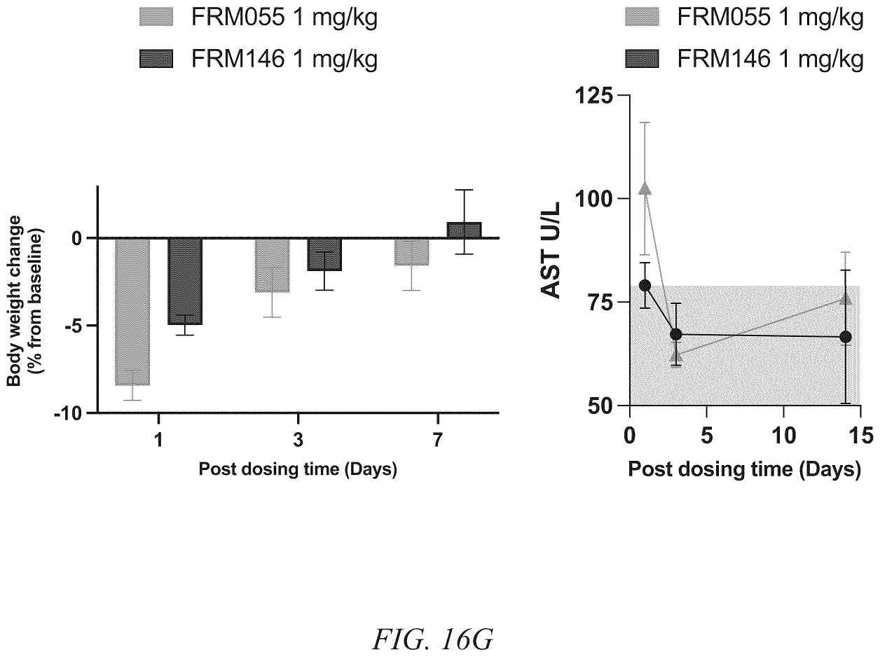

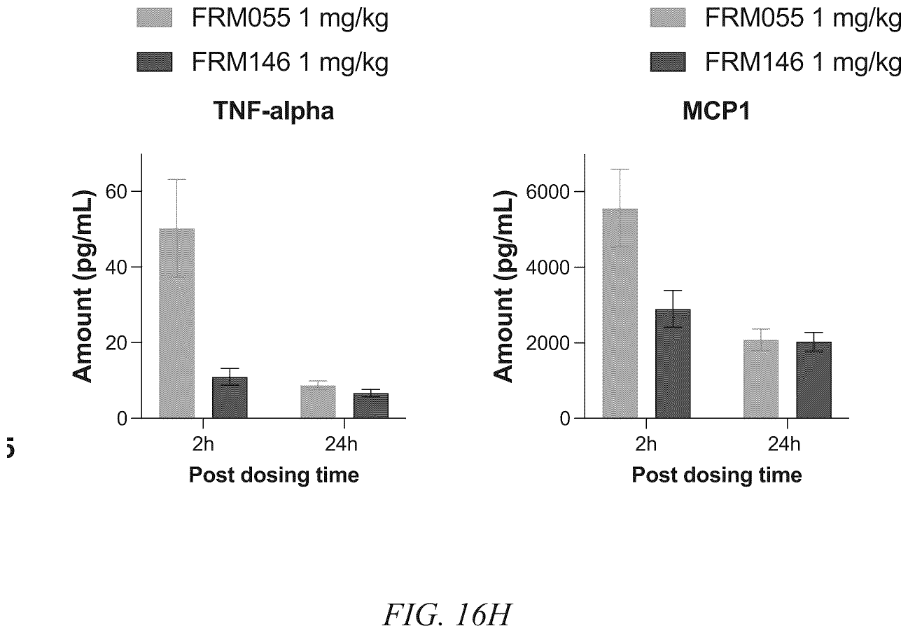

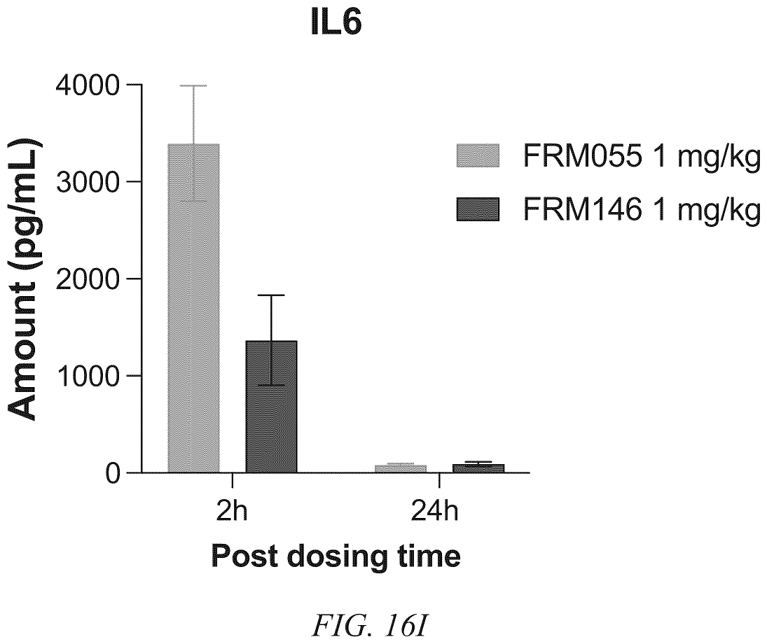

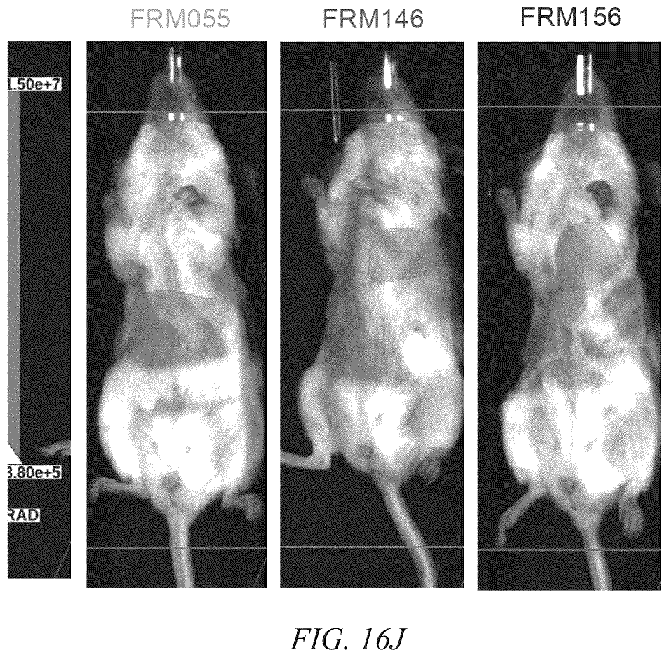

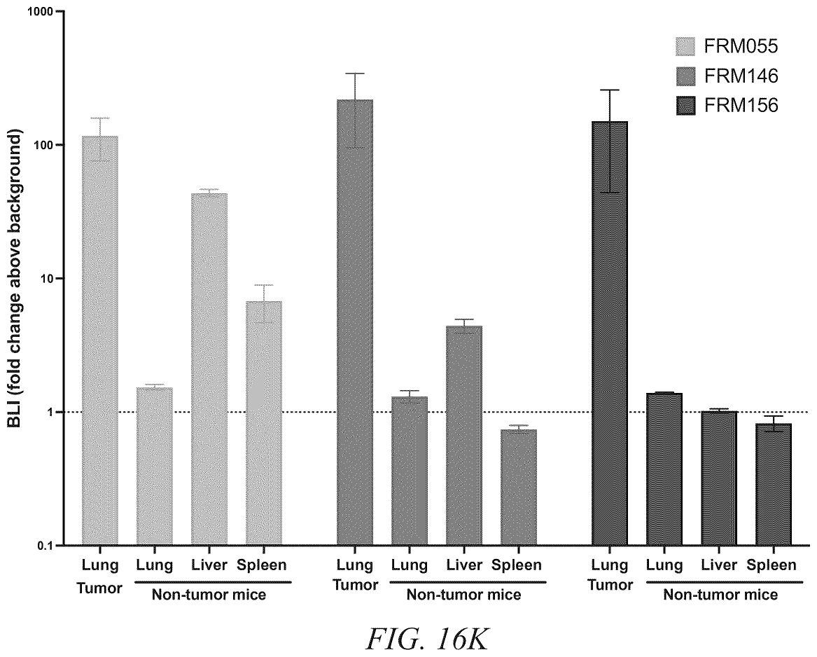

The results are shown in FIG. 16A and FIG. 16B. FRM146 exhibited more rapid cell uptake and nuclear delivery in vitro (see FIG. 16A, right panel), potentially due to more dynamic rearrangements of the polymer-lipid inside cells. In addition, FRM146 exhibited greater GFP expression in vitro (see FIG. 16A, left panel) despite lower cell uptake than FRM060, the reference formulation based on a commercial composition. This lower cell uptake correlated with increased plasma PK/stability (FIG. 16B, left panel) and increased tumor uptake of DNA (see FIG. 16B, right panel), verifying the ability of longer-circulating LNPs like FRM 146 to deliver more DNA to tumors.

TABLE 1D
LNP Compositions According to Example 5.

| | FRM001 | FRM182 |
|---|---|---|
| DLin-MC3-DMA, mole % | 50 | 50 |
| Cholesterol, mole % | 38.5 | 37 |
| DSPC, mole % | 10 | — |
| SOPC, mole % | — | 10 |
| DMG-PEG2k, mole % | 1.5 | — |
| DSG-PEG2k, mole % | — | 1.5 |
| DOPE-PEG1k, mole % | — | 1.5 |

### Example 6.—DoE-Driven Core Engineering to Further Increase Transfection of Long-Circulating LNPs

FRM156 was identified as an LNP with a flexible surface that balanced long circulation with the ability to transfect cells. Further improvements in transfection were obtained by optimizing core lipid components of FRM156 such as sterols and helper lipids that are known to influence the efficiency of LNP endosomal escape. High-throughput screening (see FIG. 16C) identified several compositions that produced significant increases in GFP expression compared to FRM156 in vitro, resulting in the discovery of formulations FRM214 and FRM225.

To observe the performance of these formulations H1299 cells were treated with 1-10 pg/cell CAG-GFP DNA-LNP and analyzed after 24 h by flow cytometry as described in Example 4.

Additionally, a bilateral SQ model with lung H1373 and H1299 xenografts in NSG mice was intravenously (i.v.) dosed with CAG-FLUC DNA-LNP at 1.4 mg/kg and analyzed after 48h, as described in Example 3.

The results of these experiments are shown in FIG. 16D. The left panel of FIG. 16D shows plasma PK of the formulations in mice upon dosing with the named LNP formulations, while the right panel of FIG. 16D shows bioluminescent intensity of tumors transfected with the named LNP formulations. While some of these formulations such as FRM214 reduced in vivo plasma PK, further improvements to the formulation (e.g., FRM225) rescued these effects and resulted in strong DNA expression in multiple lung cancer xenograft tumor models in mice.

TABLE 1E
LNP Compositions According to Example 8.

| | FRM214 | FRM225 | FRM156 |
|---|---|---|---|
| DLin-MC3-DMA, | 45 | 45 | 50 |
| mole % | | | |
| SOPC, mole % | 0 | 0 | 10 |
| DSPC, mole % | 16 | 16 | 0 |
| Stigmastanol, mole % | 36 | 18 | 0 |
| Cholesterol, mole % | 0 | 18 | 37 |
| DSG-PEG2000, mole % | 2 | 2 | 2 |
| DOPE-PEG1000, mole | 1 | 1 | 1 |
| N/P ratio | 5.21 | 5.79 | 5.79 |
| Mass ratio (Total | 22.31 | 24.6 | 21.5 |
| Particle size (nm) | 82 | 99 | 80 |
| Zeta potential (mV) | −6.5 | −2.1 | −7.7 |

### Example 7.—Engineered LNPs Reduce Liver Expression and Produce >10-Fold Increase in Tumor Expression

LNPs engineered for long circulation were evaluated for gene expression and biodistribution in mouse tumor models.

H1299 subcutaneous (SQ) tumors in NSG mice were intravenously (i.v.) dosed with CAG-FLUC DNA-LNP at one-half of the maximum tolerated dose (½ MTD).

After 48h, tumors and other organs were excised and ex vivo bioluminescence imaging (BLI) was performed to determine luciferase reporter gene expression as described in Example 3.

The results of the experiment are shown in FIG. 16E and FIG. 16F. FIG. 16E shows a chart of BLI tumor fluorescence above background for each LNP injected into mice. Each bar represents an individual experiment with at least 5 mice, demonstrating that the trends were consistent across repeated experiments. FIG. 16F shows representative luminescent images of dissected organs (tumor, lung, liver) from mice transfected as in FIG. 16E. Strikingly, we found that FRM119, FRM146, and FRM156 not only produced 10-fold or greater tumor DNA expression than patisiran-like MC3 LNPs like FRM055 (see FIG. 16E), but also dramatically reduced liver DNA expression (see FIG. 16F), resulting in a significantly increased tumor-to-liver signal-to-noise ratio (SNR) for those compositions.

### Example 8.—Engineered LNPs are Better Tolerated and have Lower Immune Stimulation than Liver-Tropic LNPs

We hypothesized that altering LNP biodistribution and clearance mechanisms could alter the tolerability of LNPs in vivo due to reduced uptake by liver and immune cells. Accordingly, we assessed acute toxicity of FRM146 with FRM055 as a comparison. Acute toxicity due to administration of LNPs was assessed by clinical chemistry and hematological analysis of serum samples obtained at various time-points following a single administration of DNA-LNPs. The clinical chemistry assessments included measures of liver enzymes such as Aspartate Aminotransferase (AST), which is released into the blood following hepatocellular toxicity upon drug administration. Therefore, serum AST levels are often used as a preliminary measure of liver toxicity. In addition, the potential of DNA-LNP administration to stimulate an acute inflammatory response was also assessed by measuring the serum levels of cytokines such as TNF-alpha, IL-6, and MCP-1 at 2-4 hours after dosing. These cytokines are messenger molecules in the immune system and elevations in their serum levels upon dosing are indicative of an acute innate immune response to the drug product administration.

The results of this experiment are shown in FIG. 16G, FIG. 16H, and FIG. 16I, which show body weight and serum AST, TNF-alpha and MCP1, and IL-6, respectively for FRM146 and FRM055. It was observed that LNPs with flexible surfaces such as FRM146 trended towards lower body weight loss, lower elevations of liver enzymes such as AST, and lower immunotoxicity as measured by acute cytokine elevations post i.v. DNA-LNP administration.

### Example 9.—Engineered LNPs Produce Strong DNA Expression in Tumor-Bearing but not Normal Lungs or Liver

The ability of engineered LNPs FRM055, FRM146, and FRM156 to deliver DNA to lung tumors was evaluated in an orthotopic H1299 xenograft model.

NOD.Cg-Prkdc scid Il2rg tm1Wjl/SzJ mice (NSG, stock 005557, Jackson Laboratory) were used at 7-10 weeks of age at inoculation. The left flank of the mice was shaved 1-3 days prior to inoculation. On the day of in vivo tumor inoculation, mice were anesthetized under isoflurane (2-4%) and placed in the left lateral decubitus position. 1.5×106 H1299 cells resuspended in 20 uL of PBS:Matrigel (1:2) were percutaneously injected into the upper margin of the sixth intercostal rib on the left anterior axillary line to a depth of about 2-4 mm. Control tumor-free mice received 20 uL of PBS:Matrigel. Tumor volume was monitored and randomized by CT imaging.

Approximately 4-5 weeks after tumor inoculation, tumor-bearing and control mice were dosed intravenously (i.v.) with CAG-FLUC DNA-LNP at 1.4 mg/kg. After 48h, whole-body and ex vivo BLI imaging were performed to measure luciferase gene expression as described in Example 3.

The results of the experiment are depicted in FIG. 16J and FIG. 16K. FIG. 16I shows representative BLI images of mice dosed with the named LNPs 48 hours after intravenous dosage. FIG. 16K shows BLI intensity of individual organs after gross necropsy for mice dosed with the named LNPs 48 hours after intravenous dosage. It was observed that LNPs with flexible surfaces like FRM146 and FRM156 showed strong expression in tumor-bearing lungs, but dramatically lower expression in non-tumor bearing lungs or liver.

### Example 10.—Evaluation of Mixed Surfaces/Conjugated Lipids in LNPs

It was hypothesized that modifying LNPs to have mixed surfaces (e.g., utilizing two different conjugated lipids derived from different parent lipids) could tune the circulation profile and the transfectability of LNPs.

Accordingly, LNPs having two different conjugated Lipid-PEGs were generated. Each Lipid-PEG component was identified for a specific surface behavior. DSG-PEG2000 (having a saturated lipid tail) was used to impart longer circulation to the LNPs. DOPE(C18:1)-PEGs and Ceramide-PEGs (having lipid tails with unsaturations) were selected as second Lipid-PEGs on the surface to improve the transfectability of the LNPs. The percent composition of the two Lipid-PEGs with saturated and unsaturated lipid tails in the lipid composition (e.g., on the LNP surfaces) were varied in experiments with the aim of generating LNPs with long circulation and high transfectability.

The ability of engineered LNPs FRM146 and FRM237 to deliver DNA to tumors was evaluated in a bilateral H1299 and H1373 dual-xenograft model.

NOD.Cg-Prkdc scid Il2rg tm1Wjl/SzJ mice (NSG, stock 005557, Jackson Laboratory) were used at 7-10 weeks of age at inoculation. mice were anesthetized under isoflurane (2-4%) and placed in the left lateral decubitus position. 1.5×106 H1299 cells or H1373 cells resuspended in 20 uL of PBS:Matrigel (1:2) were percutaneously injected into separate sites (e.g., flanks) in the mice. Control tumor-free mice received 20 uL of PBS:Matrigel. Tumor volume was monitored and randomized by CT imaging.

Approximately 4-5 weeks after tumor inoculation, tumor-bearing and control mice were dosed intravenously (i.v.) with CAG-FLUC DNA-LNP at 1.4 mg/kg. After 48h, whole-body and ex vivo BLI imaging were performed to measure luciferase gene expression as described in Example 3.

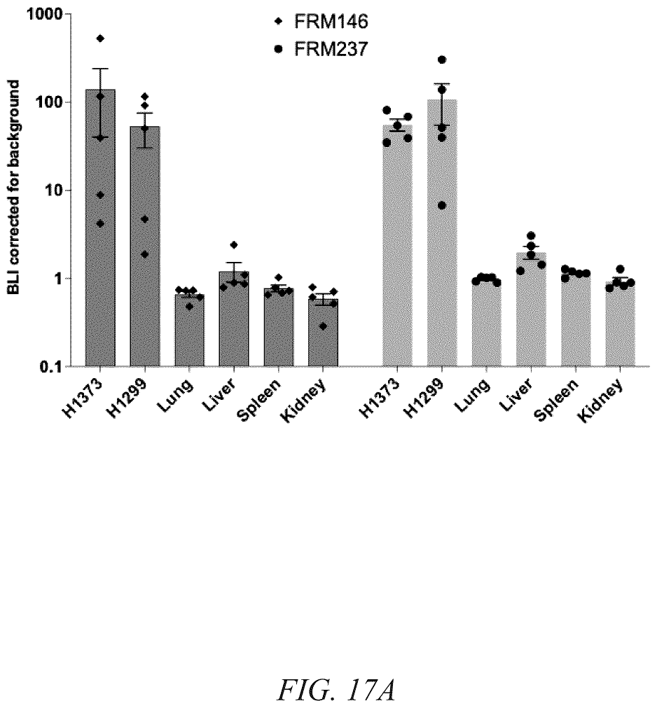

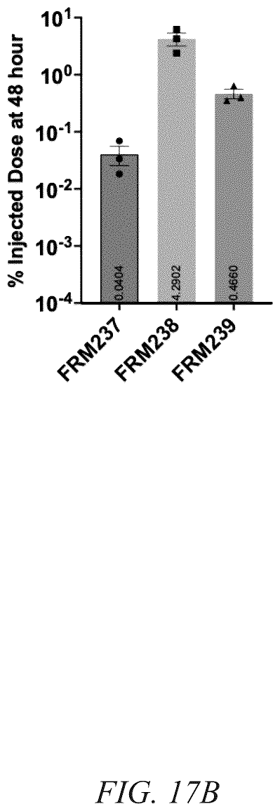

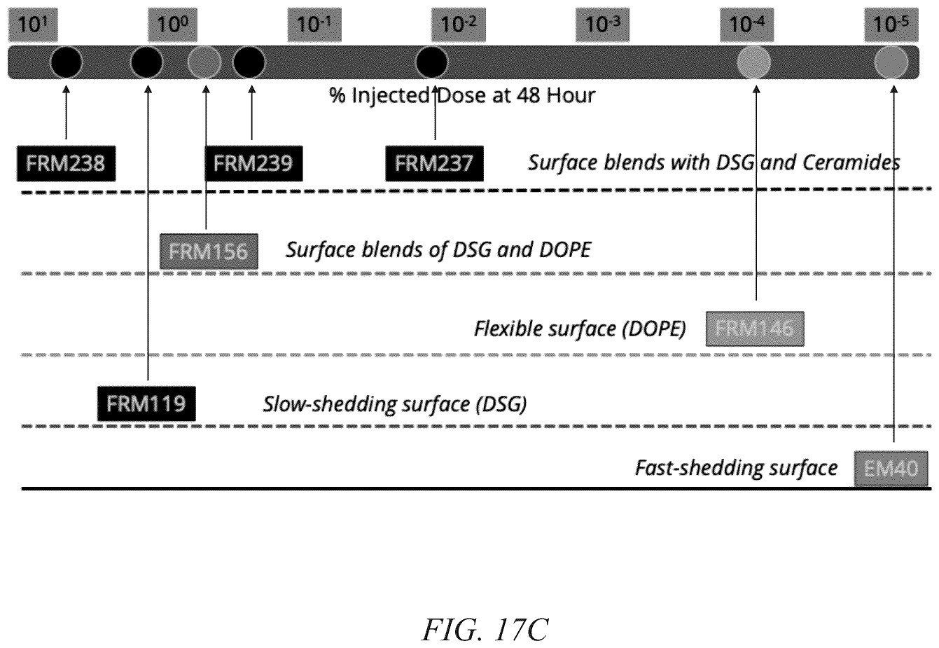

FIG. 17A and FIG. 17B show the result of this experiment. FIG. 17A demonstrates that LNP formulations described herein (“FRM 146”, “FRM237”) having mixed surfaces show selective tumor vs liver targeting despite other LNP modifications. Shown is a graph of BLI intensity of dissected organs 48 hours after injection of the named LNPs into a mouse H1299 and H1373 xenograft model. FIG. 17B and FIG. 17C demonstrate that LNP formulations described herein (“FRM 146”, “FRM237”) having mixed surfaces show long persistence in dosed mice. FIG. 17B shows a graph of the % remaining injected dose of FRM237, FRM238, or FRM239 in the plasma at 48 hours after injection in mice. FIG. 17C shows a summary schematic depicting the different LNP features in the formulations depicted (FRM 238, FRM119, FRM156, FRM239, FRM237, FRM146, and EM40/FRM055) versus the % remaining injected dose at 48 hours.

### Example 11. Evaluation and Engineering of Sterol Compositions to Balance Transfectability and Circulation Profile

### Overview of LNP Engineering Using Cholesterol Derivatives

It was hypothesized that addition of cholesterol carbon-24 alkyl-substituted cholesterol derivatives (e.g., beta-sitosterol and stigmastanol), and particularly carbon-5 saturated cholesterol derivatives (e.g., stigmastanol), would show improved delivery of DNA in cells and in mice when included in an LNP formulation with a high loading of surface lipid-polymer (3 mole %). Accordingly, a series of formulations were prepared wherein cholesterol—the standard sterol component in LNPs—was replaced partially or wholly with an “alternative sterol” (e.g., beta-sitosterol and stigmastanol). The series of LNPs were designed to answer two key questions:
Can the addition of an alternative sterol to a highly shielded particle (e.g., a particle with 3 mol % of surface polymer) lead to higher expression?
Can the ratio of the cholesterol and “alternative sterol” (e.g., beta-sitosterol and stigmastanol) be modified to alter the circulation profile of the particle?
   Background and Rationale for the Incorporation of Alternative Sterols into DNA-LNPs

Cholesterol is a component of many LNP formulations—typically comprising about 40-50 mole % of the formulation. Cholesterol packs into regions of lipid bilayers where other structural lipids (e.g., phospholipids) cannot pack tightly together (“gaps”) to impart additional stability. While LNPs formulated with cholesterol can show adequate stability properties for many applications, it was understood that particles that are too stable may have poor delivery efficiency and that overly high concentrations of cholesterol may be related to poor delivery efficiency. Specifically, it was hypothesized cholesterol might hinder the ability of the ionizable lipid to engage with the endosomal membrane—a necessary step for efficient endosomal escape. Accordingly, it was hypothesized that incorporation of derivatives of cholesterol including those with carbon-24 alkyl-substituents (e.g., beta-sitosterol and stigmastanol) and carbon-5 saturated rings (e.g., stigmastanol) could lead to less tightly packed LNPs which can undergo more efficient endosomal escape to enable higher DNA expression. Moreover, it was hypothesized that the incorporation of these cholesterol derivatives might be critical to achieve higher transfection for LNPs incorporating high surface polymer loadings (e.g., 3 mole % and higher), as we had already identified that such loadings can hinder transfection in vitro (FIG. 11B).

Results from Testing In Vivo

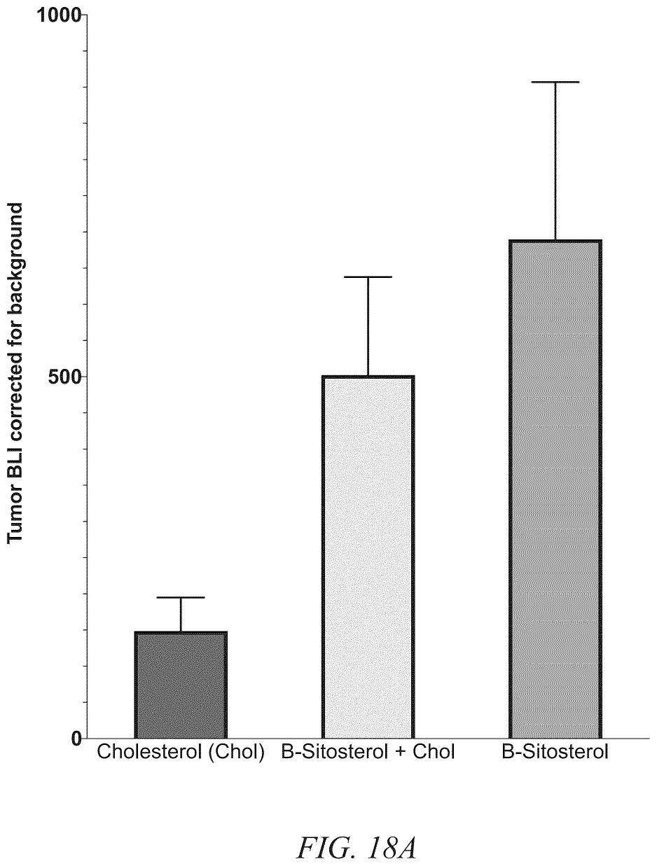

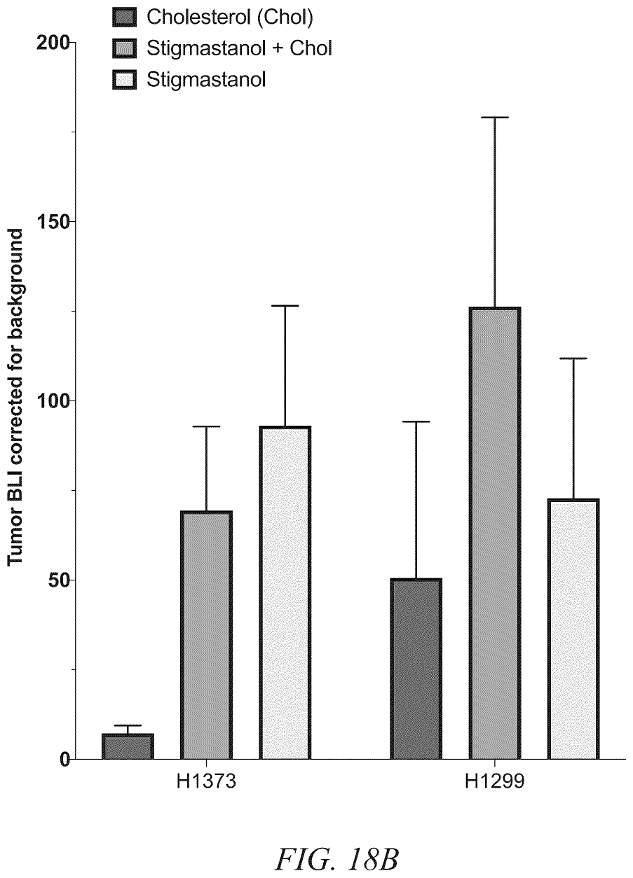

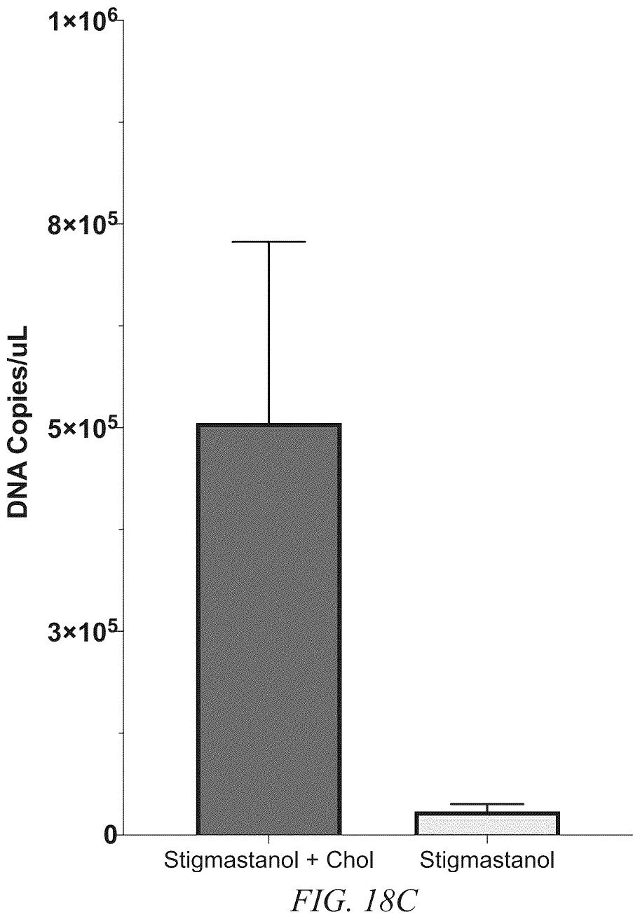

To investigate the effect of partial or full cholesterol replacement with cholesterol derivatives, LNPs encapsulating DNA (e.g., CAG-FLUC DNA) were prepared with MC3, SOPC, a mixture of surface polymers (2% DSG-PEG2k and 1% DOPE-PEG1k) and either cholesterol, beta-sitosterol, or a 50:50 mixture of cholesterol and beta-sitosterol. Each formulation was intravenously administered to a murine H1299 xenograft model prepared as in Example 8 and the tissues were harvested and analyzed for reporter protein expression 48 h post-dose. It was observed that the replacement of cholesterol either partially or fully with beta-sitosterol (B-sitosterol) led to significantly higher expression in the tumor. The results of this experiment are shown in FIG. 18A, which shows a graph of tumor BLI corrected for background from tumors of H1299 xenograft mice transfected with LNPs bearing either cholesterol (“Chol”, FRM441), a 50:50 mix of beta-sitosterol and cholesterol (“B-Sitosterol+Chol” FRM445), or beta-sitosterol alone (“B-Sitosterol” FRM444) as a structural lipid in an LNP formulation. As the conditions with beta-sitosterol resulted in higher expression of the DNA-encoded BLI marker, it was observed that that partial or full replacement of cholesterol with beta-sitosterol resulted in increased DNA expression from the LNP payload in tumors.

To further understand whether the observed improvement in performance was unique to the use of beta-sitosterol, the incorporation of another sterol derivative hypothesized to disrupt tight lipid packing, stigmastanol, was explored. Specifically, formulations comprised of MC3, DSPC or SOPC, the same surface engineering (2% DSG-PEG2k and 1% DOPE-PEG1k), and either cholesterol, stigmastanol, or a 50:50 mixture of cholesterol and stigmastanol were compared. Each formulation was intravenously administered to both a murine H1373 and H1299 xenograft model and the tissues were harvested and analyzed for reporter protein expression 48 h post-dose.

The results are shown in FIG. 18B, which shows a graph of tumor BLI corrected for background from tumors of H1299/H1373 xenograft mice transfected with LNPs bearing either cholesterol (“Chol”), a 50:50 mix of stigmastanol and cholesterol (“Stigmastanol+Chol”, representing FRM225), or Stigmastanol (“Stigmastanol”, representing FRM214) as a structural lipid in an LNP formulation. As the conditions with stigmastanol resulted in higher expression of the DNA-encoded BLI marker, it was observed that that partial or full replacement of cholesterol with stigmastanol resulted in increased DNA expression from the LNP payload in tumors.

TABLE 1F
LNP compositions used in Example 11.

| | FRM441 | FRM445 | FRM444 |
|---|---|---|---|
| DLin-MC3-DMA, mole % | 35 | 35 | 50 |
| SOPC, mole % | 16 | 10 | 10 |
| Cholesterol, mole % | 46 | 26 | 0 |
| B-sitosterol, mo1% | 0 | 26 | 37 |
| DOPE-PEG1000, mole % | 1 | 1 | 1 |
| DSG-PEG2000, mole % | 2 | 2 | 2 |
| N/P ratio | 5.79 | 5.79 | 5.79 |
| Particle size (nm) | 72 | 80 | 85 |
| Zeta potential (mV) | −4.4 | −4.3 | −2.5 |

### Ability to Modify Circulation Profile Using Sterols

Since the use of sterol derivates leads to differences in the packing of lipids within the LNP, it was hypothesized that the use of these components would also impact the rate of surface polymer shedding. Specifically, it was proposed that the replacement of cholesterol with sterols such as beta-sitosterol and stigmastanol would lead to faster PEG shedding, higher cell uptake, and thus a shorter circulation half live in vivo.

To evaluate this concept, pharmacokinetic (PK) analysis was performed for vector copy numbers, e.g., nanoplasmid DNA copies in the plasma to understand the effects on blood circulation. For this preliminary analysis, a single timepoint at 48 h post administration of LNPs was analyzed. The quantity of CAG-FLUC nanoplasmid DNA in plasma was measured using qPCR assays specific to the nanoplasmid DNA sequence. The results are shown in FIG. 18C, which shows a graph of DNA plasma quantity (in units of DNA copies per microliter) in mice injected with at 50:50 mix of sitmastanol and cholesterol (“Stigmastanol+Chol” referring to FRM225) or Stigmastanol alone (“Stigmastanol” referring to FRM214). Based on this data, it was concluded that LNPs where the sterol component is solely a cholesterol derivative like stigmastanol exhibit shorter circulation times than formulations that have a 50:50 mixture of stigmastanol and cholesterol as indicated by the DNA copies in the plasma at 48h.

### Example 12. Evaluation of Decreased Cationic Lipid Concentrations as a Method to Reduce Toxicity or Increase Tolerability

It was hypothesized that cationic lipid concentrations of less than about 50 mol % of the total lipids in the particle would reduce toxicity or increase tolerability of LNP compositions described herein.

Accordingly, LNP formulations were prepared with MC3/Cholesterol/SOPC and different styles of surface polymers or mixtures thereof (denoted surface 1, 2, and 3) with molar ratios of either 50/37/10/3—denoted “50%”—or 35/52/10/3—denoted “35%”. A composition having 3 mol % of a slow-shedding surface polymer (DSG-PEG2k) was denoted surface 1, and compositions having a mixture of a slow shedding PEG and flexible PEG were denoted surface 2 (DSG-PEG2k+DOPE-PEG1k) and surface 3 (DSG-PEG2k+C16ceramide-PEG750). (C16 ceramide-PEG750 can also be described as N-palmitoyl-sphingosine-1-{succinyl[methoxy(polyethylene glycol)750]}herein).

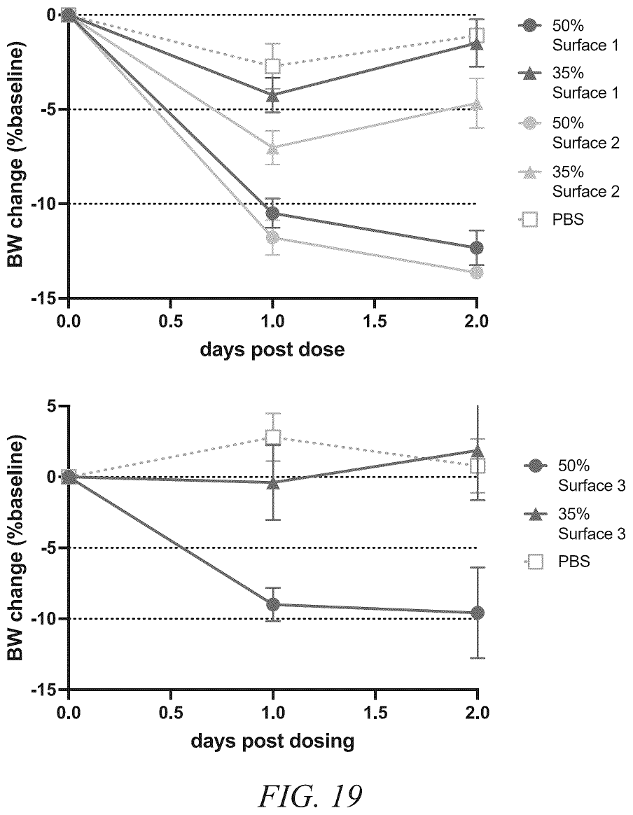

LNP formulations at a fixed dose of DNA per kg of mouse bodyweight were intravenously administered to non-tumor bearing balb/c mice (day 0) and body weight of the mice was tracked over time. FIG. 19 shows the result of this tracking. FIG. 19 depicts a graph showing the percentage change in mouse bodyweight relative to the bodyweight of that mouse before dosing with LNP (y-axis) versus the number of days post-dosing (x-axis). The data indicated that for a given surface, compositions with lower than 50% cationic lipid (e.g., 35%) showed improved tolerability in mice.

TABLE 1G
LNP compositions used in Example 11 alongside labels used in FIG. 19.

| | FRM119 | FRM165 | FRM156 | FRM177 | FRM237 | FRM362 |
|---|---|---|---|---|---|---|
| | (“50% | (“35% | (“50% | (“35% | (“50% | (“35% |
| | surface 1”) | surface 1”) | surface 2”) | surface 2”) | surface 3”) | surface 3”) |
| DLin-MC3- | 50 | 35 | 50 | 35 | 50 | 35 |
| DMA, mole % | | | | | | |
| SOPC, mole | 10 | 10 | 10 | 10 | 10 | 10 |
| % | | | | | | |
| Cholesterol, | 37 | 52 | 37 | 52 | 37 | 52 |
| mole % | | | | | | |
| C16 ceramide | 0 | 0 | 0 | 0 | 1.5 | 1.5 |
| PEG750 | | | | | | |
| DOPE- | 0 | 0 | 1 | 1 | 0 | 0 |
| PEG1000, | | | | | | |
| DSG- | 3 | 3 | 2 | 2 | 1.5 | 1.5 |
| PEG2000, | | | | | | |
| mole % | | | | | | |
| N/P ratio | 5.79 | 5.79 | 5.79 | 5.79 | 5.79 | 5.79 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-60. (canceled)

61. A method, comprising:
administering to a subject a composition comprising a nucleic acid encoding an engineered polypeptide, wherein said engineered polypeptide comprises:
(a) an extracellularly-oriented domain comprising an epitope capable of binding: (i) an antibody; or (ii) a peptide hormone or growth factor; wherein said extracellularly-oriented domain does not comprise an scFv; and
(b) a transmembrane domain or membrane affinity domain capable of associating with an outer membrane of a cell, comprising a transmembrane domain or membrane affinity domain from DLL3, PSMA, SSTR2, PD-L1, EGFR, CD28, CD4, ICOS, or CD73,
wherein (a) and (b) are heterologous to each other.

62. The method of claim 61, wherein said engineered polypeptide does not comprise a light chain variable (VL) domain.

63. The method of claim 61, wherein said engineered polypeptide does not comprise an intracellular portion of a CD3zeta, CD137, or CD28 polypeptide.

64. The method of claim 61, wherein said epitope capable of binding: (i) said antibody or (ii) said peptide hormone or growth factor is derived from DLL3, PSMA, SSTR2, or any combination thereof.

65. The method of claim 64, wherein said epitope is derived from DLL3.

66. The method of claim 65, wherein said epitope comprises about 15 to about 260 contiguous residues of an extracellular domain of DLL3 having at least 80% identity to SEQ ID NO: 5, or a variant thereof.

67. The method of claim 64, wherein said epitope comprises a sequence having at least 80% sequence identity to any one of SEQ ID NOs: 4, 5, 19, 20, 64, 65, or a variant thereof.

68. The method of claim 61, wherein said transmembrane domain or said membrane affinity domain capable of associated with said outer membrane of said cell comprises a sequence having at least 80% sequence identity to a transmembrane domain of any one of SEQ ID NOs: 68-76.

69. The method of claim 61, wherein said nucleic acid further comprises: (i) an open reading frame (ORF) encoding said engineered polypeptide; (ii) and a promoter operably linked to said ORF.

70. The method of claim 69, wherein said promoter is: (i) not a T-cell specific promoter; or (ii) not a TCRA, TCRB, CMV, EF-1, hPGK, CD3, or RPBSA promoter.

71. The method of claim 69, wherein said promoter is a promoter of a gene overexpressed in a cancer cell versus a normal cell, or a functional fragment thereof.

72. The method of claim 61, wherein said nucleic acid further comprises a vector encoding said engineered polypeptide, wherein said vector comprises a nanoplasmid vector, a close-ended linear duplex (CELiD) vector, or a Doggybone DNA vector (dbDNA).

73. The method of claim 72, wherein said vector comprises a nanoplasmid vector.

74. The method of claim 73, wherein a nanoplasmid vector comprises a minimized bacterial ColE1 or R6K origin of replication and a bacterial RNA-based selectable marker.

75. The method of claim 61, further comprising administering to said subject an antibody or antigen-binding fragment thereof, a protein ligand or functional fragment thereof, or a small molecule configured to bind said epitope.

76. The method of claim 75, wherein said antibody or antigen-binding fragment thereof, said protein ligand or said functional fragment thereof, or said small molecule configured to bind said epitope is administered intravenously.

77. The method of claim 75, wherein: (i) said administering to said subject said composition comprising said nucleic acid; and (ii) said administering to said subject said antibody or antigen-binding fragment, said protein ligand or functional fragment thereof, or said small molecule configured to bind said epitope are separated by 96 hours or less.

78. The method of claim 75, further comprising detecting binding of said antibody or antigen binding fragment, said protein ligand or functional fragment thereof, or said small molecule to said epitope.

79. The method of claim 78, further comprising detecting binding of said antibody or antigen binding fragment, said protein ligand or functional fragment thereof, or said small molecule to said epitope via a non-invasive imaging method.

80. The method of claim 79, wherein said non-invasive imaging method is performed 120 hours or less after administering to said subject said antibody or antigen-binding fragment, said protein ligand or functional fragment thereof, or said small molecule configured to bind said epitope.

81. The method of claim 75, wherein said antibody or antigen binding fragment thereof or said protein ligand or functional fragment thereof further comprises a radioisotope.

82. The method of claim 81, wherein said radioisotope is a positron-emitting radioisotope.

83. The method of claim 79, wherein said non-invasive imaging method comprises positron emission tomography (PET).

84. The method of claim 75, wherein said antibody or antigen binding fragment thereof or said protein ligand or functional fragment thereof further comprises a magnetic resonance imaging (MRI) contrast agent.

85. The method of claim 84, wherein said non-invasive imaging method comprises MRI.

86. The method of claim 75, wherein said antibody or antigen binding fragment thereof or said protein ligand or functional fragment thereof comprises an antibody or antigen binding fragment thereof.

87. The method of claim 86, wherein said antibody or antigen binding fragment thereof comprises an antibody drug conjugate (ADC).

88. The method of claim 61, wherein said engineered polypeptide comprises an extracellularly-oriented polypeptide spacer domain of about 15 to about 40 angstroms in length when folded between (a) and (b)

89. The method of claim 88, wherein said extracellularly-oriented polypeptide spacer domain comprises a hinge sequence from IgG4, IgG1, IgG2, IgG3, IgK, CD4, or CD28, or any combination thereof.

90. The method of claim 88, wherein said extracellularly-oriented polypeptide spacer domain comprises a hinge sequence having at least 80% sequence identity to a hinge sequence of any one of SEQ ID NOs: 58, 59, 60, 61, 62, 66, 67, or a variant thereof.


SEQUENCE LISTING (2 sequences)

SEQ ID NO: 1 - DNA, 281 bp, Escherichia coli
  source 1..281: mol_type = genomic DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 2 - DNA, 139 bp, Escherichia coli
  source 1..139: mol_type = genomic DNA
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta